(19) United States
(12) Patent Application Publication
(10) Pub. No.: US 2023/0295230 A1
(43) Pub. Date: Sep. 21, 2023

(54) TRANSMEMBRANE BETA BARREL PROTEINS

(71) Applicants: University of Washington, Seattle, WA (US); THE UNIVERSITY OF LEEDS, a body incorporated by Royal Charter (RC: 000658), West Yokshire (GB)

(72) Inventors: Anastassia VOROBIEVA, Seattle, WA (US); David BAKER, Seattle, WA (US); James Ea HORNE, West Yokshire (GB)

(21) Appl. No.: 18/041,045

(22) PCT Filed: Sep. 2, 2021
(86) PCT No.: PCT/US2021/048802

§ 371 Date: Feb. 8, 2023

Related U.S. Application Data
(60) Provisional application No. 63/074,722, filed on Sep. 4, 2020.

(51) Int. Cl. C07K 7/06; C12N 15/63

(52) U.S. Cl. CPC C07K 7/06; C12N 15/63

(57) ABSTRACT

The present disclosure provides non-naturally occurring beta barrel proteins as defined, self-complementing multipartite beta barrel proteins, uses of such proteins, and methods for designing such proteins.

### CROSS REFERENCE

This application claims priority to U.S. Provisional Pat. Application Serial No. 63/074722 filed Sep. 4, 2020, incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Aug. 31, 2021 having the file name “20-1273-WO-SeqList_ST25.txt” and is 32kb in size.

### BACKGROUND

The de novo design of an integral transmembrane β-barrel (TMB) has not yet been achieved. TMBs can spontaneously fold into lipid bilayers from an unfolded chain, possibly through a mechanism involving concerted membrane insertion and folding of the β-hairpins. How this folding in a non-aqueous environment is encoded in the sequences of TMBs is not well understood because of experimental challenges in characterizing the rugged folding pathway - including possible off-pathway, misfolded or “invisible” states, and the often nonsuperimposable folding and unfolding equilibria (hysteresis).

### SUMMARY

In one aspect, the disclosure provides non-naturally occurring beta barrel proteins comprising the formula X1-Z1-X2-Z2-X3-Z3-X4-Z4-X5-Z5-X6-Z6-X7-Z7-X8-Z8, wherein:
X1 comprises at least two amino acid residues, wherein the C-terminal residue in X 1 is G;
Z1 is a beta strand consisting of 10 amino acid residues, wherein residue 1 is S, T or D, residue 9 is G and residue 10 is W or Y, and wherein residues 2, 4, 6, and 8 are hydrophobic residues or G;
X2 is a loop comprising at least 5 amino acids;
Z2 is a beta strand consisting of 12 amino acid residues, wherein residues 5 and 6 are G, residue 9 is Y, residue 12 is S, T, or D or wherein residue 12 is S or T, and residues 1, 3, 7, and 11 are hydrophobic residues or G;
X3 is a beta turn consisting of two amino acids in length;
Z3 is a beta strand consisting of 9 amino acid residues, wherein residues 6 and 8 are G, residues 7 and 9 are W or Y, and residues 1, 3 and 5 are hydrophobic residues or G;
X4 is a loop comprising at least 5 amino acids;
Z4 is a beta strand consisting of 14 amino acid residues, wherein residue 1 is N or Q, residues 6-8 are G, residue 11 is Y, residue 14 is S, T, or D or wherein residue 14 is S or T, and residues 3, 5, 9, and 13 are hydrophobic residues or G;
X5 is a beta turn consisting of two amino acids in length;
Z5 is a beta strand consisting of 11 amino acid residues, wherein residue 3 is P, residue 8 is G, residue 11 is Y or W, and residues 1, 5, 7, and 9 are hydrophobic residues or G;
X6 is a loop comprising at least 5 amino acids;
Z6 is a beta strand consisting of 14 amino acid residues, wherein residue 3 is P, residues 6 and 8 are G, residue 11 is Y, residue 14 is S, T, or D or wherein residue 14 is S or T, and residues 1, 5, 7, 9, and 13 are hydrophobic residues or G;
X7 is a beta turn consisting of two amino acids in length;
Z7 is a beta strand consisting of 9 amino acid residues, wherein residue 8 is G, residues 7 and 9 is W or Y, and residues 1, 3, and 5 are hydrophobic residues or G;
X8 is a loop comprising at least 5 amino acids;
Z8 is a beta strand consisting of 12 amino acid residues, wherein residue 1 is N or Q, residue 6 is G, residue 9 is Y, and residues 1, 3, 5, 7, and 11 are hydrophobic residues or G.

In various embodiments that may be combined, the C-terminal residues in X1 are PG or QG; residue 1 in Z1 is S or T; none of X2, X4, X6, or X8 comprise consecutively the amino acid residues across a single row of Table 1; X3, X5, and X7 independently have P, E, or D at residue 1; and N, G, E, D, Q. or Y at position 2; Z1 residue 5 is Y, Z5 residue 4 is Y, or both; X2, X4, X6, or X8 each independently comprise an amino acid sequence selected from the group consisting of the amino acid sequence of SEQ ID NOS:22-26; and/or residue 2 of X2 is Y.

In one embodiment, one or more of the following is true:
Z1 residue 8 is A;
Z3 residue 5 is A;
Z5 residue 7 is A;
Z6 residue 5 and residue 7 are A or G; and/or
Z8 residue 5 is A or G.

In another embodiment, one or both of the following is true:
Z3 residue 4 is E or D and Z1 residue 5 is Y; and/or
Z7 residue 6 is E or D and Z5 residue 4 is Y.

In other embodiments that may be combined, one or more of X1, X2. X4, X6, and X8 comprise an added functional domain; the polypeptide comprises an added functional domain C-terminal to Z8; and the protein comprises the amino acid sequence at least 50%, 55%, 60%, 65%, 70%, 75%, 80%, 85%, 90%, 95%, or 1 00% identical to the amino acid sequence selected from SEQ ID NOS: 1-21, wherein residues in parentheses are optional and may be present or absent.

In another aspect the disclosure provides non-naturally occurring, self-complementing multipartite beta barrel protein, comprising at least a first polypeptide component and a second polypeptide component, wherein the at least first polypeptide component and the second polypeptide component are not covalently linked, wherein in total the at least first polypeptide component and the second polypeptide component comprise domains X1-Z1-X2-Z2-X3-Z3-X4-Z4-X5-Z5-X6-Z6-X7-Z7-X8-Z8, wherein each domain is as defined herein;

wherein (a) each beta strand is fully present within one polypeptide component of the at least first polypeptide component and the second polypeptide component, (b) none of the at least first polypeptide component and the second polypeptide component include each of Z1, Z2, Z3, Z4, Z5, Z6, Z7, and Z8; and (c) one of domains X2, X4, X6, and X8 may be partially or wholly absent in each of the first polypeptide and the second polypeptide.

In other aspects, the disclosure provides nucleic acids encoding the beta barrel protein or the first or second polypeptide of any embodiment, expression vectors comprising the nucleic acid operatively linked to a control sequence, recombinant host cell comprising the proteins, polypeptide components, nucleic acids and/or the expression vector of the disclosure, pharmaceutical compositions, and methods for use and design of the proteins, split proteins, and polypeptide components of the disclosure.

### DESCRIPTION OF THE FIGURES

FIG. 1. Principles for designing TMBs backbones. In panels A-D, the membrane anchoring residues are shown as spheres hatched. (A, B) Geometric model of membrane-association constraints on the β-barrel architecture. (A) Asymmetric register shifts between the β-hairpins can be accommodated by tilting the β-barrel to the transmembrane axis by an angle α= arctan(Z/C). (B) To place asymmetric register shifts on the trans and cis membrane boundaries, the distances between the cis anchor residue N and all anchor residues in trans were calculated and projected to the horizontal plane. θ is the angle of the β-strands to the main β-barrel axis. (C) The geometric model (center) and Rosetta® modelling followed by hydrophobic thickness prediction with PPM (right) predict similar tilt angles (α) of the β-barrel to the membrane axis for a given β-strand arrangement: both models show inconsistent hydrophobic thickness for β-barrel architectures with double register shifts are located on strand 1 in cis (strand N) (Top) and on strand 6 in trans (strand N+5) (Bottom). (D and E) 2D schematic representation of the connectivity (hydrogen bonds as dashed lines) between β-strands in the TMB designs. Side-chains are shown as spheres and glycine residues as black dots. (D) Aromatic girdle motifs on the surface of the β-barrel are shown as side chains. Prolines are shown as pentagons. (F) The tyrosines of the mortise/tenon motifs are shown as side-chains. Glycine kinks were arranged to bend the β-sheet into four corners (vertical arrows). (F) Comparison of the backbone hydrogen bond geometries in water soluble (top) and transmembrane (bottom) β-barrels with 8 β-strands; data for glycine kink residues (center, residue II.), residues preceding a glycine kink (right, residue III.) and other residues (left, residue I.) are plotted separately. An example of glycine kink (residue II.) with an aromatic rescue interaction is shown in the central panel, with water molecules shown as dots.

FIG. 2. Negative design is critical for de novo TMB folding. (A) Successful design of TMBs requires reducing β-sheet propensity. X axis: β-sheet propensity (calculated with RaptroX® (62), y axis: hydrophobicity of the core (GRAVY hydropathy index (63)). Labels indicate folded species was validated by HSQC; Greenk, naturally occurring TMBs with 8 strands. Circle size, aggregation propensity of the sequence predicted with TANGO (64). (B) Experimental workflow. The number of unique designs (excluding loop doublons) satisfying each criteria is shown in brackets. (C and D) Proper folding of tOmpA requires negative design against strong β-turn nucleating sequences on the trans side. Left: Rosetta® energy landscapes of designs with canonical low energy (C) or sub-optimal (D) sequences substituted in a 3:5 type I β-turn with a GI β-bulge. Conformational perturbations were generated using Kinematic Loop Closure (65); the inset shows the backbone conformations of the twenty-five lowest-energy models. Center: After refolding in 2X CMC DDM detergent, OmpTrans3 elutes on SEC similarly to tOmpA (arrow, 14.62 ml for OmpTrans3 and 14.53 ml for tOmpA) and runs as a heat modifiable species on SDS-PAGE characteristic of folded tOmpA, while the OmpAAG peak elutes earlier ( 13.96 ml) and does not show a band shift. Right: The far-UV CD spectrum of OmpTrans3, but not OmpAAG, is similar to that of tOmpA.

FIG. 3. Biophysical characterisation of de novo designed TMB2.3 and TMB2.17 vs tOmpA in synthetic lipid membranes. (A) Urea dependence of folding and unfolding in DUPC LUVs. The fluorescence intensity at 335 nm was plotted against urea concentration to determine the midpoint urea concentration for folding (Cₘ^{F}) (open circles, dashed line) and unfolding (C-ₘ^{UF}) (filled circles, solid line). Kinetics of folding into (B) DUPC and (C) DMPC LUVs at an LPR of 3200:1 (mol/mol) in 50 mM glycine-NaOH pH 9.5, 2 M urea at 25° C. monitored by tryptophan fluorescence at 335 nm over 30 minutes (line). Data were fitted with a single exponential function to determine folding rate constants (dashed line). Three replicates are shown for each.

FIG. 4. Crystal structure of 7. (A-F) Superposition to the design model and comparison to the crystal structure of the naturally occurring tOmpA (PDB ID: IQJP). (A) Full backbone superposition. (B) Comparison of the transverse β-barrel cross-section geometries. (C) Superposition of the β-strands around a mortise-tenon motif, showing the extended backbone conformation of the glycine kink (G27) and the rotamer of the tyrosine involved in the aromatic rescue interaction (Y11) which are nearly identical in crystal structure and design model. (D) Superposition of the side-chains involved in the core network of polar interactions around the two mortise-tenon motifs. The black lines indicate the locations of the four transverse slices for which core packing is shown in for the design model and crystal structure (H; the two are very similar) and compared to core packing in tOmpA (1) which is quite different Cα atoms are shown as spheres; the positions of the tyrosines in the mortise/tenon folding motifs are labeled.

FIG. 5. Structural constraints on the β-barrel architecture. (A) Comparison between the overall architecture of the previously reported de novo designed water-soluble β-barrels (mFAPs) and the native tOmpA. Both the water-soluble and membrane protein can be oriented in the same way based on the chirality of the β-strand connections and the location of the N- and C-termini, with the “bottom” of the mF APs corresponding to the cis side of tOmpA; and the “top” to the transmembrane “trans” side. (B) The β-barrel architecture is defined by the number of β-strands (N) and the shear number S. S is the number of register shifts along a given β-strand after circling the whole B-barrel in the direction of the hydrogen bonds. S equals the number of Cβ strips in the barrel; half of which point to the β-barrel lumen. (C, D) The combination of the shear number and of the number of strands define the packing arrangement of side-chains in the core of the β-barrel. For a given number of βstrands (N=8), the core of the β-barrel of type (S=N) is packed with a 4-fold symmetric arrangement of side-chains (C, side-chains are represented as spheres and colored according to their position in their respective Cβ-strip). (D) The packing symmetry is broken when the shear number is increased by two register shifts so that (S=N+2). The asymmetric arrangement increases the degree of contact between the intertwined side-chains.

FIG. 6. Constraints on the structure and sequence of the cis β-turns. (A-D) Representative structures of common canonical type I (A), type I′ (B), type II′ (C) and type I with GI β-bulge (D) and their membrane context in our model (supported by predictions with the PPM server). The membrane anchoring residue (i) is highlighted with a sphere. Hydrogen bond interactions are shown as black dashes. (E, F) β-barrel architecture (E) and cross-section (F) comparisons between the TMB de novo designs, the previously reported soluble de novo designed mini Fluorescence Activating Protein 1 (mFAP1) and the transmembrane domain of the native Outer Membrane Protein A from E. coli (tOmpA). (G) Comparison of the cis β-turn sequences in tOmpA (SEQ ID NO: 28), in mFAPs (SEQ ID NO: 27) and concensus (SEQ ID NO: 29) used for TMB design. The β-turn residues are shown in bold, the β-bulge residue is underlined, the tyrosine of the aromatic girdle is red and hydrophobic residues are shown in grey. (H-K) Heatmaps showing the amino acid preference per position for cis β-turns with canonical backbone conformations in natural transmembrane and water-soluble β-barrels.

FIG. 7. Mathematical formula to calculate the vertical and horizontal offset between two residues in the β-barrel as a function of the angle θ of the β-strands to the main barrel axis. (A) The vertical offset between two anchor residues on the same side of the β-barrel was obtained by calculating the difference between the vertical offset when moving from strand to strand along the hydrogen bonds (A) and the vertical offset when moving along one β-strand (B). (B) The horizontal offset between two anchor residues located on the opposite sides of the β-barrel was obtained by calculating the difference between the horizontal offset when moving from strand to strand along the hydrogen bonds (A′) and the horizontal offset when moving along one β-strand (B′). The number of residues z is a function of the desired hydrophobic thickness (see examples). The tilt angle θ of the βstrands to the main axis of the β-barrel is a function of the parameters n and S (see examples).

FIG. 8. Membrane-association constraints on the β-barrel architecture (part 2). (A-D) Relationship between the topology (left), the geometric model (center) and the Rosetta® molecular model coupled with PPM lipid bilayer prediction (right) of four βbarrels with 8 strands and a shear number of 10 and different register shift distributions. From the N- to the C-tenninus: (A) register shifts 0+6+2+2 (topology N=4;6); (B) register shifts 4+2+2+2 (topology N=2;4); (C) register shifts 2+2+4+2 (topology N=6;4); (D) register shifts 2+2+2+4 (topology N=8;4). (E-G) Average hydrophobic thickness (E), energy of transfer from water to lipid (F), and tilt angle to the membrane axis (G) predicted by the PPM server on 20-25 Rosetta® models. The more uneven distribution of register shifts (A) results in a more tilted β-barrel. The topologies (B), (C) and (D) differ only by the positions of the four-residue register shift in the β-sheet These three topologies and the one presented in the main text (register shifts 2+4+2+2; N=4;4) result in very similar predicted interaction with the lipid bilayer and differ only in the direction of the tilting to the membrane axis.

FIG. 9. The resurface water-soluble β-barrel designs have high aggregation propensity. The aggregation propensity of sequences obtained by redesigning the surface of water-soluble β-barrels with hydrophobic residues (surface re-purposing) or designed completely from scratch (de novo design) was predicted using PASTA®2.0 (94), TANGO® (64) and AGGRESCAN® (95) prediction servers. All three servers predicted higher aggregation propensity for the “surface re-purposed” designs.

FIG. 10. Positions of mortise/tenon motifs in some naturally occurring TMBs. (A-C) Two extended-definition mortise/tenon motifs (YGD/E) found in the native tOmpA TMB mapped on tOmpA topology (A) and structure (B). (C) Weblogo (96) representation of the amino acid diversity in the MSA of tOmpA homologs for residues of the YGD/E motifs (black box) and residues from the second shell of polar interactions. (D) Putative mortise/tenon motifs identified in two native TMBs with β-barrel architecture (n=10,S=12) and mapped on the 2D representation of the β-barrel topology. (E) Putative mortise/tenon motifs identified in four native TMBs with β-barrel architecture (n=10,S=12) and mapped on the 2D representation of the β-barrel topology. (F) Legend of the pictograms used in panels (A), (D) and (E).

FIG. 11. Frequency of amino acids in de novo MB designs and natural TMBs. (A) The amino acid frequencies in native 8-strands TMBs derived from the MSAs were validated against previously published frequencies obtained from crystal structures of natural TMBs of different numbers of strands (8). (B and C) Amino acid distributions in the core and on the surface of the reference TMB set.. (D) Frequency of amino acids in sequences generated in the sets of designs TMB0, TMB1 and TMB2. The distributions are broken down into core and surface positions and compared to the reference set obtained from the MSA in (A). (E and F) Frequency of each amino acid on the aromatic girdle position on the cis hairpins (E, three positions away from the cis β-turn on strand 1) and on the trans hairpins (F, four positions away from the trans β-turn on strand 1).

FIG. 12. Naturally occurring β-turns on the trans side of TMBs have sub-optimal sequences for the backbone conformations observed in crystal structures (part 1). (A) Backbone conformation characteristic of the 3:5 type I (β-turn with a GI bulge. The hydrogen bonds are shown as black dashed lines. The residues are numbered from residue i (last residue of the first β-strand to i+4 (first residue on the second β-strand). Part of the neighbour β-strand is shown on the right. (B) Heatmap showing per position amino acid preference in 3:5 type 1 β-turns fragments extracted from the PDB (and biased toward watersoluble protein statistics). The sequence SDG results in a tight intra-turn hydrogen bond network (A). (C) Rosetta“-” p_aa_pp scores computed on 100 trans and 119 cis β-turn residues (two to five-residue β-turns) extracted from 13 crystal structures. (D) Structure to energy landscape computed with Rosetta® loopmodel protocol with KIC (65) for the canonical (SSDGK) and sub-optimal β-turn sequences found in natural TMBs. The x axis shows the RMSD of the simulated conformation to the canonical backbone of the 3:5 type 1 S-turn.

FIG. 13, Expression gels of designs from set0 with long loops in trans. SDS-PAGE gels showing whole cells expressing native (full length OmpA and OmpSDG) and designed (TMB0.1 and TMB0.5 which have inserted native loop sequences (comp) or scrambled loop sequences (.scr) from tOmpA) constructs at to (induction), t1, t2 and t3 (one, two and three hours after induction of protein expression). The red arrow shows the expected molecular weight (Mw) for each construct.

FIG. 14. Experimental characterization of OmpTrans variants of tOmpA. (A) SEC chromatogram of tOmpA refolded into DDM detergent micelles. The band-shift assay on SDS-PAGE shows the presence of two different heat-modifiable species that match the two major peaks of the chromatogram. The existence of oligomeric OmpA species has been described (97). (B-D) SEC chromatogram of OmpTrans1, OmpTrans2 and OmpTrans4 refolded into DDM detergent micelles. (E) Far-UV CD spectra collected for tOmpA in DDM micelles at temperatures ranging from 25° C. to 95° C. (F) Far-UV CD spectra collected for OmpTrans1 in DDM micelles at temperatures ranging from 25° C. to 95° C.

FIG. 15. Biophysical characterization of the OmpTrans3 variant of tOmpA in synthetic lipid membranes. (A) Urea dependence of folding and unfolding in DUPC LUVs. The fluorescence intensity at 335 nm was plotted against urea for folding (open circles, dashed line) and unfolding (filled circles, solid line). OmpTrans3 is able to fold even in 9 M urea. Kinetics of folding into (B) DUPC and (C) DMPC LUVs at an LPR of 3200:1 (mol/mol) in 50 mM glycine-NaOH pH 9.5, 2 M urea at 25° C. monitored by tryptophan fluorescence at 335 nm over 30 minutes (red line). Data were fitted with a single exponential function to determine folding rate constants (black dashed line). Three replicates are shown for each.

FIG. 16. Designed OMPs have β-sheet secondary structure. Far UV CD spectra of (A) TMB2.3 and (B) TMB2.17 refolded overnight at 25° C. in DUPC LUVs in 50 mM glycine-NaOH pH 9.5 containing 0.24 M urea, 2 M urea or 8 M urea. A spectrum was also acquired in 8 M urea without lipid (red dashed).

FIG. 17. SDS-PAGE band-shift folding assays. (A) tOmpA. (B) TMB2.3, (C) TMB2.17 and (D) OmpTrans3 were refolded overnight at 2° C. in DUPC LUVs at a lipid-to-protein ratio (LPR) of 600:1 in 50 mM glycine-NaOH pH 9.5 containing 0.24-8 M urea. Samples were run on 15% (w/v) acrylamide/bis-acrylamide (37.5:1 w/w) Tris-tricine gels to resolve folded and unfolded species. The boiled sample was heated to >95° C. for 10 minutes prior to loading.

FIG. 18. Tryptophan fluorescence emission spectra of folded OMPs. (A) tOmpA, (B) TMB2.3, (C) TMB2.17 and (D) OmpTrans3 folded after 30 minutes at 25° C. in DUPC LUVs at an LPR of 3200: 1 (mol/mol) in 50 mM glycine-NaOH pH 9.5 containing 2 M urea. The spectra show a fluorescence maximum at 335 nm indicative of the folded state. Three replicates are shown for each.

FIG. 19. Designed TMBs are unable to fold in 9 M urea or without lipids. Kinetics of TMB folding were monitored by tryptophan fluorescence emission intensity at 335 nm. (A) OMPs were diluted into DUPC LUVs at an LPR of 3200:1 (mol/mol) in 50 mM glycine-NaOH pH 9.5 in 9 M urea at 25° C., and (B) TMBs were diluted in 50 mM glycine-NaOH pH 9.5 in 2 M urea at 25° C. in the absence of lipid. TMBs show no folding in 9 M urea over the timescales investigated (30 minutes), with the exception of OmpTrans3 which folds with slow kinetics under these conditions. These TMBs do not fold in 2 M urea in the absence of lipids.

FIG. 20. NMR spectrometry results validate the number of strands and the shear number of the design TMB2.3. (A) Coverage of the peak assignments mapped on the sequence of TMB2.3 (SEQ ID NO: 1). (B) Residues showing multiple resonance peaks in the NMR experiment mapped onto the 3D model of the TMB2.3 design. (C) Secondary structures predicted based on secondary chemical shifts using TALOS-N® and mapped on the TMB2.3 sequence. The pictogram in the bottom of the figure and the color show the secondary structure properties in the design model. (D) Secondary structure NMR predictions and NOEs mapped on the sequence of TMB2.3 (SEQ ID NO: 30).

FIG. 21. Per residue chemical shifts and Random Coil Index (RCI S2) derived from the NMR profile of TMB2.3 in DPC detergent micelles. The positions of glycine kink residues are marked with stars. The cis β-turns are highlighted by boxes (including the associated β-bulge residue) and the trans β-turns are highlighted by boxes. (A) Cα chemical shifts of the assigned residues in the β-barrel. (B) Cα-Cβ chemical shifts of the assigned residues in the β-barrel. For glycine residues, the Cβ chemical shifts are set to 0. (C) Random coil index predicted with the TALOS-N® software based on the chemical shifts.

FIG. 22: Comparison of the architecture and sequences of the TMB2.3 and TMB2.17 designs to native tOmpA. (A) Comparison of the topology diagrams generated with PDBsum (98) with the Rosetta® model TMB2.3 and the crystal structure of the native tOmpA. (B) Alignments of TMB2.3 (SEQ ID NO: 1), TMB2.17 (SEQ ID NO: 2) and tOmpA (SEQ ID NO: 31) sequences mapped to the secondary structure to TMB2.3. The cis loops of tOmpA have been truncated to facilitate the graphical representation. Special positions in the sequences are highlighted (legend on the right).

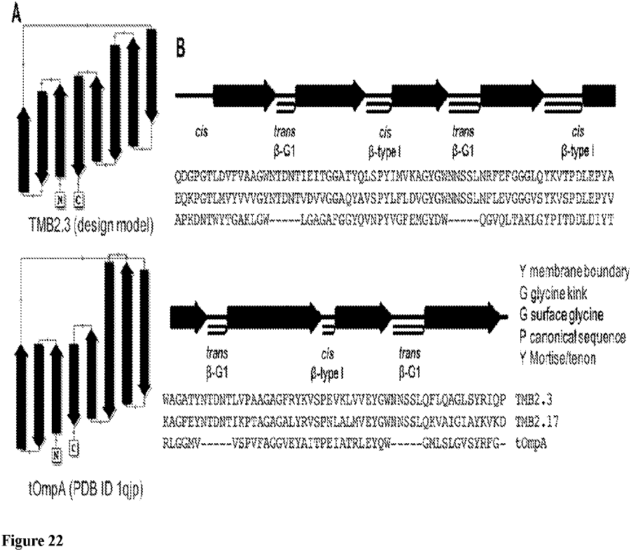

FIG. 23. TMB backbones relaxed with proline at position 67 preceding the Tyr68 of a mortise/tenon motif stabilizes the aromatic rescue conformation. (A) The tyrosine rotamer characteristic of the aromatic rescue interaction is more favorable (lower fa_dun energy) in the presence of Pro67. (B,C) Tyr68 (B) and G88 (C) in the mortise tenon motif have lower energy based on Rosetta® total_score. (D) The presence of Pro67 enables a more extended conformation for Gly66 glycine kink with a negative Ψ angle. (E) The presence of Pro67 enables a more extended conformation for Gly66 glycine kink with more pronounced out-of-plane backbone hydrogen bonds.

### DETAILED DESCRIPTION

All references cited are herein incorporated by reference in their entirety. As used herein, the singular forms “a”, “an” and “the” include plural referents unless the context clearly dictates otherwise.

As used herein, the amino acid residues are abbreviated as follows: alanine (Ala; A), asparagine (Asn: N), aspartic acid (Asp; D), arginine (Arg: R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser: S), threonine (Thr; T), tryptophan (Trp; W), tyrosine (Tyr, Y), and valine (Val; V).

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In one aspect, the disclosure provides non-naturally occurring beta barrel proteins comprising the formula X1-Z1-X2-Z2-X3-Z3-X4-Z4-X5-Z5-X6-Z6-X7-Z7-X8-Z8, wherein:
X I comprises at least two amino acid residues, wherein the C-terminal residue in X 1 is G;
Z1 is a beta strand consisting of 10 amino acid residues, wherein residue 1 is S, T or D, residue 9 is G and residue 10 is W or Y, and wherein residues 2, 4, 6, and 8 are hydrophobic residues or G;
X2 is a loop comprising at least 5 amino acids;
Z2 is a beta strand consisting of 12 amino acid residues, wherein residues 5 and 6 are G. residue 9 is Y, residue 12 is S, T, or D or wherein residue 12 is S or T, and residues 1. 3, 7, and 11 are hydrophobic residues or G;
X3 is a beta turn consisting of two amino acids in length;
Z3 is a beta strand consisting of 9 amino acid residues, wherein residues 6 and 8 are G, residues 7 and 9 are W or Y, and residues 1, 3 and 5 are hydrophobic residues or G;
X4 is a loop comprising at least 5 amino acids;
Z4 is a beta strand consisting of 14 amino acid residues, wherein residue 1 is N or Q, residues 6-8 are G, residue 11 is Y, residue 14 is S. T, or D or wherein residue 14 is S or T, and residues 3, 5, 9, and 13 are hydrophobic residues or G;
X5 is a beta turn consisting of two amino acids in length;
Z5 is a beta strand consisting of 11 amino acid residues, wherein residue 3 is P, residue 8 is G, residue 11 is Y or W, and residues 1, 5, 7, and 9 are hydrophobic residues or G;
X6 is a loop comprising at least 5 amino acids;
Z6 is a beta strand consisting of 14 amino acid residues, wherein residue 3 is P, residues 6 and 8 are G, residue 11 is Y, residue 14 is S, T, or D or wherein residue 14 is S or T, and residues 1, 5. 7, 9, and 13 are hydrophobic residues or G;
X7 is a beta turn consisting of two amino acids in length;
Z7 is a beta strand consisting of 9 amino acid residues, wherein residue 8 is G, residues 7 and 9 is W or Y, and residues 1, 3, and 5 are hydrophobic residues or G;
X8 is a loop comprising at least 5 amino acids;
Z8 is a beta strand consisting of 12 amino acid residues, wherein residue 1 is N or Q, residue 6 is G, residue 9 is Y, and residues 1, 3, 5, 7, and 11 are hydrophobic residues or G.

As described in detail herein, the proteins of the disclosure are eight stranded transmembrane (TMB) proteins that insert and fold into detergent micelles and synthetic lipid membranes. The designed proteins fold more rapidly and reversibly in lipid membranes than the TMB domain of the model native proteins. Extensive data is provided defining the domain structure of the proteins as claimed.

X1 comprises at least 2 amino acid residues wherein the C-terminal residue in X1 is G, and may be of any length and amino acid composition so long as the C-terminal residue is G. As noted herein, X1 may comprise one or more added functional domains. In various embodiments, the C-terminal residues in X1 are PG or QG, or the C-terminal residues in X1 are PG.

Z1 is a beta strand consisting of 10 amino acid residues, wherein residue 1 is S. T or D. residue 9 is G and residue 10 is W or Y, and wherein residues 2, 4, 6, and 8 are hydrophobic residues or G. The other residues in Z1(residues 3, 5, and 7) may be any amino acid. In one embodiment, residue 1 in Z1 is S or T. In another embodiment, Z1 residue 5 is Y, Z5 residue 4 is Y, or both.

X2, X4, X6, and X8 are loops comprising at least 5 amino acids. Each of X2, X4, X6, and X8 may independently be of any length and amino acid composition. As noted herein, each of X2, X4, X6, and X8 may comprise one or more added functional domains. In certain embodiments, wherein none of X2, X4, X6, or X8 comprise (consecutively) the amino acid residues across a single row of Table 1.

TABLE 1

| | | | | |
|---|---|---|---|---|
| Pos1 | Pos2 | Pos3 | Pos4 | Pos5 |
| D | P | D | G | K |
| N | A | N | | N |
| T | S | A | | T |
| S | D | E | | S |
| | E | | | |

In other embodiments, X2, X4, X6, or X8 each independently comprise an amino acid sequence selected from the group consisting of the amino acid sequence of SEQ ID NOS:22-26.

NTDNT (SEQ ID NO:22)

NNSSL (SEQ ID NO:23)

TGQSG (SEQ ID NO:24)

DSWNK (SEQ ID NO:25)

ARONWNYIP (SEQ ID NO:26)

In another embodiment, residue 2 of X2 is Y.

X3, X5, and X7 are each a beta turn consisting of two amino acids in length. Each residue of X3, X5, and X7 may be any amino acid. In various embodiments, X3, X5, and X7 independently have P, E, or D at residue 1; and N, G, E, D, Q, or Y at position 2.

Z2 is a beta strand consisting of 12 amino acid residues, wherein residues 5 and 6 are G, residue 9 is Y, residue 12 is S, T, or D or wherein residue 12 is S or T, and residues 1, 3, 7, and 11 are hydrophobic residues or G. The other residues in Z2 (residues 2, 4, 8, and 10) may be any amino acid.

Z3 is a beta strand consisting of 9 amino acid residues, wherein residues 6 and 8 are G. residues 7 and 9 are W or Y, and residues 1, 3 and 5 are hydrophobic residues or G. The other residues in Z3 (residues 2 and 4) may be any amino acid.

Z4 is a beta strand consisting of 14 amino acid residues, wherein residue 1 is N or Q, residues 6-8 are G, residue 11 is Y, residue 14 is S, T, or D or wherein residue 14 is S or T, and residues 3, 5, 9, and 13 are hydrophobic residues or G. The other residues in Z4 (residues 2, 4, 10, and 12) may be any amino acid.

Z5 is a beta strand consisting of 11 amino acid residues, wherein residue 3 is P, residue 8 is G, residue 11 is Y or W, and residues 1, 5, 7, and 9 are hydrophobic residues or G. The other residues in Z5 (residues 2, 4, 6, and 10) may be any amino acid.

Z6 is a beta strand consisting of 14 amino acid residues, wherein residue 3 is P, residues 6 and 8 are G, residue 11 is Y, residue 14 is S, T. or D or wherein residue 14 is S or T, and residues 1, 5, 7, 9, and 13 are hydrophobic residues or G. The other residues in Z6 (residues 2, 4, 10, and 12) may be any amino acid.

Z7 is a beta strand consisting of 9 amino acid residues, wherein residue 8 is G, residues 7 and 9 is W or Y, and residues 1, 3, and 5 are hydrophobic residues or G. The other residues in Z7 (residues 2, 4, and 6) may be any amino acid.

Z8 is a beta strand consisting of 12 amino acid residues, wherein residue 1 is N or Q, residue 6 is G, residue 9 is Y, and residues 1, 3, 5, 7, and 11 are hydrophobic residues or G. The other residues in Z8 (residues 2, 4, 8, 10, and 12) may be any amino acid.

In various embodiments, one or more of the following is true:
Z1 residue 8 is A;
Z3 residue 5 is A;
Z5 residue 7 is A:
Z6 residue 5 and residue 7 are A or G; and/or
Z8 residue 5 is A or G.

In other embodiments, one or both of the following is true:
Z3 residue 4 is E or D and Z1 residue 5 is Y; and/or
Z7 residue 6 is E or D and Z5 residue 4 is Y.

The proteins of the disclosure may further comprise one or more functional domains. In one embodiment, one or more of X1, X2, X4, X6, and X8 comprise an added functional domain. In one embodiment, the protein comprises an added functional domain C-terminal to Z8; in another embodiment the protein comprises an added functional domain at the N-terminus. As used herein, a “functional domain” is any polypeptide of interest that might be fused or covalently bound to the proteins of the disclosure. In one embodiment, the one or more functional domains is present as a genetic fusion with the proteins of the disclosure. In non-limiting embodiments, such functional domains may comprise one or more polypeptide antigens, polypeptide therapeutics, enzymes, detectable domains (ex: fluorescent proteins or fragments thereof), DNA binding proteins, transcription factors, etc., for uses as described herein.

In another embodiment, the proteins comprise the amino acid sequence at least 50%, 55%, 60%, 65%, 70%, 75%, 80%, 85%, 90%, 95%, or 100% identical to the amino acid sequence selected from SEQ ID NOS:1-19, wherein residues in parentheses are optional and may be present or absent. In one embodiment, the optional residues are absent and are not considered when determining percent identity. In another embodiment, the optional residues are present and are considered when determining percent identify. Sequences of SEQ ID NO:1-19 are shown below, and position of residues in beta strands is shown below SEQ ID NO:19.

TABLE 2

Exemplary proteins

| | |
|---|---|
| Design name | Amino acid sequence |
| TMB2.3 | (MQDG) PGTLDVFVAAGWNTDNTIEITGGATYQLSPYIMVKAGYGWNNSSLNRFEFGGGLQYKVTPDL EPYAWAGATYNTDNTLVPAAGAGFRYKVSPEVKLVVEYGWNNSSLQFLQAGLSYRIQ(P) (SEQ ID NO:1) |
| TMB2.17 | (MEQK) PGTLMVYVVVGYNTONTVDYVEGAQYAVSPYLFLDVGYGWNINSSLNFLEVGGGVSYKVSPDL EPYVKAGFEYNTONTIKPTAGAGALYRVSPNLALMVEYGWNNSSLOXVAIGIAYKVK (D) (SEQ ID NO:2) |
| TMB2.24 | (MGQ) PQGSIAVSVELGYNTDNTISIVGGLSYALSPYLTVRAGYGWNNSSLNELVIGGGIFYQVSPEV EPYIAFGAKFNTDNTLKPFAGAGAAYKVSPELQLVAEYGNNNSSLQEIHVGFEYKLA (E) (SEQ ID NO:3) |
| TMB2.27 | (MGTK) PGSVWVKVLAGWNTDNTIVFSGGASYALTPYLEIEAGYGWNNSSLNAAFFGGGVMYTVSPDL EPYVWAGAHYNTDNTLKPAAGAGAKYRVTPDFALEARYGWNNSSLQVVEAGVTYKVK (D) (SEQ ID NO:4) |
| TMB2.31 | (MPSR) PGDLKVYLVAGWNTDNTIRFEGGLRYDVSPYLLLDAGYGWNNSSLNFLKVGGGFAYTLSPDI APYVLAGATYNTDNTLAPFAGAGFEYRLTPDLAAVIEYGWNNSSLQWLVAGVAYKVK (E) (SEQ ID NO:5) |
| TMB2.35 | (MSDK) PGSVALTLDIGWIIT DNTVDLVGGAVYALSPYLFLEAGYGWNNSSLNVIKFGGGIMYTLSPDL EPYVRVGAKINTDNTLKPEAGAGFFYKLTPDLKLKIDYGWNNSSLQTAAVGVTYKVQ (P) (SEQ ID NO:6) |
| TMB2.37 | (MGPK) PGSVYLVVEVGYNTDNTFELVGGLMYALSPYLTLSAGYGWNNSSLNTGKVGGGFYYQITPDL EPYVVVGFKFNTDNTVKPSAGAGALYRVSPDVVLRVEYGHNNSSLOVASVGIEYKVK (2) (SEQ ID NO:7) |
| TMB2.43 | (MTPK)PGSIALLVKVGYNTDNTIRFAGGAMYAVSPYVFVSAGYGWNNSSLNEFEFGGGVSYDLSPEL EPYVFAGATYNTONTIKPFFGAGEFYRVSPEVKGRVEYGWNNSSLOQEVAAGLVYKVC (G) (SEQ ID NO:8) |
| TMB2.45 | (MGQQ) PGTVRVFLVAGYNTDNTIVVMGGLQYAVSPYVALEAGYGWNNSSLNFLVIGGGLEYDVSPDI EPYVSLGFMYNTDNTIKPVIGAGAEYRLSPNLAVRIEYGWNNSSLQFVVAGLAYDVQ (K) (SEQ ID NO:9) |
| TMB2.47 | (MPDK) PGSVQLYVKVGYNTDNTLALEGGLDYAVSPYVFLDVGYGWNNSSLNEFVVGGGAKYTLSPEL EPYVFAGCVKYNTDNTLKPFACAGAEYRVSPNVKLRIEYGWRNSSLOVLAAGLAYKVR (D) (SEQ ID NO:10) |
| TMB2.58 | (MGQK) PGSIALFVVAGWNTDNTVELSGGLQYEVSPYVTVDAGYGWNNSSLNFFEAGGGVKYRVTPQL EPYVVAGVRYNTDNTLKPTAGAGAEYKLSPDLALRVEYGWNNSSLQFLRGGLKYQVK (D) (SEQ ID NO:11) |
| TMB2.60 | (MPEP) PGTVAIVVMVGYNTDNTFDVHGGLSYVLSPYLLVDAGYGWNNSSLNMVHVGGGVQYSGDPDL DPYLTAGVKYNTDNTLKPFAGAGFKYRVTPDLVIRVEYGWNNSSLQEAKVGFEYKLR (G) (SEQ ID NO: 12) |
| TMB2. 69 | (MRPQ) PGSVSVFLAAGWNTDNTIVIVGGASYKLSPYLELTAGYGWNNSSLNEIEVGGGVEYQLTPEI YPYVEAGAVYNTDNTLRPTAGAGAKYKLSPNLALRADYGNNNSSLQKVKAGVEYTLI (P) (SEQ ID NO:13) |
| TMB2.70 | (MGPK) PGSLELYVVAGWNTDNTIELKGGLOYAISPYLSLDVGYGWNNSSLNKFEAGGGLEYRLTPEI VPYVKRGLSWNTDNTVKPARGAGAKYKLSPDLALMIEYGHNNSSLNWLVAGASYKIK (D) (SEQ ID NO:14) |
| TMB2.71 | (MQPV) PGSVFITVAIGYNTDNTLKIMGGLEYVVSPYGSVVAGYGWNNSSLNEIKVGGGLHYKLSPDI FPYVVAGVVYNTDNTLKPTAGGGVLYKLSPELFARVEYGWNNSSLQEVLVGAAYRVR (P) (SEQ ID NO:15) |
| TMB2.73 | (MPFK) PGSVEVYVAGGWNTDNTIVIKGGLQYAVSPYFALDVGYGWNNSSLNTGMAGGGFLYVVTPDL EPFVSGGVKFNTDNTAKPMVGAGFTYRLSPNLALRVWYGWNNSSLNEVEAGVSYRVK (D) (SEQ ID NO:16) |
| TMB2.75 | (MODK) PGTIRIVVMVGYNTDNTVDVSGGLTYALSPYLKITVGYGWNNSSLNLFEVGGGVEYTISPEV EPYVVAGVKYNTDNTLKPFAGAGFMYRLSPDLAAMVDYGWNNSSLNLARLGFAYKVQ (D) (SEQ ID NO:17) |
| THB2.81 | (MQKR) PGSVAAFVVAGWNTDNTLHLMGGAEYMLTPYLALRAGYGWNNSSLNTGKAGGGVKYKITPNL EPYIVAGVKWNTDNTVKPFAGAGFDYWLSPNLAITVEYGWNNSSLNEIEAGLSYEVK (S) (SEQ ID NO:18) |
| TMB2.83 | (MGTK) PGSFALAVAAGWNTDNTIVLVGGIRYSLSPYLFIEAGYGWNNSSLNFLFAGGGVSYQLSPDL EPYAAAGFLYNTDNTIAPWAGAGAKYRLTPDLEADVFYGWNNSSLQFIVAGLEYDVK (P) (SEQ ID NO:19) |
| Beta strands | SSSSSSSSSS SSSSSSSSS35S SSSSSSSSS SSS3SSSSSSSSSS SSSSSSSSSSS SSSSSSSSSSSS5S SSSSSSSSS SSSSSSSSSSSS |

In another embodiment, the proteins comprise an amino acid sequence at least 50%, 55%, 60%, 65%, 70%, 75%, 80%, 85%, 90%, 95%, or 100% identical to the amino acid sequence selected from SEQ ID NOS:20-21.

TMB2.3.long

(M) QDGPGTLDVFVAAGWNQYHDTGFINNNGPTHENKIEITGGATYQLS
PYIMVKAGYGWDGRMPYKGSVENGAYKRNPFEFGGGLOYKVTPDLEPYAW
AGATYERADTKSNVYGKNHDNKLVPAAGAGFRYKVSPEVKLVVEYGWKNN
IGDARTIGTRPDKQFLQAGLSYRIQP (SEQ ID NO:20)

TMB2.17.long

(M) EQKPGTLMVYVVVGYEQYHDTGFINNNGPTHENKVDVVGGAQYAVS
PYLFLDVGYGWTGRMPYKGSVENGAYKKNFLEVGGGVSYKVSPDLEPYVK
AGFEYERADTESNVYGKNHDNRIKPTAGAGALYRVSPNLALMVEYGWKNN
IGDAHTIGTRPDKQKVAIGIAYKVKD (SEQ ID NO:21)

In one embodiment, the N-terminal M residue in SEQ ID NO:20 and 21 is absent and not considered when determining percent identity. In another embodiment, the N-terminal M residue in SEQ ID NO:20 and 21 is present and is considered when determining percent identity.

The proteins can tolerate significant substitutions in undefined residue positions. In some embodiments, a given amino acid can be replaced by a residue having similar physiochemical characteristics, e.g., substituting one aliphatic residue for another (such as Ile. Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe, Non-conservative substitutions will entail exchanging amember of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp: and/or Phe into Val, into Ile or into Leu.

In all of these embodiments, the percent identity requirement does not include any additional functional domain that may be incorporated in the polypeptide. In non-limiting embodiments, such functional domains may comprise one or more polypeptide antigens, polypeptide therapeutics, enzymes, detectable domains (ex: fluorescent proteins or fragments thereof), DNA binding proteins, transcription factors, etc.

In another aspect, the disclosure provides proteins comprising the amino acid sequence at least 50%, 55%, 60%, 65%, 70%, 75%, 80%, 85%, 90%, 95%, or 100% identical to the amino acid sequence selected from SEQ ID NOS:1-21, wherein residues in parentheses are optional and may be present or absent. In one embodiment, the optional residues are absent and are not considered when determining percent identity. In another embodiment, the optional residues are present and are considered when determining percent identity.

In a further aspect, the disclosure provides non-naturally occurring, self-complementing multipartite beta barrel protein, comprising at least a first polypeptide component and a second polypeptide component, wherein the at least first polypeptide component and the second polypeptide component are not covalently linked, wherein in total the at least first polypeptide component and the second polypeptide component comprise domains X1-Z1-X2-Z2-X3-Z3-X4-Z4-X5-Z5-X6-Z6-X7-Z7-X8-Z8, wherein each domain is as defined herein according to any embodiment or combination of embodiments;

wherein (a) each beta strand (Z1-Z8) is fully present within one polypeptide component of the at least first polypeptide component and the second polypeptide component, (b) none of the at least first polypeptide component and the second polypeptide component include each of Z1, Z2, Z3, Z4, Z5, Z6, Z7, and Z8; and (c) one of domains X2, X4, X6, and X8 may be partially or wholly absent in each of the first polypeptide and the second polypeptide.

The split proteins comprise at least a first polypeptide component and a second polypeptide component in which β-strands are preserved while split points in the β-barrel proteins are taken only in the loops. In other words, each beta strand or (Z1, Z2, Z3, Z4, Z5, Z6, Z7, and Z8) is fully present within one polypeptide component of the at least first polypeptide component and the second polypeptide component, while the β-barrel polypeptide is split into separate components at loops (X2. X4, X6, and X8). By way of non-limiting example, in various embodiments of a bipartite β-barrel protein, the first polypeptide component and the second polypeptide component may comprise components as exemplified in Table 3.

TABLE 3

| | | |
|---|---|---|
| Example | First polypeptide component comprises | Second polypeptide component comprises |
| 1: Split at X2 loop | X1-Z1-(X2) | (X2)-Z2-X3-Z3-X4-Z4-X5-Z5-X6-Z6-X7-Z7-X8-Z8 |
| 2: Split at X4 loop | X1-Z2-X2-Z2-X3-Z3-(X4) | (X4)-Z4-X5-Z5-X6-Z6-X7-Z7-X8-Z8 |
| 3: Split at X6 loop | X1-Z2-X2-Z2-X3-Z3-X4-Z4-X5-Z5-(X6) | (X6)-Z6-X7-Z7-X8-Z8 |
| 4: Split at X8 loop | X1-Z2-X2-Z2-X3-Z3-X4-Z4-X5-Z5-X6-Z6-X7-Z7-(X8) | (X8)-Z8 |

As used throughout the present application, the term “polypeptide”, “peptide”, and “protein” are used interchangeably in their broadest sense to refer to a sequence of subunit amino acids of any length, which can include genetically coded and non-genetically coded amino acids, chemically or biochemically modified or derivatized amino acids, and polypeptides having modified peptide backbones. The proteins of the disclosure may comprise L-amino acids + glycine, D-amino acids + glycine (which are resistant to L-amino acid-specific proteases in vivo), or a combination of D- and L-amino acids + glycine. The proteins described herein may be chemically synthesized or recombinantly expressed. The proteins may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In another aspect, the disclosure provides nucleic acids encoding the beta barrel protein or the first or second polypeptide of any embodiment described herein. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, outer membrane localization and/or insertion signals, and plasma membrane localization signals. It will be apparent to those of skill in the art, based on the teachings herein, what nucleic acid sequences will encode the proteins of the disclosure.

In a further aspect, the disclosure provides expression vectors comprising nucleic acids of the disclosure operatively linked to a control sequence. “Expression vector” includes vectors that operatively link a nucleic acid coding region or gene to any control sequences capable of effecting expression of the gene product. “Control sequences” operatively linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In one aspect, the disclosure provides recombinant host cell comprising the proteins, polypeptide components, nucleic acids and/or the expression vectors of any embodiment or combination of embodiments of the disclosure. The host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R.I. Freshney. 1987. Liss, Inc. New York, NY)). A method of producing a protein according to the invention is an additional part of the invention. The method comprises the steps of (a) culturing a host according to this aspect of the invention under conditions conducive to the expression of the protein, and (b) optionally, recovering the expressed protein. The expressed protein can be recovered from the cell free extract, but preferably they are recovered from the culture medium, and (c) optionally, reconstitute the protein in vitro in detergent micelles or lipids.

The disclosure further provides pharmaceutical compositions, comprising
(a) the beta barrel protein, self-complementing multipartite beta barrel protein, first polypeptide, second polypeptide, nucleic acid, expression vector, and/or recombinant host cell of any embodiment herein; and
(b) a pharmaceutically acceptable carrier.

The pharmaceutical compositions of the disclosure can be used, for example, in the methods of the disclosure described herein. The pharmaceutical carrier may comprise, for example, a lipid-based compartment, including but not limited to liposomes, uni-lamellar vesicles, micelles, etc. The pharmaceutical composition may further comprise any other components as deemed appropriate for an intended use.

The disclosure also provides methods for using the beta barrel proteins, self-complementing multipartite beta barrel proteins, first polypeptide, second polypeptide, nucleic acid, expression vector, recombinant host cell and/or pharmaceutical composition of any embodiment herein, for uses including, but not limited for scaffolding binding epitopes and functional domains on liposomes, cell surface, or detergent micelles, for drug delivery, and as ion, water or small-molecule permeable transmembrane channels. Such uses are discussed in the examples that follow.

The disclosure further provides methods for designing beta barrel proteins or components thereof, comprising any embodiment or combination of embodiments of protein design steps disclosed herein. Such design methods are described in detail in the examples that follow.

### EXAMPLES

Here we leverage the power of de novo computational design to determine principles underlying transmembrane β-barrel proteins (TMB) structure and folding, and find that, unlike almost all other classes of protein, locally destabilizing sequences in both the β-tums and β-strands facilitate TMB expression and global folding by modulating the kinetics of folding and the competition between soluble misfolding and proper folding into the lipid bilayer. We use these principles to design new eight stranded TMBs with sequences unrelated to any known TMB and show that they insert and fold into detergent micelles and synthetic lipid membranes. The designed proteins fold more rapidly and reversibly in lipid membranes than the TMB domain of the model native protein OmpA, and high resolution NMR and X-ray crystal structures are very close to the computational model.

The de novo design of an integral transmembrane β-barrel (TMB) has not yet been achieved. TMBs can spontaneously fold into lipid bilayers from an unfolded chain, possibly through a mechanism involving concerted membrane insertion and folding of the β-hairpins. How this folding in a non-aqueous environment is encoded in the sequences of TMBs is not well understood because of experimental challenges in characterizing the rugged folding pathway — including possible off-pathway, misfolded or “invisible” states — and the often non-superimposable folding and unfolding equilibria (hysteresis).

To shed light on the sequence determinants of folding and stability of TMBs, and to enable the custom design of TMBs for specific applications, we set out to design TMBs de novo. We started by studying the constraints membrane embedding puts on both the backbone geometry and sequence of β-barrels.

### Geometric Constraints on Transmembrane Β-Barrel Backbones

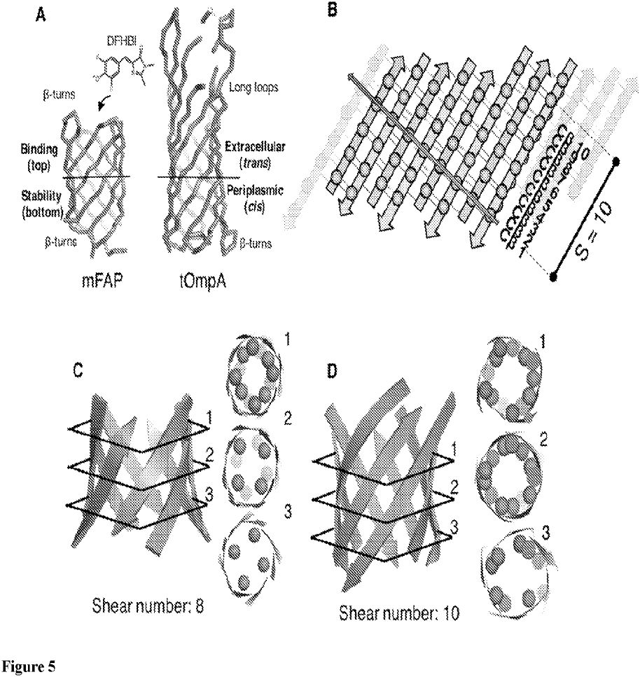

TMBs are formed from a single β-sheet that twists and bends to close on itself, so that all membrane-embedded backbone polar groups are hydrogen-bonded and shielded from the lipid environment. Insertion of TMBs into the lipid membrane is oriented (17), with β-strands usually connected with long loops on the translocating (trans) side of the β-barrel (extracellular in bacteria) and short β-turns on the non-translocating (cis) (FIG. 5A). The βbarrel architecture is characterized by two discrete parameters: the number of strands (n) and the shear number (S)--the shift in the number of residues (register shift) along a strand after tracing around the barrel through the backbone hydrogen bonds (18). The ideal β-barrel radius r (eq. 1) and angle of the strands with the main barrel axis θ(eq. 2) are functions of n, S, the average distance between two β-strands (D) and the average distance between two residues on a β-strand (d) (Table 4) (19).

$$r = {\left[{\left(S d\right)}^{2} + {\left(n D\right)}^{2}\right]}^{1 / 2} / \left[2 n s i n \left(\frac{π}{n}\right)\right] \qquad \text{eq. 1}$$

$$t a n \left(θ\right) = \frac{S d}{n D} \qquad \text{eq. 2}$$

The shear number (S) and the number of strands (n) also define the packing arrangement of the stripes of Cβs packing along the interstrand hydrogen bonds (half of the Cβ-stripes point toward the β-barrel lumen and the other half toward the β-barrel exterior) (FIG. 5B).

TABLE 4

Number of Cβ-strips, ideal values of β-barrel circumference based on the average radius and strand staggering angle for the identified classes of β-barrels

| Number of strands (n) | Shear number (S) | R (Å) | θ (°) | Number of core Cβ-strips |
|---|---|---|---|---|
| 8 | 8 | 7.3 | 36.3 | 4 |
| 8 | 10 | 8.0 | 42.5 | 5 |
| 10 | 12 | 9.7 | 41.3 | 6 |
| 12 | 12 | 10.8 | 36.3 | 6 |
| 12 | 14 | 11.4 | 40.5 | 7 |
| 12 | 16 | 12.2 | 44.4 | 8 |
| 12 | 18 | 12.9 | 47.7 | 9 |
| 12 | 24 | 15.4 | 55.7 | 12 |
| 14 | 14 | 12.5 | 36.3 | 7 |
| 14 | 16 | 13.2 | 40.0 | 8 |
| 16 | 16 | 14.3 | 36.3 | 8 |
| 16 | 18 | 15.0 | 39.5 | 9 |
| 16 | 20 | 15.6 | 42.5 | 10 |
| 16 | 22 | 16.4 | 45.2 | 11 |
| 18 | 18 | 16.1 | 36.3 | 9 |
| 18 | 20 | 16.7 | 39.2 | 10 |
| 18 | 22 | 17.4 | 41.9 | 11 |
| 19 | 20 | 17.3 | 37.7 | 10 |
| 22 | 24 | 20.2 | 38.7 | 12 |
| 24 | 26 | 22.0 | 38.5 | 13 |
| 26 | 30 | 24.5 | 40.2 | 15 |
| 36 | 18 | 27.5 | 20.1 | 9 |
| 60 | 60 | 53.3 | 36.3 | 30 |
| 108 | 54 | 32.4 | 20.1 | 27 |

The radius and strand staggering angle were calculated using equation 1 and equation 2 in the main text, which were reported in (19). The average distance between two Ca atoms along a βstrand is 3.3 Å and the average distance between two strands is 4.5 Å,

We chose to focus on the simplest and smallest β-barrel architecture of 8 β-strands. We first considered a shear number of 8 (n==S). In such a configuration, the total register shift is distributed equally among the four β-hairpins (2 residues per hairpin) and the side-chains pointing toward the lumen of the barrel are arranged into 4-fold symmetric Cβ-stripes (FIG. 5C). We found that such a symmetric packing arrangement combined with a small β-barrel radius does not allow tight jigsaw-puzzle like packing in the core as the Cα-Cβ vectors at each rung of the barrel point at each other. We chose to break the symmetry in the core by designing β-barrels with a shear number of 10; in this case the Cα-Cβ vectors are arranged into 5 intertwined Cβ-strips which spiral around the barrel axis so different side-chains are at different heights and more uniform packing can be achieved (FIG. 5D). To do so, we increased the register shift between two β-strands from 2 to 4 residues, which increases the barrel radius (eq. 1) and the angle of the β-strands with the barrel axis (eq. 2).

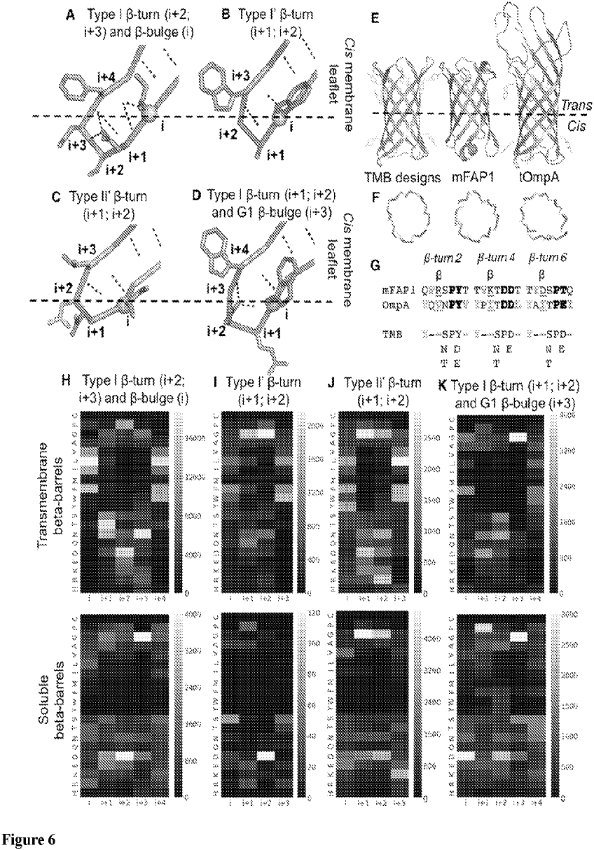

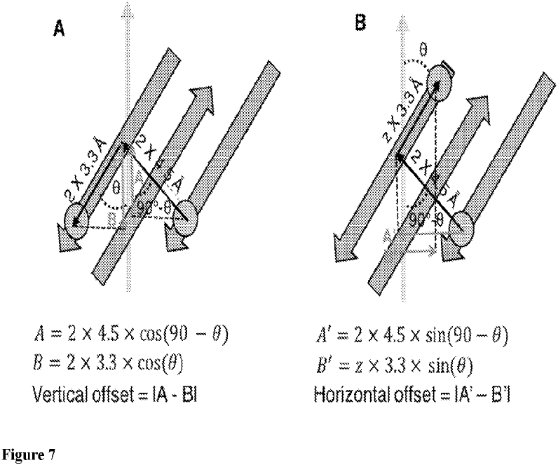

The uneven distribution of register shifts between hairpins complicates interactions with the lipid membrane. The bilayer can be approximated as two planes that must be parallel to ensure constant membrane thickness. In natural TMBs the cis (periplasmic) β-turns are close to the periplasmic lipid/water boundary (FIGS. 6A-D). While the β-turn residues closely match the sequence preferences observed in water-soluble β-barrels (mostly polar residues), the surface-exposed residues flanking these β-turns are predominantly hydrophobic (FIGS. 6H-K). We postulated that the hydrophobic residues upstream of the β-turns define the cis boundary of the transmembrane region because of their lowest position on the staggered hairpins (“membrane anchor residues”, FIGS. 6A-D). The geometric challenge is that the plane representing the cis membrane boundary must be aligned with the position of these four anchor residues in 3D space. Whereas the symmetric n=S=8 barrel has flat rungs which can be readily aligned with the membrane planes, the n=8 S=10 barrel does not. The total change of level (Z) between the lowest and the highest of the 4 anchor residues along the main β-barrel axis (eq. 5) is the sum of the difference in vertical offset along the β-strands (eq. 3, where a is the register shift) and along the hydrogen bonds (eq. 4, where b=2 is the number of strands between each anchor residue) (eq. 5, FIG. 7A).

$$A = a d c o s \left(θ\right) \qquad \text{eq. 3}$$

$$B = b D c o s \left(90 ° - θ\right) \qquad \text{eq. 4}$$

$$Z = {\sum }_{i = 1}^{n / 2} \left|A i - B i\right| \qquad \text{eq. 5}$$

$$C = {\sum }_{i = 1}^{n / 2} \left(a d c o s \left(90 ° - θ\right) + b D c o s \left(θ\right)\right) i \qquad \text{eq. 6}$$

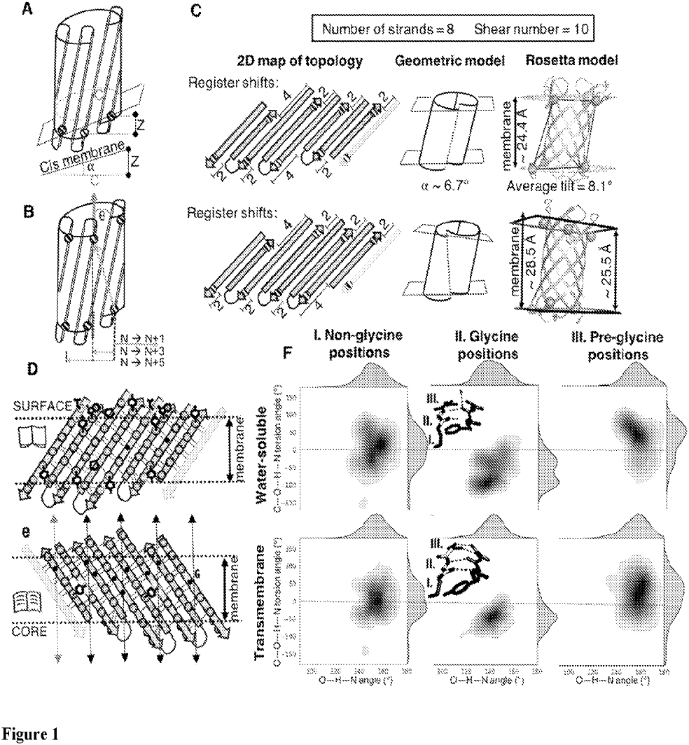

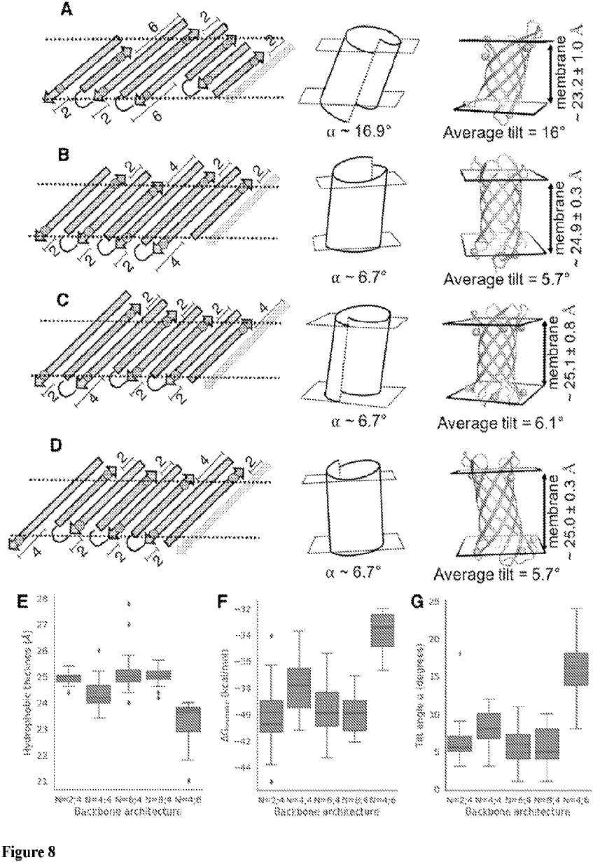

This vertical offset can in principle be accommodated by tilting the β-barrel by an angle α = arctan (Z/C) where the denominator is the length of the arc between anchor residues 1 to 4 projected onto the plane perpendicular to the main axis (eq. 6) (FIG. 1A, FIG. 7B). In the case of a β-barrel with symmetry (n=8, S=8), the vertical offset between each anchor residue is negligible (0.14 Å) and no tilt is required. When the shear number is increased to 10 by increasing the register shift between one pair of hairpins to 4 residues, the total vertical offset through the β-sheet is 3.9 Å over an arc length of 33.3 Å, and the barrel must be tilted by approximately 6,7° to the transmembrane axis (FIG. 1C, top). When the total 10 residue register shift is achieved instead by assigning a 6 residue register shift to one pair of hairpins, and zero shift to a second pair, eq. 5 and y6 predict a total vertical offset of 8.8 Å over 28.8 Å, and hence a more pronounced tilt angle α of approximately 16.9° (FIG. 8A). To validate this geometric model, we assembled sequence-agnostic protein backbones with the Rosetta® fragment assembly protocol (21), designed the lipid-exposed surface, and predicted the lipid membrane position with the PPM server (22). The average predicted tilt angles of the barrel to the transmembrane axis are close to the predictions for each of the register shift distributions considered above (8.1° (FIG. 1C, top) and 16°, respectively (FIGS. 8A,G)). We decided to continue our design efforts with the less tilted configuration, because it had a better match to the desired hydrophobic thickness of the membrane (24.3 Å+/-0.6) than the more tilted configuration (23.2 Å+/-1) (FIG. 8E) and had a more negative transfer energy from water to lipid (-38 kcal/mol vs -34 kcal/mol; predicted with the PPM server) (FIG. 8F)). Placing the four-residue register shift after any of the four cis hairpins resulted in structures with similar average hydrophobic thicknesses, tilt angle to the membrane axis and transfer energy from water to lipid and only differed on the direction of the tilt (FIGS. 8B-G); we chose to focus on one of these placements in which the 4-residue register shift is in the middle of the β-sheet.

We next investigated the structural consequences of the fact that the cis and trans planes representing the membrane boundary must be roughly parallel to each other to keep the thickness of the membrane constant. We reasoned that the two planes could only be kept parallel if the offset Z for any hairpin on the cis face is matched by a similar offset for the hairpin directly above it on the trans face (FIG. 1B). We determined the projection of the vector between a cis anchor residue and all four trans anchor residues for a β-barrel spanning a membrane of 24 Å (eq. 3) on the plane perpendicular to the main barrel axis; we consider the cis and trans anchor residue pairs with the smallest projected distance to stack on each other along the barrel axis. For barrel topologies of (n=8,S=10), we found that the stacking partner for an anchor residue on the cis side of strand N is the anchor residue on the trans side of strand N+3. Hence, to maintain constant thickness, the offset Z between strands N and N+1 on the cis side must be equal to the offset between strands N+3 and N+4 on the trans side. To confirm this prediction of our geometric model, we set the cis side register shift between strands N and N+1 to four residues, and ran Rosetta® design simulations and transmembrane plane predictions on backbones with a matching 4-residue register shift on the trans side between (i) strands N+3 and N+4 and (ii) strands N+5 and N+6. We averaged planes representing the membrane boundary in cis and trans and found, consistent with the model, parallel planes and constant hydrophobic thickness for the four residue register shift in N+3, but a 3 Å change in membrane thickness in the N+5 case (FIG. 1C, bottom).

### Sequence Design on Ideal Β-Barrel Backbones

We used this constant hydrophobic thickness constraint to guide the distribution of the register shifts around the β-barrel. The cis hairpins were closed with short β-turns associated with an upstream β-bulge (abundant in water-soluble and transmembrane β-barrels (FIG. 6A). Canonical β-turn sequences with strong β-hairpin nucleating properties (3:5 type 1 β-turns + G1 bulge with canonical SDG sequence) were used to connect the strands on the trans side in place of the long loops found in native TMBs; such turns were previously used to design water-soluble β-barrels (FIGS. 6E, G). To relieve strain from high β-sheet curvature, we placed glycine kinks (5) — glycine residues with an extended β-sheet backbone conformation — into the TMB backbone description (the backbone “blueprint”) such that a) every Cβ-strip pointing to the core of the barrel contains a glycine and b) there are no more than 4 non-glycine residues in a row in the Cβ-strips (¼ of the average barrel circumference). Following these principles, we designed a β-barrel blueprint in which the glycine kinks in the core of the protein were stacked along four vertical lines together with β-bulges associated with the cis hairpins (FIGS. 1D, E). Rosetta® models built from the above blueprint have four regions of strong β-sheet bending surrounding a wide β-barrel lumen (FIG. 6F).

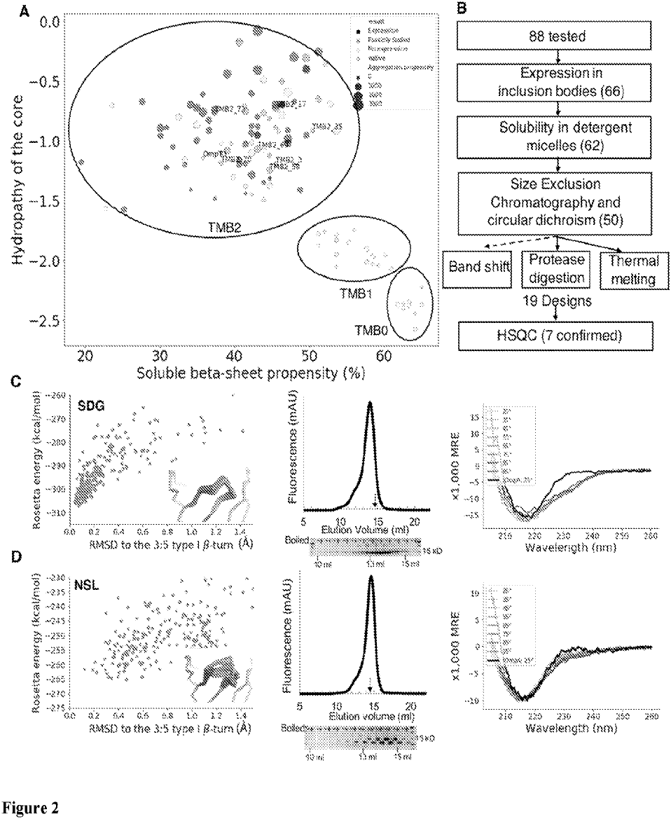

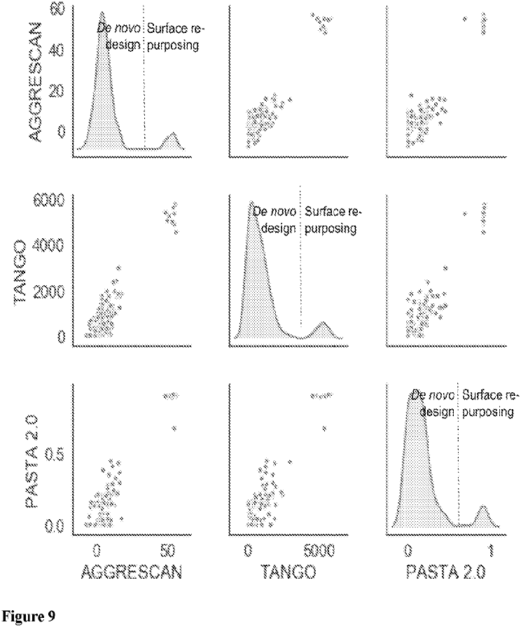

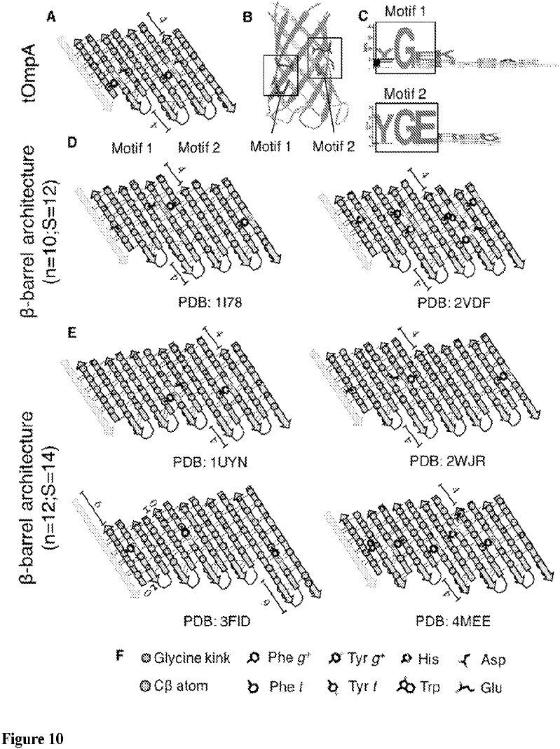

To delimit the upper and lower membrane boundaries, four tyrosine residues were placed two positions upstream of the anchor residues on the cis side, and alternating tyrosine and tyrosine/tryptophan motifs were placed at the trans boundary (FIG. 1D). To design the remainder of the sequence, we first considered the possibility that the core residues could be largely non-polar (helical transmembrane proteins have been designed by keeping the core of soluble designs fixed and resurfacing the outside with hydrophobic residues). However, this approach was rapidly dismissed as the resulting sequences had very strong amyloid propensity (FIG. 9). We next experimented with requiring the interior of the barrel to be polar to achieve the classic alternating hydrophobic-polar sequence pattern of canonical β-strands (inside/out model). We restricted the core positions to polar amino acids (excluding the glycine kink positions), increased the weight on the Rosetta® electrostatic potential to favor sidechain-sidechain hydrogen bonds, and restricted the surface to hydrophobic amino acids. To help define the register between β-strands we placed tyrosine residues adopting the +60,90 rotamer angles to closely interact with the grove formed by a hydrogen-bonded glycine kink partner and donating a hydrogen bond to a negatively charged residue (this is an extended definition of the mortise/tenon motif). Two positions were considered, the area of the sharp change of level between anchor residues (4-residues register shift) on the cis and trans faces, and the other side of the barrel between the first and last strands (FIG. 1E, FIG. 10). Finally, we designed full β-barrel sequences using Rosetta® combinatorial sequence design guided by these principles and found, as expected, that the secondary structure was accurately recapitulated by secondary structure prediction programs (FIG. 2A).

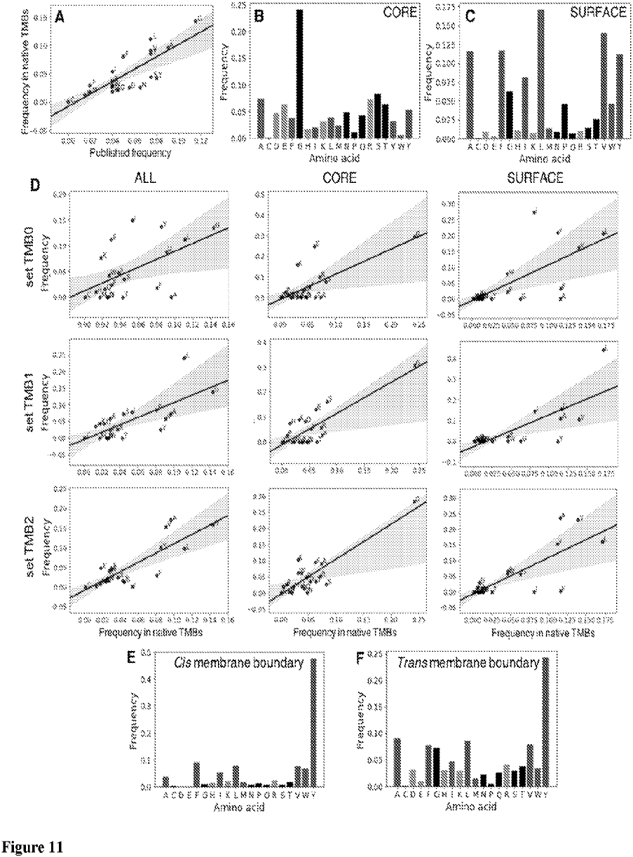

Folding of TMBs is chaperone-mediated and catalyzed in vivo (by the β-barrel assembly machinery (BAM) complex in Gram-negative bacteria, the sorting and assembly machinery (SAM) complex in mitochondria, and the translocase of the outer chloroplast membrane (TOC) complex in chloroplasts). Since it was unclear whether our TMB designs would be able to interact with the chaperone machinery to fold in the outer membrane of E. coli, we chose to express them in the cytoplasm, with the anticipation that the expressed sequences would form inclusion bodies that could then be solubilized in urca/guanidinium chloride. We obtained E. coli codon optimized synthetic genes for 9 designs (set TMB0, FIG. 11), but no protein of the correct molecular weight was produced upon the induction of protein expression. Reasoning that the designed sequences may have had too much positive charge, in a second round of 16 designs, we reduced the number of charged residues in the core of the protein (set TMB1, FIG. 11), but again none expressed in E coli.

These failures in expressing our TMB designs in E. coli were challenging because it was difficult to get feedback to improve the design methodology. To make progress, we took a step back and compared our designs to sequences of natural 8-strand TMBs. We noted two differences: first, the natural TMBs often have at least one of the trans loops disordered and greater than 20 residues in length, and second, the secondary structure propensity of the natural TMBs was lower than the designs we tried to express (FIG. 2A). We hypothesized that the strong secondary structure propensity of our designed sequences could result in folding of non-designed soluble β-sheet structures when expressed in the cytoplasm — possibly amyloid-like intermediates — which could be toxic and hence cleared rapidly and/or hindering growth of expressing cells.

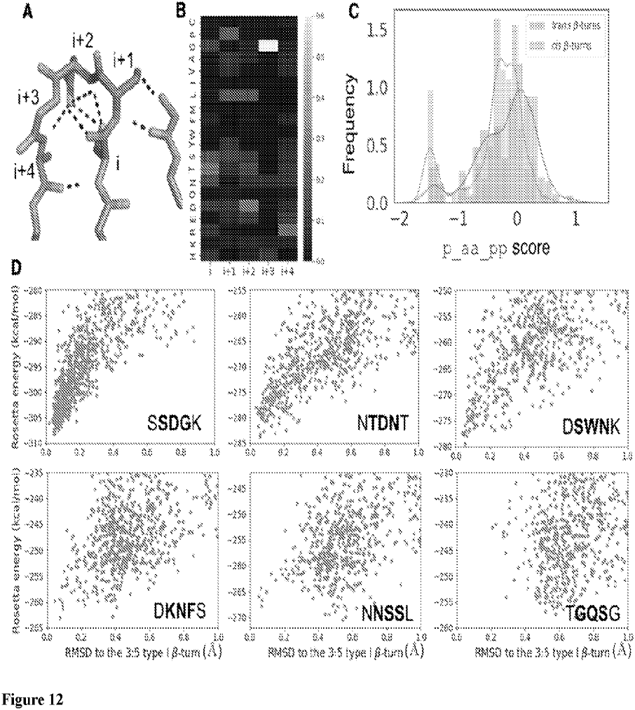

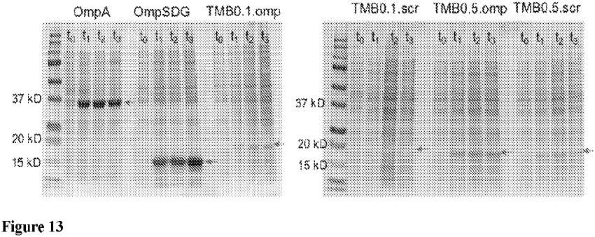

We first considered the possibility that the long disordered loops in trans might be necessary to slow down the non-native folding in the cytoplasm. To test this hypothesis, we obtained synthetic genes encoding 4 of the TMB0 designs with the extracellular loops replaced with either the extracellular loops of the native TMB domain of Outer Membrane Protein A of E. coli (tOmpA) or scrambled versions of these loops, as well as a redesigned version of tOmpA in which its trans loops were replaced with the 3-residues 3:5 type 1 β-turns used in our designs (with the canonical sequence SDG, FIGS. 12A,B). The re-looped tOmpA construct (OmpSDG) expressed at high levels in E. coli (where it was found in inclusion bodies), however, only two of the de novo designs with long loops showed expression but at very low levels which were insufficient for further characterization (FIG. 13). These results suggest that the protein expression failure is largely determined by the transmembrane β-strands rather than by their β-hairpin connections. Further characterization showed that the OmpSDG protein, while highly expressed, was not correctly folded: its circular dichroism (CD) spectrum, particularly in the 230 nm region (FIG. 2C), was different from native tOmpA when refolded in n-dodecyl-β-D-maltopyranoside (DDM) detergent at 2 times the critical micelle concentration (CMC), and it did not show the heat-modifiable band shift on SDS-PAGE characteristic of folded tOmpA (35) in DDM detergent and when refolded in large unilamellar vesicles (LUVs) of 1,2-diundecanoyl-sn-glycero-3-phosphocholine (DUPC, diC_{11;0}PC) (FIG. 2C).

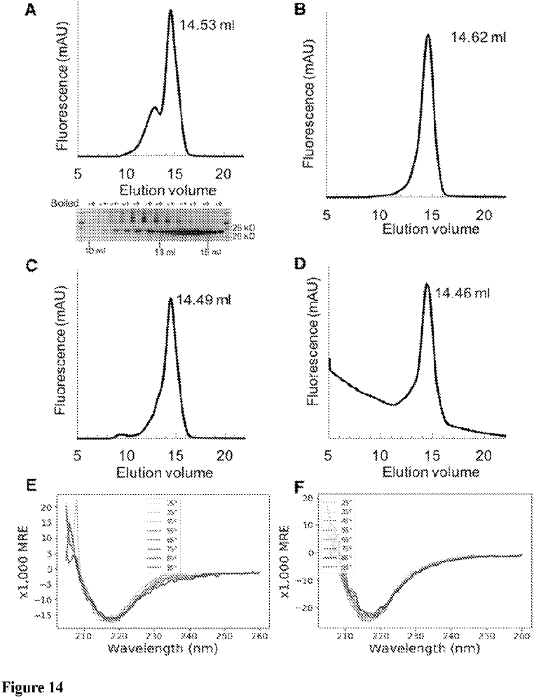

To understand the failure of OmpSDG to fold, we searched the PDB for short β-turns at the trans membrane boundary of natural TMB PDB structures, which are rare. We found five 3-residue trans β-turns whose backbone conformation and hydrogen bonding pattern satisfied all the characteristics of the canonical 3:5 type 1 β-turn with a G1 β-bulge. However, the sequences of these β-turns are suboptimal for their structure compared to the SDG canonical sequence, as shown by the structure/energy landscapes computed with Rosetta® for each of these turns (FIG. 2C, FIG. 12D). Further evidence of different properties for trans and cis β-turns despite identical backbone conformations in crystal structures is that small protein fragments retrieved from the PDB by matching sequences found in cis showed β-turn-like structural properties, while queries matching sequences of trans β-turns did not show any structural convergence. We tested whether this observation could be generalized by comparing sets of trans and cis β-turns of two to five residues and found worse predicted sequence-structure compatibility (Rosetta® p_aa_pp score) in trans turns (FIG. 12C). We hypothesized that, much like the long loops of native tOmpA, short non-canonical sequences could slow down nucleation of the trans β-hairpins. Accordingly, we tested 4 variants of tOmpA (OmpTrans1-4) that each contain two 3:5 type 1 β-turns with suboptimal sequences (these designs are shorter than the shortest variant of tOmpA previously reported, which has trans connections of 5 to 1 8residues). The proteins were again expressed at high levels in inclusion bodies (Table5 ), but this time all four of these sequences showed a heat-modifiable band in DDM detergent micelles and LUVs characteristic of a folded TMB (FIG. 2D). We selected one of the variants — OmpTrans3 — that appeared to be produced in the largest amounts for further characterization. OmpTrans3 refolded in detergent micelles had a similar retention time to native tOmpA on a Size Exclusion Chromatography (SEC) column (FIG. 2D, FIG. 14), a similar native mass spectrometry (nMS) profile well-dispersed resonance peaks by H¹-N¹⁵-HSQC NMR in Fos-choline-12 (DPC) detergent (data not shown) and a similar CD spectrum to tOmpA in DDM detergent (FIG. 2D) and in LUVs with the distinctive 231 nm peak. These data support the idea that slowed folding due to the presence of long or short suboptimal β-hairpin connection sequences on the trans side are necessary for proper folding of TMBs in vitro.

Guided by these results, we used the suboptimal β-turns we had inserted into the OmpTrans3 design in all of subsequent TMB de novo designs. To address the expression problem, we hypothesized that the culprit was the relatively high secondary structure propensity of the β-strands, and sought to address this by (i) increasing the hydrophobicity of the β-barrel lumen and thereby disrupting the strict alternation of polar and hydrophobic residues along the β-strands and (ii) introducing glycines in specific positions on the lipid-exposed surface. We experimented with extending the tyrosine-glycine motifs to include a negative charged Asp or Glu hydrogen bond acceptor to the tyrosine, using the Rosetta® HBNet protocol (39) to exhaustively search through all the possible positions. We kept such YGD/E networks fixed, and used Rosetta® combinatorial sequence optimization to design the remainder of the sequence. We allowed all 18 amino acids other than Cys and Pro in positions facing the core of the barrel and hydrophobic amino acids only on the lipid-exposed surface. The models were selected based on protein backbone quality (backbone torsion angles and hydrogen bonds) and the quality of the networks around each YGD/E motif (hydrogen bond potential, size, connectivity and robustness of the networks).

TABLE 5

Cytoplasmic protein expression yield obtained for native tOmpA and designed β-turn variants

| | tOmpA | OmpTrans1 | OmpTrans2 | OmpTrans3 | OmpTrans4 | OmpAAG |
|---|---|---|---|---|---|---|
| Yield (mg/L) | 128 | 40 | 44 | 88 | 51 | 61 |

The expressions of the six constructs were carried out in parallel in a single experiment. The given yields were calculated after cleaning the inclusion bodies and dissolving the protein in 8 M urea.

We compared the designed surface residue composition to that of native transmembrane barrels, and found that glycine (which destabilizes β-strands), while very rare in the corresponding region of water soluble β-barrels (we found only four such examples -three were buried in the midst of dimerization interfaces) and disallowed in the above designs, represents 6.3% of all amino acids on the lipid exposed surface of natural 8-strands TMBs (FIG. 11D). These surface glycine residues of TMBs often precede glycine kinks hydrogen-bonded with core polar network hot spots (such as the tyrosines in the mortise/tenon motifs) or are located between two glycine kinks. We inspected crystal structures of water-soluble and transmembrane β-barrels and found that, while most β-strand residues have canonical in-plane backbone hydrogen bonds (O—H—N angle ~ 160°; C—O—H—N dihedral ~ 0°) (42) and canonical Φ and Ψ torsion angles (FIG. 1F, left, FIGS. 14C, D), glycine kinks have more extended backbone conformation (positive Φ and/or negative Ψ torsion angles (data not shown). In water-soluble β-barrels, glycine kinks also have out-of-plane hydrogen bonds geometrics characteristic of a left-hand twist (O—H—N angle ~ 130°; C—O—H—N dihedral ~ -100°, FIG. 1F), while the surface residues preceding the glycine kink have more pronounced right-hand twist (C—O—H—N dihedral > 0°, FIG. 1F). Many backbone carbonyls in these out-of-plane hydrogen bond geometries were found to interact with a secondary hydrogen bond donor in the crystal structures (such as a water molecule or a surface residue side chain), but such hydrogen bonds would likely be disfavored in TMBs, in the absence of water available to interact with the exposed carbonyls. Indeed, TMBs have a smaller population of glycine kinks and pre-glycine hydrogen bonds significantly deviating from in-plane geometry (FIG. 1F). We hypothesized that glycines in positions preceding glycine kinks could allow more canonical hydrogen bonds by relieving backbone strain. We carried out a further round of design of the surface-exposed residues allowing glycine and increasing the weight on the Rosetta® long range hydrogen bond energy (which favors in-plane geometries). All resulting designs had two to three surface glycines (in average 5.6% of the surface amino acids). Two of the glycines were common to all designs (G26 associated with a mortise/tenon motif and G56 between glycine kinks G55 and G57).

After three iterations between core and surface design, the design calculations converged on roughly 30 distinct network architectures with overall amino acid composition similar to that of natural 8-strands TMBs (FIG. 11D). Codon optimized synthetic genes were obtained for several representatives of each core network architecture for a total of 90 designs (set TMB2). We also expressed 20 additional variants of these designs incorporating the extracellular loops of tOmpA to evaluate the effect of loop length on folding. One hundred and eight of these designs were tested, 80 were well expressed and were found exclusively in inclusion bodies (or 66 unique designs, excluding trans loop doublons). Notably, the same designs expressed poorly or did not express at all either with short ideal β-turns or long loops. The relatively high success rate in obtaining protein expression is in striking contrast to the earlier iterations described above in which no expression was observed.

### Characterization of Folding, Stability and Structure

To test the ability of the designs to stably fold to TMB structures in vitro, we followed procedures used to fold tOmpA and other natural TMBs (FIG. 2B). Briefly, the inclusion bodies were dissolved in 8 M urea and rapidly diluted into DDM, DPC or n-octyl-β-D-glucopyranoside (OG) detergents at 2X CMC. Out of the sixty-six expressed unique designs, sixty-two formed soluble species in such conditions. We purified the protein/detergent complexes by SEC and characterized the fifty designs which had a SEC retention volume expected for a monomeric TMB (similar to the 8-stranded tOmpA monomer and OmpTrans3) and a far-UV CD spectrum characteristic of a β-sheet protein. Surprisingly, the well-established band-shift assay on SDS-PAGE was un-informative for the identification of folded de novo designed TMBs. Instead, we found a good agreement between the resistance of a design to protease digestion and a β-sheet characteristic far-UV CD spectrum even at high temperature (up to 95° C.). Ten such designs were analyzed with ¹H-¹⁵N HSQC NMR in DPC detergent micelles, and seven had well dispersed chemical shifts profiles characteristic of a folded protein in this detergent. In total, designs satisfied the biochemical screening criteria, suggesting that they fold into a TMB structure.

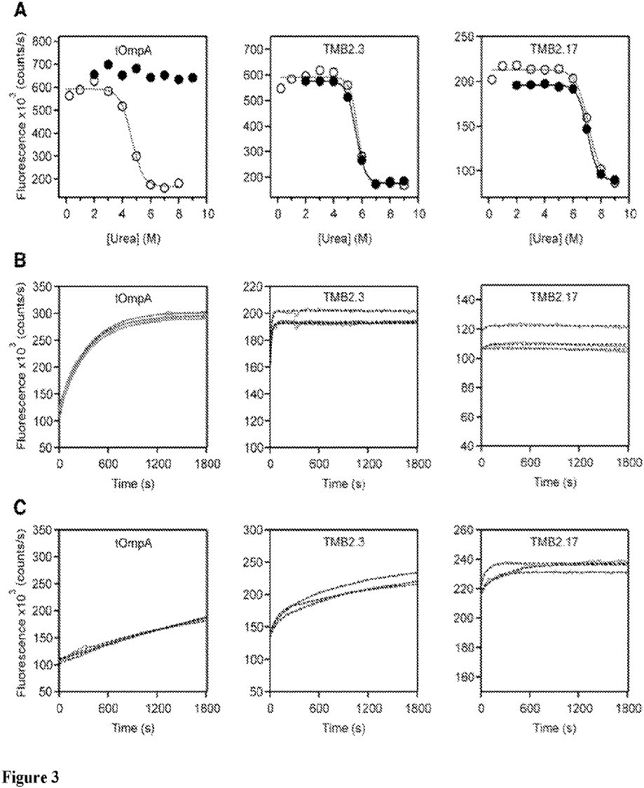

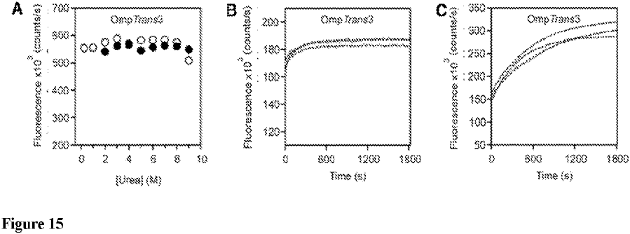

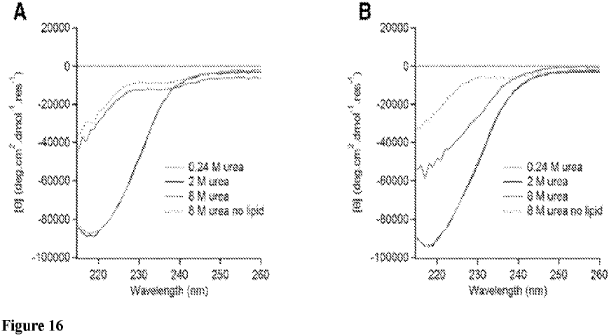

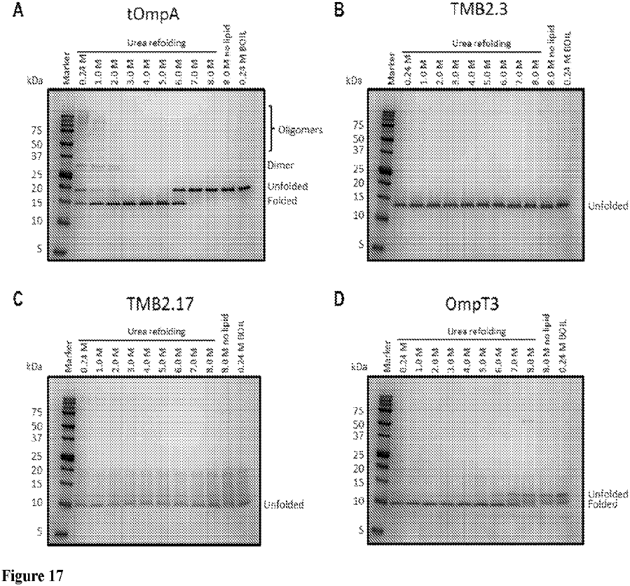

We selected two de novo designs (TMB2.17 (BLAST E-value to the non-redundant protein database: 0.10) and TMB2.3 (BLAST E-value: 0.035) and the OmpTrans3 construct for detailed biophysical characterization in a lipid bilayer to determine whether the proteins exhibit properties for a membrane spanning β-barrel (using tOmpA as a control for all our experiments). After refolding these four proteins into 100 nm DUPC LUVs, all proteins gave rise to far-UV CD spectra characteristic of a β-sheet both in 0.24 M and 2 M urea, and distinct from the spectra of the fully unfolded proteins in 8 M urea and from the proteins refolded in the absence of lipid (FIG. 16). We next determined the stability of the folded proteins by monitoring their ability to fold into/unfold out of LUVs at increasing urea concentrations, monitored by the change of fluorescence intensity between water-exposed and lipid embedded surface tryptophans (46). The results showed that the designed TMB proteins are more thermodynamically stable (midpoint urea concentration for folding (Cm^{F}) 5.7 M and 7.2 M for TMB2.3 and TMB2.17, respectively, FIG. 3A) than tOmpA (Cm^{F} = 4.7 M), while OmpTrans3 is the most stable protein as it appears folded in 9 M area (FIG. 15), in agreement with the far-UV CD data. It has been previously shown that many natural TMBs folding/unfolding transitions exhibit hysteresis due to the high kinetic barrier to unfolding and extraction from the membrane environment. Interestingly, in the conditions tested here, this behavior was observed for tOmpA but not for the designs TMB2.3 and TMB2.17 which showed superimposable and reversible unfolding/folding transitions, suggesting reduced kinetic stability relative to tOmpA. These observations likely explain the lack of a band-shift observed by SDS-PAGE, presumably since the lower kinetic stability causes the de novo designs to unfold during electrophoresis (FIG. 17).

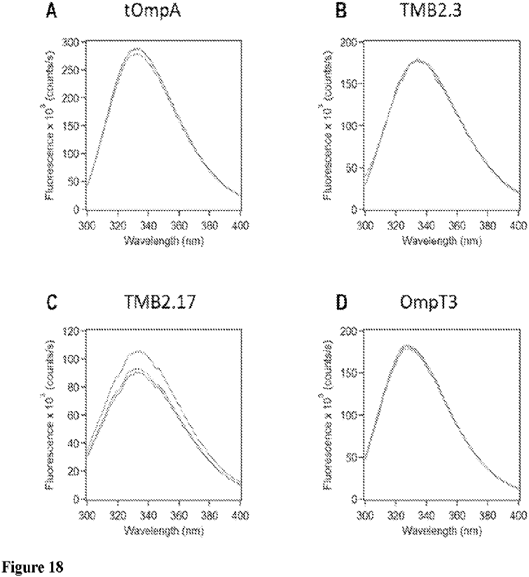

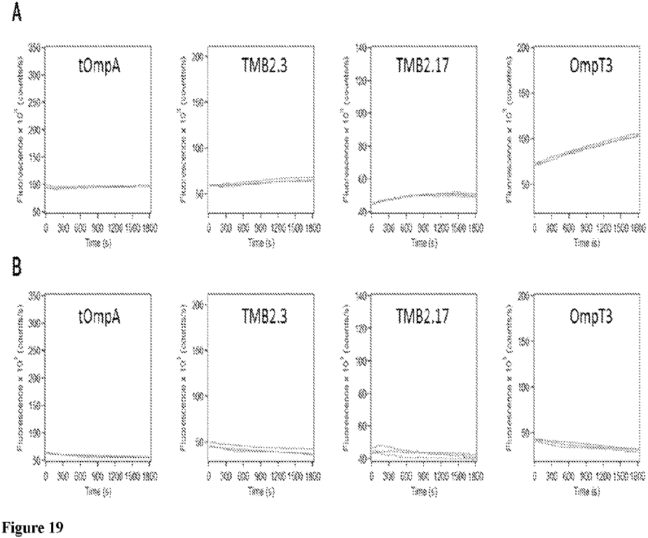

We next compared the kinetics of folding of the designed proteins to that of tOmpA (50) (FIG. 3B). These experiments showed that the designed TMBs fold over an order of magnitude more rapidly than tOmpA (folding rate constant of 3×10⁻³ s⁻¹ for tOmpA) under identical conditions: with a rate too rapid to allow accurate measurement of the folding rate constant. Tryptophan fluorescence emission spectra of the end point of the folding reactions confirm the TMBs were indeed fully folded (FIG. 18). Finally, to confirm that the designs integrate into the lipid bilayer rather than folding on the lipid surface or in the absence of lipid, proteins dissolved in 8 M urea were diluted into 2 M urea without lipid or into LUVs composed of 1,2-dimyristoyl-sn-glycero-3-phosphocholine (DMPC, diC_{14:0}PC). Consistent with previous results showing that the folding rates of natural TMBs are inversely correlated with lipid chain length, the designed TMBs fold more slowly into lipids of longer acyl chain length (FIG. 3C), and do not fold in the absence of lipid (FIG. 19B), confirming that they indeed integrate into the lipid bilayer upon completion of their folding.

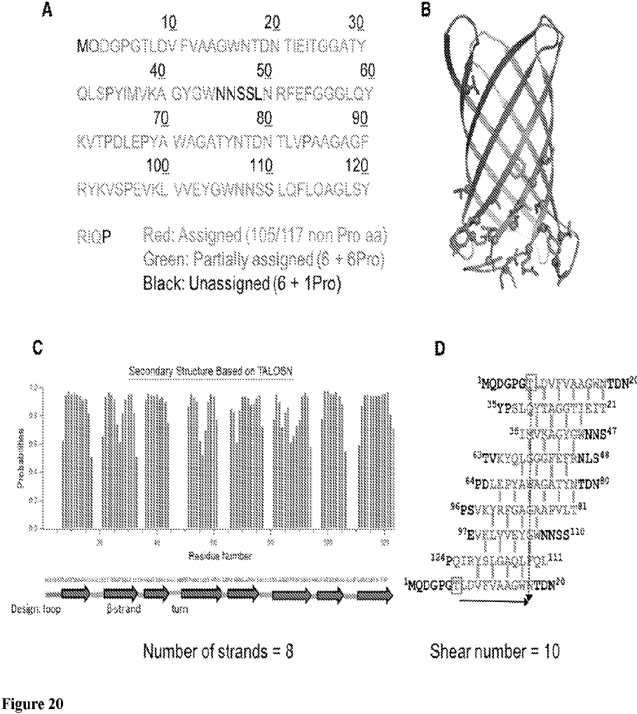

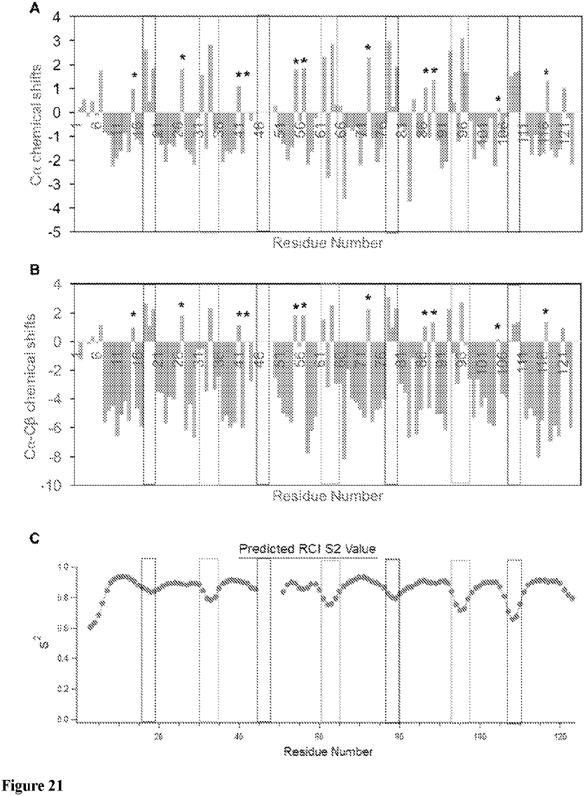

To characterize the structure of the designed TMBs in solution, we solved the structure of TMB2.3 folded into DPC detergent micelles using NMR spectroscopy (Table 6). Resonance peaks for 107 of the 117 non-proline residues of TMB2.3 were fully assigned: 6 more were partially assigned (FIG. 20A). Four out of six non-assigned residues were located in the trans β-turn regions - the remaining two were the N- and C-terminal residues of the protein. Analysis of the secondary structure content of TMB2.3, calculated using TALOS-N is consistent with 8 β-strands that closely match the β-strand boundaries in the designed model (FIG. 20C). 9 out of 11 glycine residues pointing toward the core of the β-barrel (glycine kink residues) have the designed torsional irregularities based on the positive Cα chemical shifts (FIGS. 21A, B) and the more extended predicted backbone conformations (Φ and Ψ closer to 180°). To validate the residue connectivity between the β-strands, we collected a total of 81 unique nuclear Overhauser Effects (NOEs) between amide protons; these suggest 72 inter-strand backbone hydrogen bonds that are in agreement with the β-strand connectivity of the design and the sheer number of 10 across the β-barrel (FIG. 20D). The NMR structure ensemble generated based on the chemical shifts and NOE information was in close agreement with the design model (average of 2.2 A RMSD,). We observed low-intensity additional resonance peaks for a subset of residues, indicating the presence of a (minor) secondary conformation. The secondary signals strong enough for analysis were consistent with the secondary structure assignment and NOEs of the main conformation, indicating that the secondary conformation does not involve modification of the β-barrel architecture. Most of the residues producing double peaks cluster in the cis region of strands 1, 2 and 8 (FIG. 20B). Multiple resonance peaks might be explained by close proximity to the flexible N-terminus or by the transient dimeric interactions identified by native mass spectrometry in detergent micelles.

TABLE 6

NMR and refinement statistics for TMB2.3
NMR constraints

| Distance constraints | |
|---|---|
| Total unique NOE | 81 |
| Inter-residue | |
| Sequenial([i - j) = 1) | 28 |
| Medium-range ([i - j) <= 4) | 12 |
| Long-range ([i -j) >= 5) | 41 |
| Interstrand hydrogen bonds * | 72 |
| Total dihedral angle restraints | 204 |
| TALOS Φ | 102 |
| TALOS Ψ | 102 |
| Structure statistics | |
| Violations (mean and s.d)° | |
| NOE constraints (Å) | 0.062 ± 0.001 |
| H-bond constraints (Å) | 0.009 ± 0.002 |
| Dihedral angle constraints (°) | 0.331 ± 0.017 |
| Deviations from idealized geometry | |
| Bond lengths (Å) | 0.001 ± 0.000 |
| Bond angles (°) | 0.388 ± 0.002 |
| impropers (°) | 0.218 ± 0.002 |
| Average pairwise r.m.s. deviation (Å)° | |
| Backbone (β-sheet residues °) | 0.67 ± 0.11 |
| Heavy (β-sheet residues) | 1.63±0.14 |
| Backbone (all residues) | 1.21 ± 0.21 |
| Heavy (all residues) | 2.14 ±0.17 |

a Each H-bond is restrained with two upper limits of 2.5 and 3.5 A for HN-O and N-O, respectively.
b No NOE and H-bond violations are greater than 0.5 Å: no dihedral angle violations are more than 5°.
c Calculated for 20 lowest energy confomers. Ramachandran map analysis. 79.7% most favored, 16.6% allowed, 3.4% generously allowed. 0.3% disallowed.
d. β-sheet residues: 7-17, 21-31, 37-44, 51-58, 66-77, 81-93, 99-106, 113-121

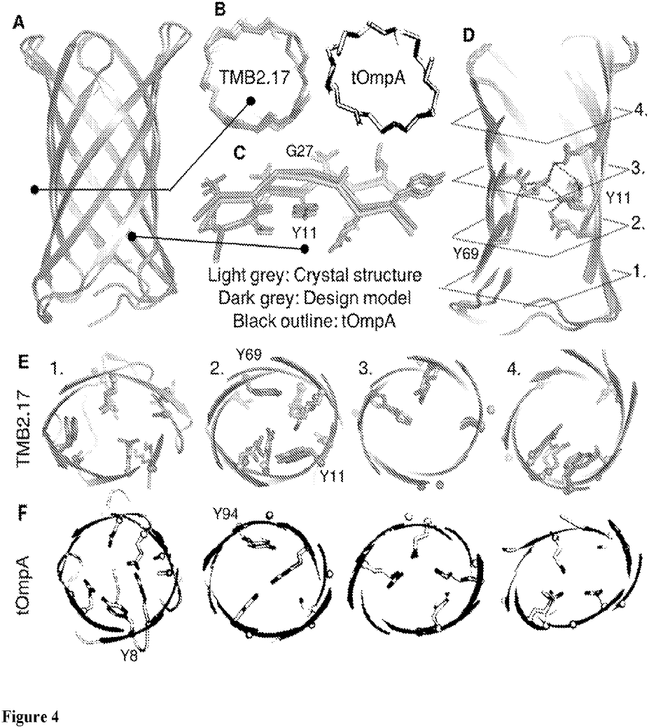

To determine the structure at the atomic level, we crystallized TMB2.17 and solved the structure at 2.05 Å resolution (Table 7). All but two residues located in one trans β-turn were resolved in the electron density map. The crystal structure of TMB2.17 closely matches the design model (1.1 Å backbone RMSD over all residues, FIG. 4D), and the β-barrel has a wide lumen delimited by glycines in an extended conformation that form kinks in the β-strands as designed (FIGS. 4ef). The two YGD/E interactions (Y69, Y11, G27, G89, D39, E103) belonging to the extended mortise/tenon motifs are present in the crystal structure and the second shell of interactions, involving K71, E53 and Q29, is also properly recapitulated with additional interactions to water molecules (FIG. 4g); these extended side-chain hydrogen bond networks fill the lumen of the β-barrel. Overall, the buried amino acid side chain conformations and interactions in the design model are in very good agreement with the crystal structure (FIG. 4h). We compared the core of TMB2.17 to tOmpA, the most similar naturally occurring TMB whose structure has been determined (17% sequence identity, BLAST E-value of 1.6 against the non-redundant database, FIG. 18). The shape of the β-barrel lumen is quite different in the two proteins (FIG. 4E), as are the amino acid identities and packing arrangements of the core sidechains (compare the structure cross sections in FIGS. 4H and 4I).

TABLE 7

Crystallographic Data Collection and Refinement Statistics for TMB2.17 crystal structure
Data collection

| Space group | R3:H |
|---|---|
| Cell dimensions | |
| a, b, c (Å) | 51.08 51.08 116.71 |
| α, β, γ (°) | 90, 90, 120 |
| Resolution (Å) | 41.37 - 2.05 (2.12 - 2.05)° |
| R_{merge} | 0.270(1.375) |
| Rₚᵢᵤₛ | 0.103 (0.519) |
| I/σ(I) | 6.66 (1.18) |
| CC_{½} | 0.995 (0.689) |
| Completeness (%) | 99.54 (98.13) |
| Redundancy | 7.9 (7.7) |
| Refinement | |
| Resolution (A) | 41.37 - 2.05 |
| No. reflections | 7114 (733) |
| R_{work}/R_{free} (%) | 0.260/0.273 (0.292/0.311) |
| No. atoms | 948 |
| Protein | 917 |
| Water | 31 |
| Ramachandran Favored/allowed | 97.41/2.59 |
| Outlier (%) | 00.00 |
| R.m.s deviations | |
| Bond lengths (Å) | 0.003 |
| Bond angles (°) | 0.64 |
| B_{factors} (A¹) | |
| Protein | 36.24 |
| Water | 37.44 |

^{a}Values in parentheses are for the highest-resolution shell.

### Conclusions

The challenge of TMB de novo design is highlighted by the failure of the first three approaches we tried. The sequential approach previously used to build helical transmembrane proteins (6) — design and characterization of soluble proteins and subsequent hydrophobic residue re-surfacing to convert them to membrane proteins — yielded sequences strongly predicted to form amyloid. Designs with more polar cores which had high β-sheet propensity because of the perfect alternation of hydrophobic and polar residues systematically failed to express. Iterative improvement of the design protocol ultimately enabled the generation of a set of sequences with at least 8% of sequences encoding proteins able to adopt a β-barrel fold (based on HSQC NMR). The NMR structure of one of these designs is very close to the design model. The power of our iterative “hypothesize, design, test” approach to explore the sequence landscape of membrane proteins is highlighted by the contrast between the failure in our first rounds of design, and the success in the final round in designing proteins that not only express and fold, but also have atomic structures nearly identical to the design model. The key to this success was introducing glycine kinks, β-bulges and register-defining sidechain interactions and balancing hydrophobicity and β-sheet propensities of the sequences. The extent to which essentially all of the key design features are recapitulated with atomic level accuracy in the crystal structure of TMB2.17 suggests considerable control over TMB structure.

The overall β-sheet propensity and hydrophobicity of our successful designs are in the range of those of naturally-occurring TMBs sequences, suggesting that the natural TMBs might be under a similar negative selection pressure against formation of non-native β-sheet structures in aqueous environment. This is supported by our finding that replacing the tOmpA loops with short strong β-turn-nucleating sequences, but not by suboptimal turn sequences, blocks folding into a native β-barrel structure. Slowing down the folding and assembly of trans hairpins could allow more time for passage of the mostly hydrophilic amino acids in these β-strand connections across the lipid membrane, which likely has a large activation barrier. As well as encoding functional properties, the long loops commonly found on the trans side of the natural TMBs could play a role in slowing folding, although the energetic cost of translocation through the membrane would be much higher, consistent with the different kinetics of folding of tOmpA with long loops and short non-canonical turns. In Gram-negative bacteria, the BAM complex is responsible for accelerating the assembly of natural TMB substrates into the outer membrane by lowering the kinetic barrier to folding. Our design incorporates neither signals for BAM complex association nor evolution-conserved functional motifs and hence represent a “blank slate” for probing the tradeoffs between TMB folding, stability and function, as well as the underlying consequences and evolutionary constraints on OMP trafficking and biogenesis. Finally, the general design principles and methods we have described here — from the definition of the β-barrel architecture to the sequence properties — should be directly applicable to the design of larger pore containing β-barrels. The atomic level of accuracy in sidechain placement demonstrated by the crystal structure of TMB2.17 should enable custom design of transmembrane pores geometric and chemical properties tailored for specific applications.

### REFERENCES AND NOTES

1. R. A. Langan, S. E. Boyken, A. H. Ng, J. A. Samson, G. Dods, A. M. Westbrook, T. H. Nguyen, M. J. Lajoie, Z. Chen. S. Berger, V. K. Mulligan, J. E. Dueber, W. R. P. Novak, H. El-Samad, D. Baker, De novo design of bioactive protein switches. Nature. 572, 205-210 (2019).

2. A. H. Ng, T. H. Nguyen, M. Gómez-Schiavon, G. Dods, R. A. Langan, S. E. Boyken, J. A. Samson, L. M. Waldburger, J. E. Dueber, D. Baker, H. El-Samad, Modular and tunable biological feedback control using a de novo protein switch. Nature. 572, 265-269 (2019).

3. D.-A. Silva, S. Yu, U. Y. Ulge, J. B. Spangler, K. M. Jude, C. Labào-Almeida, L. R. Ali, A. Quijano-Rubio, M. Ruterbusch, I. Leung, T. Biary, S. J. Crowley, E. Marcos, C. D. Walkey, B. D. Weitzner, F. Pardo-Avila, J. Castellanos, L. Carter, L. Stewart, S. R. Riddell, M. Pepper, G. J. L. Bernardes, M. Dougan, K. C. Garcia, D. Baker, De novo design of potent and selective mimics of IL-2 and IL-15. Nature. 565, 186-191 (2019).

4. E. Marcos. T. M. Chidyausiku, A. C. McShan, T. Evangelidis, S. Nerti, L. Carter, L. G. Nivón, A. Davis. G. Oberdorfer, K. Tripsianes, N. G. Sgourakis, D. Baker, De novo design of a non-local β-sheet protein with high stability and accuracy. Nat. Struct. Mol. Biol. 25, 1028-1034 (2018).

5. J. Dou. A. A. Vorobieva, W. Sheffler, L. A. Doyle, H. Park. M. J. Bick, B. Mao, G. W. Foight, M. Y. Lee, L. A. Gagnon, L. Carter, B. Sankaran, S. Ovehinnikov, E. Marcos, P.-S. Huang, J. C. Vaughan, B. L. Stoddard, D. Baker, De novo design of a fluorescence-activating β-barrel. Nature. 561, 485-491 (2018).

6. P. Lu, D. Min, F. DiMaio, K. Y. Wei, M. D. Vahey. S. E. Boyken, Z. Chen, J. A. Fallas, G. Ueda, W. Sheffler, V. K. Mulligan, W. Xu, J. U. Bowie, D. Baker, Accurate computational design of multipass transmembrane proteins. Science. 359, 1042-1046 (2018).

7. N. H. Joh, G. Grigoryan, Y. Wu, W. F. DeGrado, Design of self-assembling transmembrane helical bundles to elucidate principles required for membrane protein folding and ion transport. Philos. Trans. R. Soc. Lond. B Biol. Sci. 372 (2017), doi:10.1098/rstb..2016.0214.

8. J. H. Kleinschmidt, T. den Blaauwen, A. J. Driessen, L. K. Tamm, Outer membrane protein A of Escherichia coli. inserts and folds into lipid bilayers by a concerted mechanism. Biochemistry. 38, 5006-5016 (1999).

9. J. H. Kleinschmidt, L. K. Tamm, Secondary and Tertiary Structure Formation of the β-Barrel Membrane Protein OmpA is Synchronized and Depends on Membrane Thickness. Journal of Molecular Biology. 324 (2002), pp. 319-330.

10. E. J. Danoff, K. G. Fleming, Novel Kinetic Intermediates Populated along the Folding Pathway of the Transmembrane β-Barrel OmpA. Biochemistry. 56, 47-60 (2017).

11. C. P. Moon, S. Kwon, K. G. Fleming, Overcoming hysteresis to attain reversible equilibrium folding for outer membrane phospholipase A in phospholipid bilayers. J. Mol. Biol. 413, 484-494 (2011).

12. D. Chaturvedi. R. Mahalakshmi, Transmembrane β-barrels: Evolution, folding and energetics. Biochim. Biophys. Acta Biomembr. 1859, 2467-2482 (2017).

13. T. Z. Butler, M. Pavienok, I. M. Derrington, M. Niederweis, J. H. Gundlach, Single-molecule DNA detection with an engineered MspA protein nanopore. Proc. Natl. Acad. Sci. U. S. A. 105, 20647-20652 (2008).

14. X. Guan, L.-Q. Gu, S. Cheley, O. Braha, H. Bayley, Stochastic sensing of TNT with a genetically engineered pore. Chembiochem. 6, 1875-1881 (2005).

15. F. Haque, J. Lunn, H. Fang, D. Smithrud, P. Guo, Real-time sensing and discrimination of single chemicals using the channel of phi29 DNA packaging nanomotor. ACS Nano. 6, 3251-3261 (2012).

16. Y.-M. Tu, W. Song, T. Ren, Y.-X. Shen, R. Chowdhury, P. Rajapaksha, T. E. Culp, L. Samineni, C. Lang, A. Thokkadam, D. Carson, Y. Dai, A. Mukthar, M. Zhang, A. Parshin, J. N. Sloand, S. H. Medina, M. Grzelakowski, D. Bhattacharya, W. A. Phillip, E. D. Gomez, R. J. Hickey, Y. Wei, M. Kumar, Rapid fabrication of precise high-throughput filters from membrane protein nanosheets. Nat. Mater. (2020), doi:10.1038/s41563-019-0577-z.

17. T. Surrey, F. Jähnig, Refolding and oriented insertion of a membrane protein into a lipid bilayer. Proc. Nail. Acad. Sci. U. S. A. 89, 7457-7461 (1992).

18. A. D. McLachlan, Gene duplications in the structural evolution of chymotrypsin. J. Mol. Biol. 128, 49-79 (1979).

19. A. G. Murzin, A. M. Lesk, C. Chothia, Principles determining the structure of beta-sheet barrels in proteins. I. A theoretical analysis. J. Mol. Biol. 236, 1369-1381 (1994).

20. M. W. Franklin, J. S. G. Slusky, Tight Turns of Outer Membrane Proteins: An Analysis of Sequence, Structure, and Hydrogen Bonding. J. Mol. Biol. 430, 3251-3265 (2018).

21. N. Koga, R. Tatsumi-Koga, G. Liu, R. Xiao, T. B. Acton, G. T. Montelione, D. Baker, Principles for designing ideal protein structures. Nature. 491, 222-227 (2012).

22. M. A. Lomize, I. D. Pogozheva, H. Joo, H. I. Mosberg, A. L. Lomize, OPM database and PPM web server: resources for positioning of proteins in membranes. Nucleic Acids Res. 40, D370-6 (2012).

23. E. de Alba, E. de Alba, M. Angeles Jiménez, M. Rico, J. L. Nieto, Conformational investigation of designed short linear peptides able to fold into β-hairpin structures in aqueous solution. Folding and Design. 1 (1996), pp. 133-144.

24. T. Blandl, A. G. Cochran, N. J. Skelton, Turn stability in β-hairpin peptides: Investigation of peptides containing 3:5 type I G1 bulge turns. Protein Science. 12 (2003), pp. 237-247.

25. J. S. Richardson, E. D. Getzoff, D. C. Richardson, The beta bulge: a common small unit of nonrepetitive protein structure. Proc. Natl. Acad. Sci. U. S. A. 75, 2574-2578 (1978).

26. W. C. Wimley, Toward genomic identification of β-barrel membrane proteins: Composition and architecture of known structures. Protein Science. 11 (2009), pp. 301-312.

27. L. K. Tamm, H. Hong, B. Liang, Folding and assembly of β-barrel membrane proteins. Biochimica et Biophysica Acta (BBA) - Biomembranes. 1666 (2004), pp. 250-263.

28. J. S. Merkel, L. Regan. Aromatic rescue of glycine in β sheets. Folding and Design. 3 (1998), pp. 449-456.

29. D. L. Leyton, M. D. Johnson, R. Thapa, G. H. M. Huysmans, R. A. Dunstan, N. Celik, H.-H. Shen, D. Loo, M. J. Belousoff, A. W. Purcell, I. R. Henderson, T. Beddoe, J. Rossjohn, L. L. Martin, R. A. Strugnell, T. Lithgow, A mortise-tenon joint in the transmembrane domain modulates autotransporter assembly into bacterial outer membranes. Nat. Commun. 5, 4239 (2014).

30. M. Michalik, M. Orwick-Rydmark, M. Habeck, V. Alva, T. Arnold, D. Linke, An evolutionarily conserved glycine-tyrosine motif forms a folding core in outer membrane proteins. PLoS One. 12, e0182016 (2017).

31. D. P. Ricci, T. J. Silhavy, Outer Membrane Protein insertion by the β-barrel Assembly Machine. EcoSal Plus. 8 (2019), doi:10.1128/ecosalplus.ESP-0035-2018.

32. M. Fioroni, T. Dworeck, F. Rodriguez-Ropero, β-barrel Channel Proteins as Tools in Nanotechnology: Biology, Basic Science and Advanced Applications (Springer Science & Business Media, 2013).

33. R. D. Requião, L. Fernandes, H. J. A. de Souza, S. Rossetto, T. Domitrovic, F. L. Palhano, Protein charge distribution in proteomes and its impact on translation. PLoS Comput. Biol. 13, e1005549 (2017).

34. E. J. Danoff, K. G. Fleming, Aqueous, Unfolded OmpA Forms Amyloid-Like Fibrils upon Self-Association. PLoS One. 10, e0132301 (2015).

35. N. Noinaj, A. J. Kuszak, S. K. Buchanan, Heat Modifiability of Outer Membrane Proteins from Gram-Negative Bacteria. Methods Mol. Biol. 1329, 51-56 (2015).

36. P.-Y. Chen, C.-K. Lin, C.-T. Lee, H. Jan, S. I. Chan, Effects of turn residues in directing the formation of the β-sheet and in the stability of the β-sheet. Protein Science. 10 (2001), pp. 1794-1800.

37. R. Koebnik. Structural and Functional Roles of the Surface-Exposed Loops of the β-Barrel Membrane Protein OmpA fromEscherichia coli. . Journal of Bacteriology. 181 (1999), pp. 3688-3694.

38. E. J. Danoff, K. G. Fleming, The soluble, periplasmic domain of OmpA folds as an independent unit and displays chaperone activity by reducing the self-association propensity of the unfolded OmpA transmembrane β-barrel. Biophys. Chem. 159, 194-204 (2011).

39. S. E. Boyken, Z. Chen, B. Groves, R. A. Langan, G. Oberdorfer, A. Ford, J. M. Gilmore, C. Xu, F. DiMaio, J. H. Pereira, B. Sankaran, G. Seelig, P. H. Zwart, D. Baker, De novo design of protein homo-oligomers with modular hydrogen-bond network-mediated specificity. Science. 352, 680-687 (2016).

40. D. L. Minor, P. S. Kim, Measurement of the β-sheet-forming propensities of amino acids. Nature. 367 (1994), pp. 660-663.

41. J. A. Stapleton, T. A. Whitehead, V. Nanda, Computational redesign of the lipid-facing surface of the outer membrane protein OmpA. Proc. Natl. Acad. Sci. U. S. A. 112, 9632-9637 (2015).

42. T. Kortemme, A. V. Morozov, D. Baker, An Orientation-dependent Hydrogen Bonding Potential Improves Prediction of Specificity and Structure for Proteins and Protein-Protein Complexes. Journal of Molecular Biology. 326 (2003), pp. 1239-1259.

43. A. Ebie Tan, N. K. Burgess, D. S. DeAndrade, J. D. Marold, K. G. Fleming, Self-association of unfolded outer membrane proteins. Macromol. Biosci. 10, 763-767 (2010).

44. J.-L. Popot, Folding membrane proteins in vitro: a table and some comments. Arch. Biochem. Biophys. 564, 314-326 (2014).

45. A. Schüßler, S. Herwig, J. H. Kleinschmidt, Kinetics of Insertion and Folding of Outer Membrane Proteins by Gel Electrophoresis. Methods Mol. Biol. 2003, 145-162 (2019).

46. H. Hong, L. K. Tamm, Elastic coupling of integral membrane protein stability to lipid bilayer forces. Proceedings of the National Academy of Sciences. 101 (2004), pp. 4065-4070.

47. G. H. M. Huysmans, S. A. Baldwin, D. J. Brockwell, S. E. Radford, The transition state for folding of an outer membrane protein. Proc. Natl. Acad. Sci. U. S. A. 107, 4099-4104 (2010).

48. C. L. Pocanschi, G. J. Patel, D. Marsh, J. H. Kleinschmidt, Curvature elasticity and refolding of OmpA in large unilamellar vesicles. Biophys. J. 91, L75-7 (2006).

49. S. Ohnishi, K. Kameyama, Escherichia coli. OmpA retains a folded structure in the presence of sodium dodecyl sulfate due to a high kinetic barrier to unfolding. Biochim. Biophys. Acta. 1515, 159-166 (2001).

50. J. H. Kleinschmidt, L. K. Tamm, Folding Intermediates of a β-Barrel Membrane Protein. Kinetic Evidence for a Multi-Step Membrane Insertion Mechanism†,‡. Biochemistry. 35 (1996), pp. 12993-13000.

51. N. K. Burgess, T. P. Dao, A. M. Stanley, K. G. Fleming, Beta-barrel proteins that reside in the Escherichia coli. outer membrane in vivo demonstrate varied folding behavior in vitro. J. Biol. Chem. 283, 26748-26758 (2008).

52. Y. Shen, F. Delaglio, G. Cornilescu, A. Bax, TALOS : a hybrid method for predicting protein backbone torsion angles from NMR chemical shifts. Journal of Biomolecular NMR. 44 (2009), pp. 213-223.

53. J. M. Hemmingsen, K. M. Gernert, J. S. Richardson, D. C. Richardson, The tyrosine corner: A feature of most greek key β-barrel proteins. Protein Science. 3 (1994), pp. 1927-1937.

54. C. M. Bishop, W. F. Walkenhorst, W. C. Wimley, Folding of β-sheets in membranes: specificity and promiscuity in peptide model systems. Journal of Molecular Biology. 309 (2001), pp. 975-988.

55. A. Perez-Rathke. M. A. Fahie, C. Chisholm, J. Liang, M. Chen, Mechanism of OmpG pH-Dependent Gating from Loop Ensemble and Single Channel Studies. J. Am. Chem. Soc. 140, 1105-1115 (2018).

56. J. Vogt, G. E. Schulz, The structure of the outer membrane protein OmpX from Escherichia coli. reveals possible mechanisms of virulence. Structure. 7 (1999), pp. 1301-1309.

57. F. Endriss, V. Braun. Loop deletions indicate regions important for FhuA transport and receptor functions in Escherichia coli. J. Bacteriol. 186, 4818-4823 (2004).

58. 1. Kucharska, P. Seelheim, T. Edrington, B. Liang, L. K. Tamm, OprG Harnesses the Dynamics of its Extracellular Loops to Transport Small Amino Acids across the Outer Membrane of Pseudomonas aeruginosa. Structure. 23, 2234-2245 (2015).

59. C. P. Moon, N. R. Zaccai, P. J. Fleming, D. Gessmann, K. G. Fleming, Membrane protein thermodynamic stability may serve as the energy sink for sorting in the periplasm. Proc. Natl. Acad. Sci. U. S. A. 110, 4285-4290 (2013).

60. C. P. Moon, K. G. Fleming, Side-chain hydrophobicity scale derived from transmembrane protein folding into lipid bilayers. Proc. Natl. Acad. Sci. U.S.A. 108, 10174-10177 (2011).

61. H. Hong, S. Park, R. H. F. Jiménez, D. Rinehart, L. K. Tamm, Role of aromatic side chains in the folding and thermodynamic stability of integral membrane proteins. J. Am. Chem. Soc. 129, 8320-8327 (2007).

62. M. Källberg, G. Margaryan, S. Wang, J. Ma, J. Xu, RaptorX server: A Resource for Template-Based Protein Structure Modeling. Methods in Molecular Biology (2014), pp. 17-27.

63. J. Kyte, R. F. Doolittle, A simple method for displaying the hydropathic character of a protein. J. Mol. Biol. 157. 105-132 (1982).

64. A.-M. Femandez-Escamilla, F. Rousseau, J. Schymkowitz, L. Serrano, Prediction of sequence-dependent and mutational effects on the aggregation of peptides and proteins. Nat. Biotechnol. 22, 1302-1306 (2004).

65. A. Stein, T. Kortemme, Improvements to robotics-inspired conformational sampling in rosetta. PLoS One. 8, c63090 (2013).

### Supplementary Materials

### Material and Methods

Computational de novo design of a new protein with the Rosetta® molecular modelling suite has two steps: first, a protein backbone is built, which is then used to guide the search for low energy sequence/structure pairs.

### Backbone Generation

The same backbone generation approach (“backbone_generation.xml”) was applied throughout this study and was described elsewhere (5, 66). The desired protein backbone was described in a blueprint format (“TMB_blueprint”), where every residue in the protein was assigned a secondary structure type and a Ramachandran plot bin using Rosetta® ABEGO type (67). The backbone-to-backbone hydrogen bond interactions for the protein were specified with constraints (“hbond_constraints”). To achieve control over the type of β-turns and torsional irregularities incorporated into the designed backbones, specific Ramachandran bins and hydrogen bonding patterns were assigned to β-turn, β-bulge and glycine kink residues. To design type I β-turns (3:5) on the trans side of the β-barrel, the ABEGO sequence “AAG” was used while type I β-turns on the cis side were designed with the ABEGO sequence “AA”. A β-bulge was defined as a single residue in the alpha region of the ramachandran plot (“A” ABEGO type) with β-strand secondary structure. A glycine kink was defined as a single residue with a positive φ backbone angle (“E” ABEGO type) and a β-strand secondary structure. The rationale to design blueprints and specify constraints specific to β-barrels is provided in the supplementary text. The blueprint and constraints are used as input to the BluePrintBDR application (21) in Rosetta® (“backbone_generation.xml”), which uses the information in the blueprint to pick fragments (9-mers and 3-mers) from crystal structures in the PDB and uses these fragments to search the structure space for low-energy structures using a Monte Carlo algorithm. Achieving enough conformational sampling to build all the hydrogen bonds in the β-barrel is computationally challenging, so the models produced by the BluePrintBDR are further minimized in the presence of the constraints and Rosetta® hydrogen bond potential (hbond_lr_bb) to drive the pairing between the β-strands. Every hydrogen bond is described with a distance constraint (between the N and O backbone atoms) and an angle constraint (the N-H-O angle). Such detailed description of the geometry of the interactions is necessary to compensate for Rosetta® inability to detect and score the hydrogen bonds that are located more than 5 Å apart in the input model and that are therefore excluded from the calculations of the interaction graph. The minimization step is done using a generalized rama potential (“Rama_XPG_3level.txt”) and a coarse-grained energy function (Rosetta® centroid energy function), that was specifically optimized to balance long-range hydrogen bonding requirements with the local torsion angle requirements (“fldsgn_cen_omega02.wts”). The output of this design protocol is a set of three-dimensional protein backbone models with valine residues as placeholders at every position, except at the predefined glycine kink positions. High quality backbones to use in the sequence design step were selected based on the vdw, rama and omega scoring terms (“backbones_analysis.ipynb”). In this study. 10,000 backbone generation trajectories were necessary to obtain 200 backbones satisfying the quality criteria.

### Combinatorial Sequence Redesign of Surface Residues of Water Soluble Β-Barrels

The PDB coordinates of the previously designed water soluble beta-barrels (5) were used as template to redesign (“design_surface.xml”) polar surface-exposed positions to hydrophobic amino acids (VILAF, resfile in “all.resfile”), with additional constraints (“girdle_cst”) enforcing specific rotamers for aromatic girdle residues at the water/lipid interface. The ref2015 default Rosetta® energy function (68) with modified reference energy for phenylalanine was used to limit the density of phenylalanines designed on the hydrophobic surface and match the distributions observed in naturally occurring TMBs (ref2015_F.wts). The lowest energy design was selected for each starting crystal structure out of five independent design trajectories.

### Combinatorial Sequence Design

For all three generations of designs reported in this study (TMB0, TMB1, and TMB2), the search for a low-energy sequence was done over several rounds of iterative design following a genetic algorithm approach (~10% best scoring designs from one round of design were used as input for the next round of design). If necessary, changes were implemented to obtain designs to more closely match the hypothetical model that was tested.

### Combinatorial Sequence Design of the Design Set TMB0

The set TMB0 was designed over four rounds of combinatorial sequence design (“design_gly.xml”). For all rounds of design, only polar amino acids were allowed in the core of the β-barrel, with the exception of the two tyrosines that occur in the mortise/tenon motifs; hydrophobic amino acids were allowed on the surface and aromatic amino acids at the lipid/water boundaries. All allowed amino acid combinations were specified in a resfile (“resfile”). After each round, the designs were selected based on the following criteria: 1) the correct rotameric state of the tyrosines 10 and 68, belonging to the mortise/tenon motifs, which is enforced with constraints during design (“mortise_tenon_est”), and 2) the Rosetta® total_score and four backbone quality metrics omega, rama_prepro, p_aa_pp, and hbond_lr_bb. The designs that scored better than the average for all four of the Rosetta® metrics were selected for the next round of design (“analysis_21_02_16.ipynb”). These criteria typically eliminated approximately 90% of the initial designs with a correct mortise/tenon motif. For the last (fourth) round of design, a modified energy function with increased weights on the electrostatic interactions was used (“ref2015_fa_elec.wts”) to favor more charged residues in the core. We hypothesized that a sharper contrast in hydrophobicity between the core and the surface of the β-barrel could improve the typical hydrophobic/polar alternation of residues characteristic of β-strands and hence improve β-strand secondary structure definition. Good definition of secondary structure elements on the sequence level is one of the key criteria for success of the design of new water-soluble protein folds (21).

### Combinatorial Sequence Design of the Design Set TMB1

To generate the set of designs TMB1, a small subset of designs generated after the third iteration in TMB0 (before the increase of the fa_elec weight to design more charged residues in the core) were selected. The surface was designed one more time with hydrophobic residues (“design.xml”, “surface.resfile”) to more closely match the amino acid probabilities on the surface of naturally occuring TMBs (“surface.comp”).

### Combinatorial Sequence Design of the Design Set TMB2

The first round of sequence design of the set TMB2 consisted of two stages. First, the centroid models from the backbone generation step were pre-designed in full-atom mode with Rosetta® default energy function ref2015 (68) (“design_1.xml”) and by specifying allowed amino acids in the core and surface based on the inside-out model (“resfile_I”). The tyrosines in the mortise/tenon motifs were included at this stage and the specific rotamers characteristic of these interactions were enforced with constraints (“constraints_1”). The designs that scored better than average for Rosetta® total_score, omega, rama_prepro and hbond_lr_bb scores (“backbones_analysis.ipynb”) were selected to serve as input models for the next design stage.

In the second stage, we searched for all the possible positions of aspartate or glutamate side-chains to act as a hydrogen bond acceptor to the tyrosines in the two mortise/tenon motifs. All the residues in the designs, except glycines, prolines and the two tyrosines (that belong to the mortise/tenon motifs), were mutated to alanine and the models were exhaustively searched for possible polar interactions stemming from the found D/E using the Rosetta® HBNet protocol (39) (“hbnet.xml”). The parameters of the HBNet protocol, hb_threshold in particular, were adjusted to be able to consistently recover hydrogen bond interactions to the extent found in that of relaxed crystal structures of native TMBs. Each output model from the “hbnet.xml” run was relaxed with coordinate constraints (“fast_relax.xml”). The HBNet solutions found for each tyrosine of the mortise/tenon motif were recombined to generate all possible combinations of one or two designed mortise/tenon motifs or YGD/E motifs for every input backbone (“get_all_motifs.py”).

The models generated with the “get_all_motifs.py” script (poly-alanine with the glycines, prolines and the designed YGD/E motifs) were used as input for the next round of sequence design. Three additional rounds of combinatorial sequence design were performed. The core and surface positions were designed independently in each round of design.

For each input model, a constraints file and a resfile were generated. The resfile defines the allowed amino acids in the β-turn regions and amino acid identities of the residues in the designed YGD/E motifs. A constraints file was generated for each model to enforce the rotameric state of the tyrosine(s) in the motif(s) and to maintain the hydrogen bond interaction to the negatively charged amino acid. The resfile and constraints files were generated with the “get_all_motifs.py” script. The best designs were selected based on the energy of the hydrogen bond interactions between the tyrosine(s) and the negatively charged residue(s) and based on the total energy per residue of these negatively charged residue(s) evaluated with Rosetta® (“select_best_motif_round2.ipynb”).

In the surface design stage of round two (“design_surf_round2.xml”), the aromatic residues forming the aromatic girdle at the water/lipid boundaries were introduced (“surface_round2.resfile”) and their rotameric state enforced with constraints (“constraints_surface_round2”) Since the core residues were allowed to repack during the surface residues design stage, the designs that retained low-energy YGD/E motifs were selected to move onto round three (“select_best_motif_round2.ipynb”).

All the designs from the core design stage of round three as well as the designs selected after round two were collected and the properties of the core polar interactions networks were analyzed in more detail. A custom Rosetta® XML script (“filters.xml”) was run to score the models based on packing of side chains around the glycine kinks, the packing of side-chains around the core polar network residues, and the number of unsatisfied hydrogen bonds in the core network of polar residues

A Rosetta® HBNet protocol was used to identify the existing hydrogen bond networks in the core of each design.

The outputs of the two scripts were used to compute the size, energy and saturation of the networks and the number of satisfied and unsatisfied hydrogen bonds. These metrics, Rosetta® side-chain hydrogen bond score (hbond_sc) and the metrics computed using the “filters.xml” script were used to select the designs with the most extensive and stable core networks for the next round of surface design (“filter_networks.ipynb”).

For the surface design stage of round three (“design_surf_round3.xml”), glycines were allowed in lipid-exposed surface positions (“surface_gly_round3.resfile”) and the weight on the long-range hydrogen bond potential (hbond_lr_bb) was increased to 2.0 to find strained positions on surface and design them into glycine. The rotameric state of the residues belonging to the aromatic girdles was enforced with constraints. The core networks were allowed to repack during the surface design stage, and the designs with the highest retention of these networks after repacking and lowest Rosetta™ omega score were selected (“analyse_round3_surf.ipynb”). Seven hundred and seventy-five designs were selected following this procedure. After manual inspection of the core network of hydrogen bonds, two hundred and four designs were excluded for presenting unsatisfied polar atoms potentially buried in hydrophobic pockets (which is difficult to detect automatically in a reliable way). The four hundred and eighty-eight designs with the lowest total side-chain to side-chain hydrogen bond energy (hbond_sc) were selected for the last stage of combinatorial sequence design (“cluster_round4.ipynb”). The designs were manually clustered based on the similarity of their core hydrogen bond networks (“cluster_round4.ipynb”). The amino acids on the surface of these designs were designed one more time (“design_surf_round4.xml”) to incorporate phenylalanines and therefore increase the hydrophobicity of the lipid-exposed surface of the β-barrel. Since it is an artefact of Rosetta® energy function to excessively favor phenylalanine amino acids, the reference weight for phenylalanines was modified in the default energy function (“ref2015_F4.wts”) to incorporate phenylalanines at a rate similar to what is observed in naturally occuring TMBs. The rotameric state of the residues belonging to the aromatic girdle was enforced with constraints that were used for the previous rounds of surface design. A resfile was used to define allowed amino acids on the lipid exposed surface (VILAF) excluding the positions that have been previously designed as glycine or proline. For each input model, ten independent surface design trajectories were run and the lowest energy design (total_score) was selected (“analyse_clusters.ipynb”).

The ninety ordered designs were selected to span each of these structural clusters as well as a broad range of hydrophobicity of the core and propensity for β-sheet and alpha-helix secondary structure (as predicted with RaptorX®). The analysis and selection criteria can be found in the provided Jupyter® Notebooks (“analyse_round4.ipynb” to select TMB2.1 to TMB2.20 that have unique core networks that do not belong to any existing cluster; “analyse_clusters.ipynb” to select designs TMB2.21 to TMB2.90 from the network clusters). The placeholder sequences of the trans β-turn used throughout the design process were replaced with the suboptimal sequences necessary for TMB folding identified in this study.

### Rosetta®™ Simulations With PPM Predictions

The protein backbones for the tested topologies were generated based on blueprints and constraints files provided in the GitHub® repository. A sequence was designed for each of the 20-25 best scoring backbones following the inside-out model and with aromatic residues at membrane anchoring positions to the β-turns to define the aromatic girdle. The 20-25 models were submitted to the PPM server to define its position in the lipid bilayer. The tilt angles, water-to-lipid partition energies and hydrophobic thicknesses were averaged per topology. For every tested topology, an average molecular model was generated by averaging the heavy atoms of the proteins as well as the planes defining the lipid membrane leaflets (“average_hydrophobic_thickness.ipynb”). Such an average model was used to verify the continuity of the hydrophobic thickness.

### Computational Simulation of the Structure/Energy Landscape of Β-Turns

To compute structure/energy landscapes for the β-turn sequences, one low energy poly-valine TMB backbone was selected for the simulation and the trans β-turn positions and two additional β-strand flanking residues on both sides of the β-turn were mutated to the target sequence. The backbones conformations were readjusted to the new sequences by running the Rosetta® FastRelax protocol. Two hundred fifty loop conformations were generated by independent KIC sampling and scored with Rosetta’s default energy function. To do so the Rosetta® loopmodel protocol was run with KIC backbone perturbation.

The RMSD of each generated loop conformation to the conformation in the starting model (canonical backbone for the 3:5 type I β-turn with a GI β-bulge) was calculated.

### Amino Acid Propensities in Naturally Occuring 8-Strands TMBs

The multiple sequence alignments (MSA) were generated by searching for homologs of 8-strands TMBs with crystal structures deposited in the PDB (1qjp, 2flv, 1thq, 1qj8, 2k01, 2mlh, 1p4t, 4fav, 4rlc, 2n61, 2lhf, 2erv, 3qra) using GREMLIN (69). The sequences in the MSA were merged and filtered for maximum 90% sequence similarity with CD-HIT (70). The MSA is provided in the GitHub® repository.

To compute the amino acid compositions of the transmembrane β-strands and the β-turns, we assumed that the interaction with the lipid membrane constrains the evolution of the β-barrel architecture and results in constant position of the transmembrane regions in the sequence of the protein. This hypothesis was supported by the comparison between the amino acid compositions computed with our method and the statistic reported in a previous study based on crystal structures of TMBs with different strand lengths (71) (FIGS. 7). The regions of the MSA corresponding to the transmembrane β-strands or the β-turns were identified based on the crystal structures of the query sequences, extracted from the MSA and used for the downstream analysis. The transmembrane β-strand regions were defined as the span from the membrane anchor position from one side of the membrane to the membrane anchor position to the other side of the membrane.

To investigate how the well the β-turn structure is defined by the sequence profiles derived from the MSA, we used Rosetta® fragment_picker protocol (72) to pick fragments from crystal structures in the PDB . Only the sequence profiles from the MSA were considered for fragment picking. We compared the cis and trans β-turn sequence profiles for identical types of β-turn backbones on the same protein to avoid potential bias from MSA depth.

### Protein Expression and Purification

Codon-optimized genes encoding the TMB and tOmpA loop variants were synthesized and cloned into the pET-29 vector (Integrated DNA technologies). The natural tOmpA and full-length OmpA genes were cloned into the same vector from the E. coli K-12 strain. The OmpA, tOmpA and OmpAAG constructs were originally expressed with a C-terminal 6×His-tag fusion, which did not influence the ability of the protein to fold into lipid membrane or detergent micelles. However, the OmpTrans and TMB designs were not fused to the 6×His-tag because his-tagged proteins were found to produce less compact and more difficult to purify inclusion bodies. Plasmids were transformed into BL21*(DE3) E. coli strain (NEB). Protein expression was induced by overnight growth at 37° C. in the Studier autoinduction medium and replicated at least twice for the designs from set TMB0, the designs TMB2.1 to TMB2.20 and the designs TMB2.21-TMB2.90 that failed to express. To isolate the proteins in inclusion bodies, the cells were lysed either by sonication (50 ml cultures for design screening) or with a MicroFluidizer® (Microfluidics) in lysis buffer (50 mM Tris pH 8.0, 40 mM EDTA pH 8.0). The cell lysate was incubated for 60 min at 4° C. with 0.1 % of Brij-35. The inclusion bodies were collected by centrifugation, re-suspended in the washing buffer (10 mM Tris pH 8.0, 1 mM EDTA pH 8.0) by sonication and pelleted again. The washing step was repeated three times. The pellets were stored at -20° C. The proteins prepared for the small scale screening assay were dissolved in 6 M urea and used immediately. The proteins prepared for biochemical and structural characterization were first dissolved in 8 M guanidinium chloride (GuCl) and further purified by Akta® Pure fast protein liquid chromatography (GE Healthcare) using a Superdex® 75 increase 10/300 GL column (GE Healthcare) in denaturing conditions.

### Expression of ¹⁵N and ¹H-¹⁵N-¹³C Isatopically Labelled Proteins

### ¹⁵N Isotopically Labelled Proteins

A LB media starter culture was prepared at equal volume to the desired expression volume and grown overnight at 37° C., 200 rpm. Cells were harvested at 4,000 RPM, 4° C. for 10 minutes or until a solid pellet forms. Cell pellet was gently resuspended (do not vortex) with M9 minimal media (30 mM Na₂HPO₄, 20 mM KH₂PO₄, 10 mM NaCl, 10 mM NH₄Cl, 0.2% glucose, 1 mM MgSO₄, 0.1 mM CaCl₂, 0.01 g/L biotin, 0.01 g/L thiarnin, 1× trace metals, appropriate antibiotic) with ¹⁵N-NH₄Cl (Cambridge Isotopes). Cultures were grown at 37° C., 200 rpm. OD₆₀₀ was measured after 2 hours after inoculation. Cultures were induced with 0.5 mM IPTG at OD₆₀₀0.8-1.0 and grown overnight at 22° C., 200 rpm. 500µL of pre-induced culture was retained for later analysis. Cells were harvested at 4,000 RPM, 4° C. for 10 minutes. Supernatant was discarded and the cell pellet was stored at -80° C. or used immediately for protein purification. Protein expression was assessed via SDS-PAGE with pre- and post-induction retain samples.

### ¹H-¹⁵N-¹³C Isotopically Labelled Proteins

Due to decreased cell growth and protein expression yields in the presence of D₂O, the gradual introduction of deuterated media is recommended. A 5 mL starter culture in 100% H₂O LB media was prepared and the percentage of D₂O LB media was increased in a stepwise fashion (100% H₂O:0% D₂O, 75:25, 50:50, 25:75, 0:100). Cultures were grown at 37° C., 200 rpm overnight prior to a 1:10 inoculation ratio for subsequent steps. 0.2% glucose was added to LB media to promote cell growth. A glycerol stock was prepared when the bacterial culture has adopted 100% deuterated media, the remaining overnight was used to start an expression culture. Protein was expressed and harvested using the previously described ¹⁵N isotopically labelled proteins protocol using M9 media containing ¹⁵N-NH₄Cl (Cambridge Isotopes) and 0.2% ¹³C-glucose (Cambridge Isotopes), in deuterium.

### Screening Assay in Deterpent Micelles

The first twenty TMB2 designs (and their variants with tOmpA loop inserts) were tested in DDM detergent micelles. We later switched to DPC detergent for improved refolding efficiency (by comparing the refolding efficiency of a few designs in both detergents by HSQC NMR) and to simplify the interpretation of the results. For a few designs, the screening assay was repeated in OG detergent micelles. Before the folding experiment, the protein pellets were dissolved in urea and centrifuged 30 min at maximum speed. The concentration of protein in the supernatant was measured using a nanodrop and the stocks were diluted to 80 µM. 250 µM of the 80 µM stock solutions were diluted drop-by-drop into 5 ml of vortexed refolding buffer (20 mM Tris pH 8.0, 150 mM NaCl, 2X CMC detergent). DPC detergent was used at a concentration of 0.1%; DDM detergent was used at a concentration of 0.02%; OG detergent was used at a concentration of 1%. In parallel, 250 µM of the 80 µM stock solutions were diluted drop-by-drop into 5 ml of TBS buffer (20 mM Tris pH 8.0, 150 mM NaCl) to test the solubility of the design in the absence of detergent. The samples were incubated overnight at 4° C. on a rocker. To assess protein solubility, 20 µl of each sample and the corresponding control without detergent were centrifuged 30 min at maximum speed and analyzed on SDS-PAGE. A non-centrifuged sample was analyzed alongside them to provide the total protein band. The samples prepared in detergent were concentrated to 1ml in an Amicon Ultracentrifugation device with a cut-off of 10 kDa (Merck Millipore). After centrifugation for 30 min at maximum speed, the protein/detergent complexes were separated from larger aggregates using a Superdex® 200 increase 10/300 GL SEC column (GE Healthcare) in the refolding buffer. If a major species with a retention volume compatible with a monomeric 8-strands TMB was detected by SEC, that species was further tested for the presence of a heat-modifiable species (SDS-PAGE band-shift assay), for resistance to proteases and for a β-sheet characteristic far-UV CD spectrum.

### Far UV Circular Dichroism Spectroscopy

For the TMB screening in detergent micelles, the protein/detergent complex collected out of SEC was directly analyzed by CD spectrometry in SEC buffer (20 mM Tris pH 8.0, 150 mM NaCl, 2X CMC detergent). CD spectra were obtained using a Jasco model J-1500 spectropolarimeter over a wavelength range of 260-190 nm. The temperature was controlled with a Peltier and spectra were recorded every 10° C., from 25° C. to 95° C. One last spectrum was recorded after cooling the sample down back to 25° C. For detailed biophysical characterization of designs T-MB2.3 and TMB2.17 in synthetic lipid membranes, the TMBs denatured in 50 mM glycine-NaOH pH 9.5, 8 M urea were diluted into DUPC LUVs in 50 mM glycine-NaOH pH 9.5 containing 0.24 M, 2 M and 8 M urea, and folding was allowed to proceed overnight at 25° C. The final protein concentration was 6 µM the lipid/protein ratio (LPR) was 600:1 (mol/mol). Average CD spectra from four repeats were obtained using a Chirascan® Plus (Applied Photophysics) spectropolarimeter equipped with Peltier temperature controller set at 25° C., over a wavelength range of 260-190 nm, a digital integration time of 2 seconds, and a 2 nm bandwidth.

### Protease Challenge

Trypsin-EDTA (0.25%) solution was purchased from Life Technologies and stored at stock concentration (2.5 mg/mL) at -20° C. α-Chymotrypsin from bovine pancreas was purchased from Sigma-Aldrich as lyophilized powder and stored at 1 mg/mL in TBS +100 mM CaCl₂: at -20° C. A sample of the protein/detergent complex collected out of SEC was directly subject to a test for protease resistance. 19 µl of the protein/detergent sample were mixed with 1 µl of DTA and another 19 µl sample was treated with 1 µl of α-Chymotrypsin. The samples were incubated 15 min at Room Temperature. The reaction was quenched with 2X Laemmli Sample Buffer (BioRad). The samples were heated at 95° C. for 10 min and analyzed on SDS-PAGE gel (Any kD® Mini-PROTEANⓇ TGX® Precast Protein Gels, BioRad) alongside an undigested sample.

### SDS-PAGE Band-Shift Assay

In the context of TMB screening in detergent micelles, 2× 20 µl of each sample collected from SEC were mixed with 2X Laemmli Sample Buffer (BioRad). For each tested protein, one sample was heated at 95° C. for 10 min while the other sample was kept at room temperature. The samples were analyzed on a SDS-PAGE gel (Any kD® Mini-PROTEAN®: TGX® Precast Protein Gels, BioRad). For detailed biophysical characterization of designs TMB2.3 and 1.M.B2.17, samples of the folding reaction used for far-UV CD were mixed with 4× SDS-PAGE loading buffer (200 mM Tris-HCI pH 6.8, 6% (w/v) SDS, 40%, (v/v) glycerol, 0.004% (w/v) bromophenol blue, and folded/unfolded species were resolved on a 15% (w/v) acrylamide/bis-acrylamide (37.5:1) Tris-Tricine SDS-PAGE gel at pH 8.45 operating at 60 mA for 90 minutes at room temperature. Boiled samples were heated to >95° C. for 10 minutes. Gels were stained with InstantBlue® (Expedeon) and imaged using an Alliance Q9 Advanced gel doc (UVITEC, Cambridge, UK).

### Equilibrium Folding/Unfolding by Tryptophan Fluorescence

To determine the urea dependence of TMB folding, urea denatured TMBs in 50 mM glycine-NaOH pH 9.5, 8 M urea were diluted into DUPC LUVs at an LPR of 600:1 (mol/mol) in 50 mM glycine-NaOH pH 9.5 containing 0.24-9 M urea, and folding was allowed to proceed overnight at 25° C. To measure the urea dependence of unfolding, TMBs were initially folded in DUPC LUVs at an LPR of 600:1 (mol/mol) in 50 mM glycine-NaOH pH 9.5, 2 M urea overnight at 25° C. The folded TMB stock was then diluted 10-fold into 50 mM glycine-NaOH pH 9.5 containing 2-9 M urea and incubated overnight at 25° C. to initiate unfolding. The final protein concentration was 0.4 µM and the LPR was 600:1 (mol/mol). Tryptophan fluorescence emission spectra were obtained using a PT1 QuantaMaster® spectrofluorometer (Photon Technology International) in QS quartz cuvettes with excitation slits set to 1 nm and emission slits set to 5 run. Fluorescence was excited at 280 nm and emission spectra were acquired between 300-400 nm using a step size of 1 nm and an integration time 1 second. The fluorescence intensity at 335 nm was plotted against the urea concentration and data were fitted with a sigmoid function to extract the urea concentration midpoint for folding (Cm^{f}) and unfolding (Cm^{UF}).

### Folding Kinetics Measured Using Tryptophan Fluorescence

Kinetics of TMB folding into DUPC and DMPC LUVs were measured at a final OMP concentration of 0.4 µM and an LPR of 3200:1(mol/mol). The TMB unfolded proteins were diluted 20-fold from 8 M urea into LUVs created from DUPC or DMPC in 50 mM glycine-NaOH pH 9.5 containing 2 M or 9 M urea. The choice of using 2 M urea to monitor TMB folding was made based on the results of the band-shift assay on SDS-PAGE (FIG. 17), that showed partial aggregation of tOmpA at lower concentrations of urea. TMBs were also diluted from 8 M urea in 2 M urea without lipids to determine the lipid dependence of folding. Upon addition of denatured TMBs to LUVs in the folding buffer pre-equilibrated at 25° C., the reaction was mixed rapidly and fluorescence emission was monitored at 335 nm following excitation at 280 nm over 30 minutes. Excitation slits were set to 0.5 nm, emission slits were 5 nm, the bandwidth was 1 nm and integration time was 2 seconds. Kinetics were measured in triplicate and, where possible, were globally fitted to a single exponential function to extract folding rate constants.

### NMR Spectroscopy and Structural Calculations

All NMR spectra were collected on a Bruker Avance® 800 MHz spectrometer equipped with a cold-probe. For initial sample optimization and screening, 2D TROSY-HSQC spectra were collected for ¹⁵N-labeled samples. For backbone assignments of the TMB2.3, TROSY-versions of 3D experiments [HNCA, HN(CA)CB, HNCO, HN(CA)CO] were collect on a ²H, ¹³C, ¹⁵N-labeled sample with a non-uniformed sampling (NUS) technique. Two 3D NOE experiments, ¹⁵N-¹⁴N-¹H HSQC-NOESY-HSQC and ¹⁵N-¹H-¹H NOESY-TROSY, were performed with mixing times of 120 ms, also in the NUS mode. In addition, a TROSY-based 2D ¹H-¹⁵N heteronuclear NOE experiment was collected with a saturation recovery delay of 5 s with an interleaved approach. All spectra were processed and analyzed with NMRPipe (73) and Sparky (74), and in particular, NUS scheduling and reconstruction were carried out with hmsIST (73).

The presence of a well-ordered TMB2.3 structure was supported by NMR dynamics measurements. We measured ¹H-¹⁵N heteronuclear NOE values for non-overlapped 101 residues, the high average of 0.83 ± 0.11 indicated restricted motions for the whole protein. Dihedral angle restraints were predicted from the TALOS-N® program (76) on the basis of the experimental Ca, Cb, CO, N, and HN chemical shifts. Good predictions from TALOS-N were converted to input values for structural calculations with tolerances at either twice the standard deviations or 20°, whichever the larger value. All assigned NOE peaks were converted to NOE distances using their peak height values and calibrated based on the fact that the average HN-UN distance between anti-parallel beta-strands is 3.3 Å. For structure calculations, the distances were categorized as having strong, medium, and weak NOEs with upper limits of 3.5, 5.0, 6.0 Å, respectively. The presences of hydrogen bonds were determined by strong NOEs in both NOE spectra, as well as their beta-sheet secondary chemical shifts. Each hydrogen bond was constrained with two upper limits of 2.5 and 3.5 Å for HN...O and N...O, respectively. Structural calculations were performed using Xplor-NIH v2.39 (77) from an extended structure with the default anneal.py script. A total of 200 structures were calculated, and the final 20 structures were selected based on the lowest total violation energies.

### Native Mass Spectrometry in Detergent Micelles

tOmpA, OmpAAG, OmpTrans2, and OmpTrans3 proteins were analyzed by native mass spectrometry (MS) using a Thermo Q ExactiveTM Ultrahigh Mass Range (UHMR) Orbitrap® instrument (Thermo Fisher Scientific. Bremen. Germany). Prior to MS analysis, protein samples received in 20 mM Tris, 150 mM NaCl, 0.02% n-Dodecyl-β-D-Maltopyranoside (DDM), pH 8.0 were buffer exchanged into 200 mM ammonium acetate, 2X CMC DDM, pH 8.0 using Micro Bio-Spin® P6 columns with a 6 kDa cutoff (Bio-Rad, Hercules, CA, USA). Proteins were analyzed at concentrations of 3-4 µM monomer. Ions were generated via nano-electrospray ionization using borosilicate capillaries pulled in-house using a micropipette tip puller (Sutter Instruments model P-97, Novato, CA). The protein solution was inserted into the capillary and a platinum wire was inserted into the solution. A spray voltage of 0.5-1.0 kV was used for all experiments. Following ionization, in-source trapping (typically 250-275 V) was used to remove the detergent micelles in the gas phase. Voltages were applied throughout the instrument to optimize ion transmission while minimizing unnecessary ion activation. Mass spectra were collected at a resolution (@: m/z 400) of 12,000 to determine relative ratios of proteins present and at a resolution of 100,000 for confirmation of proteins by accurate mass. Mass spectra were deconvoluted using UniDec version 4.0.0 Beta (78).

### Crystallization

TMB2.17 purified in denaturing conditions was refolded by rapid dilution from 80 µM to 4 µM into a buffer containing 2X CMC of DPC detergent. The solution was incubated at room temperature overnight to allow the proteins to fold and the sample was concentrated to 1 ml using an Amicon Ultra 10 kDa centrifugation device (20 - 25 mg/ml protein). The protein/detergent complex was further purified by SEC on a Superdex 200 increase 10/300 GL column (GE Healthcare) and dialysed against 20 mM Tris 150 mM NaCl pH 8.0, 2X CMC of DPC detergent. Both LCP and classical sitting drops were set up in DPC using Mosquito® LCP by STP Labtech. Diffraction quality crystals appeared in D10 (0.1 M Tris at pH 8.5 and 10 % PEG8000) of MemStart+MemSys® HT by Molecular Dimensions. Crystals were subsequently harvested in a cryo-loop and flash frozen directly in liquid nitrogen for synchrotron data collection.

### Data Collection

Data collection from crystal of TMB2-17 was performed with synchrotron radiation at the Advanced Photon Source (APS), 24ID-E. Crystals belonged to space group R 3 :H with cell dimensions a = b = 51.08 Å, and c = 116.71 Å, α = β = 90° and γ = 120°. X-ray intensities and data reduction were evaluated and integrated using XDS (79) and merged/scaled using Pointless/Aimless in the CCP4 program suite (80).

### Structure Determination and Refinement

Starting phases were obtained by molecular replacement using Phaser® (81) using the designed model. Following molecular replacement, the models were improved using phenix.autobuild (82); efforts were made to reduce model bias by setting rebuild-in-place to false, and using simulated annealing and prime-and-switch phasing. Structures were refined in Phenix®, Model building was performed using COOT (83). The final model was evaluated using MolProbity (84). Structure deposited to PDB (PDB id 6×9Z). Data collection and refinement statistics are recorded in Table 7.

### Supplementary Text

General consideration about the β-barrel architecture

We compared the architecture of the previously designed idealized water-soluble βbarrels with naturally occuring TMBs. We found that these two β-barrel architectures of type (n=8, S=10) share structural similarity that can be associated with the canonical constraints on the β-barrel fold, although they fold into very different environments. Both β-barrel architectures have a common orientation that is defined by the unique structural properties of the β-hairpins on either side of the β-barrel. Because of the chirality of the β-turns, we previously found that the β-strand residues flanking the turns on the bottom side of the water-soluble β-barrels (defined as the side with the N- and C-termini) point towards the surface of the barrel while the β-strand residues flanking the turns on the top of the β-barrel point into the core. Additionally, the β-turns on the two sides of the β-barrels are subject to different constraints on their local twist; the register shifts between each β-hairpin at the bottom of the barrel occur between each β-hairpin and the previous one while at the top they occur between each β-hairpin and the following one. Following these principles that are mostly dictated by the chirality of natural amino acids, the orientation of the TMBs can be easily matched to the orientation of the water-soluble β-barrels.

The bottom side of water-soluble β-barrels structurally match the periplasmic side (cis side) of TMBs: therefore the extracellular (trans) side of TMBs corresponds to the top side of water-soluble β-barrels. We also found similarities in the function of each side of the barrel in both architectures. The bottom side contributes to stability and/or folding. In water-soluble βbarrels, it is often packed with hydrophobic side-chains and features a capping motif with a tryptophan corner critical to folding the protein. The bottom (cis) side of the TMBs feature mostly short β-turns with strongly defined β-turn sequences which might be critical for folding since these interactions form early on in the folding pathway. However, TMBs lack a tryptophan corner folding motif between the first and the last strand by contrast to the water-soluble β-barrel. This difference is discussed later in the supplementary text.

The top side of many water-soluble β-barrels have evolved to support a ligand-binding or catalytic function. To support that function, the core of the β-barrel on the top side is often carved to accommodate the active site and the top β-hairpins are connected with longer loops contributing to the function. TMBs also often feature long and disordered loops on the top (trans) side that support many of the functions attributed to the TMBs. These similarities suggest that structural constraints intrinsic to the β-barrel fold could shape the folding and the stability/function trade-offs in both water-soluble β-barrels and TMBs.

### Rationale for Designing Blueprints and Hydrogen Bonding Constraints for Β-Barrels

The relationship between the number of strands (n) and the shear number (S) of a βbarrel is explained in the main text and illustrated in Table 4. This supplementary material aims to describe a logic to apply to automatically generate blueprints and constraint files for idealized up-and-down β-barrel backbones connected with short β-turns on the cis and trans sides. We previously showed that the β-sheet of β-barrels with the architecture (n=8, S=10) is strained due to the structural constraints of the hydrogen bonds and the tight packing of core residues (5). We described simple rules to design strain-free backbones by introducing glycine kinks at strategic positions in the Cβ-strips and associating each bottom β-turn (or cis β-turn in TMB) with a classic β-bulge at position -2 on the first β-strands; and each top β-turn (or trans β-turn in TMBs) with a G1 β-bulge. As a simple rule-of-thumb to relieve the clashes within the β-strip in the core of the β-barrel, we do not allow more than four side chains in a row in each Cβ-strip. The row of side chains is interrupted by placing a glycine kink (which lacks a side chain) or a register shift (interruption of the hydrogen bond pattern). The four side chain rule originates from two observations: (i) exceptions to this rule are rare in naturally occuring β-barrels of 8 strands and a shear number of 10; (ii) in the β-barrel architecture (n=8, S=10), the vector spanning four residues in the direction of the hydrogen bonds (along the Cβ-strip) and projected to on the plane perpendicular to the main β-barrel axis has a norm of approximately 12.5 Å (equation 4); which represent a quarter of the ideal β-barrel circumference (calculated based on the ideal radius obtained from equation 1). To understand the effect of the number of side chain in a row along a Cβ-strip, it is useful to think about the β-barrel cross-section along the main axis as a 2D geometric shape - where glycine kinks form geometric corners connected by straight lines (which are the rows of side chains in the core Cβ-strips, assuming that the clashes between those side chains favor straight β-sheets). Every additional side chain in a row along a Cβ-strip will increase the length of one side by approximately 3 Å. We reasoned that the a β-barrel cross-section with one long side might be unfavorable because (i) the additional length would have to be accommodated with acute angles which might result in more strain on the glycine kink corners (ii) the increase of the length of one side above 12 Å would result in a decrease of the volume in the core of the β-barrel, which could lead to difficult core pack and to more side chain clashes. It is, however, important to note that the principles above do not apply to other β-barrel architectures.

We further defined ideal β-barrel topologies in the context of membrane-associated architectural constraints. A basic assumption of the provided guidelines is that the entire βbarrel is embedded in the membrane. Hence, the transmembrane span of a β-strand is defined as the number of residues between the cis and trans anchor residues (z). The distance between these two surface residues (z x d; where d is the average distance between two Calphas along a β-strand of 3.3 Å) is projected on the main axis of the β-barrel to calculate the transmembrane span 2 (equation 7, where theta is the angle of the strands to the main axis).

$$Z = z d c o s \left(θ\right) \qquad \text{eq. 7}$$

For a β-barrel of architecture (n=8, S=10), a β-stratid of 11 residues (z=10) will have a transmembrane span Z of approximately 24.1 Å, which is similar to the transmembrane span of TMBs in the outer membrane of E. coli.

Once the length of the transmembrane region of the β-stands has been calculated to match the desired transmembrane span, the total length of each β-strand has to be adjusted to satisfy structural constraints related to the β-barrel architecture. For an ideal β-barrel with as constant as possible distribution of the register shifts, there are several considerations: (i) The previously described principles of ideal β-strand connections (21) state that, for strands connected by short β-turns, the residues flanking the β-turns must form a hydrogen bonded pair. In the context of the β-barrel, this rule implies that the edge residues on cis hairpins point to the surface of the β-barrel (they are the cis anchor residues) while the edge residues on trans hairpins face the core of the β-barrel. Since the transmembrane span of the β-strands is calculated from the cis and trans anchor residues, which are both surface-exposed, the length of each β-strand in the β-barrel is increased by one residue on the trans side.

(ii) To accommodate the β-bulges at the cis side of the β-barrel, the lengths of the β-strands with an odd number must be increased by one residue.

(iii) Because of the up-and-down sequence of β-hairpins and of the tilt of the strands to the βbarrel axis, the odd-numbered strands are shorter than than the even-numbered strands by two residues.

(iv) In the case of a β-barrel architecture (n=8, S=10), the β-strands length has to account for two additional register shifts between cis and trans hairpins as described in the main text. Assuming that the additional register shifts in cis happens after the β-strand N (which must be an odd number), the length of the β-strands N+1, N+2 and N+3 must be increased by two residues.

To summarize, the β-strand lengths of an ideal β-barrel architecture (n=8, S=10) with a βbulge residue associated to every cis β-turn, a transmembrane beta-strand span z and two additional register shifts after the beta-strand N can be calculated as followed:
Length of odd beta-strands: z
Length of even beta-strands: z+2
Length of the odd beta-strand N+2 : z+2
Length of even beta-strands N+1 to N+3: z+4
For example, a β-barrel with a transmembrane span of 24 Å (z=10) and two additional register shifts after strand 1 (N=1) would be described with the following topology: E₁10-L3-E₂14-L2-E₃12-L3-E₄14-L2-E₅10-L3-E₆12-L2-E₇10-L3-E₈12

The constraints describing each backbone hydrogen bond were defined starting from the β-turns. In the absence of a β-turn to guide the strand pairing between the first and the last strand in the β-barrel, the register between these two strands was manually defined to match the desired shear number S. In an ideal β-hairpin connected with a short β-turn (less than six residues long (21)), the last residue on the first β-strand and the first residue on the second βstrand form a hydrogen-bonded pair. One hydrogen bond constraint was designed between the backbone amide of the last residue on the first strand and the backbone carbonyl of the first residue on the second strand (the β-turn flanking residues). For two-residue β-turns (cis side of the β-barrel), a second hydrogen bond was designed between those two residues. For three-residues β-turns (trans side of the β-barrel), the second hydrogen bond was designed between the backbone carbonyl of the last residue on the first strand and the third residue in the β-turn, consistently with the hydrogen bond pattern characteristic of the 3:5 type I β-turn with a G1 β-bulge. Since antiparallel β-strands are characterized by alternating pairs of residues sharing two hydrogen bonds and pairs of residues without hydrogen bonds, two hydrogen bond constraints were designed between every second pair of residues while moving away from the β-turn flanking residues until the end of one of the β-strand. To introduce a β-bulge, an additional hydrogen bond constraint was designed between the backbone amide of the β-bulge residue and the backbone carbonyl of the residue on the neighbor strand forming two regular hydrogen bonds to the residue that follows the β-bulge. The next closest residues forming a hydrogen bonded pair are two positions upstream of the β-bulge residue and two positions downstream of the residue that follows the β-bulge.

### Aromatic Girdle Motifs Placement

The presence of motifs that delimit the cis and trans boundaries of the lipid membrane leaflets has been previously demonstrated (88, 89). We derived a pattern for the cis and trans aromatic girdles, based on observations of naturally occurring TMBs and the analysis of the constructed MSA for homologous β-barrels of 8 β-strands.

On the cis membrane boundary, we found a strong signal for tyrosine at the third position from the end of the strands with even numbers (β-strands in the cis hairpins). The frequency of the tyrosine amino acid is as high as 50% at these positions in the MSA. The second most abundant amino acid is phenylalanine, with only 10% frequency (FIG. 10E). Inspection of crystal structures of naturally occurring TMBs confirmed this trend and showed that these tyrosines specifically adopt a t rotamer so that the phenolic hydroxyl on the tyrosine side-chain points towards the cis water/lipid membrane boundary. To compensate for the four-residue register shift in the β-barrel architecture (n=8,S=10), we placed an additional tyrosine at the second position of the β-turn preceding the large change of register.

Tyrosine was also the most abundant amino acid at the trans membrane anchor positions (last position of the first β-strand in the trans hairpins), although the preference was not as clearly marked (25% tyrosine frequency, FIG. 10F). The tyrosine side-chain again adopts the specific t rotamer in crystal structure to point toward the trans water/lipid membrane boundary. In the crystal structures, the tyrosine often interacts with an asparagine residue located two positions up the neighbor strand. We designed two types of motifs on the trans side of the TMBs alternating between the β-hairpins: i) a tyrosine at the last position of the first β-strand interacting with an asparagine at the third position of the β-turn (G1 bulge position); ii) a tyrosine at the third position from the end of the first β-strand interacting with an asparagine at the first position of the second β-strand; as well as a tryptophan residue at the last position of the first β-strand involved in an aromatic stacking interaction with the tyrosine. The tryptophans were introduced to facilitate biophysical characterization of the designs based on intrinsic fluorescence.

### Glycine and Proline Residues Placement

Previous computational design work on the lipid-exposed surface of tOmpA revealed the key role of surface glycine and prolines in TMBs. However, the exact positions and mechanism by which such residues, which are generally destabilizing to β-strands, can enable TMB folding is unknown. In the main text, we describe the hypothesis made to place surface glycine and proline residues in the designs. The rationale is described in more details in the text below.

### Glycines in the Sheet

The glycines in positions facing the core of the barrel - the glycine kinks - were placed in a strategic way to relieve the strain in the β-sheet and shape the β-barrel lumen as described in a previous paragraph. It is worthwhile to note that the rationale proposed here implies that the number and positions of glycine kinks depend on the strain in the β-sheet and will therefore be different for different β-barrel architectures. The exact relationship between the number and position of glycine kinks, the number of strands in the β-barrel and the shear number requires more investigation.

The high frequency of glycine residues on the surface of TMBs is in striking contrast to water-soluble β-barrels, where solvent-exposed glycines on protein surface are rare. We found a conserved glycine residue on the surface of streptavidin (G74 on PDB structure 1STR), but that position is not solvent-exposed but rather buried amidst a dimerization interface. More examples of surface glycines located at dimerization interfaces are provided by the PDBs 2OVS and SEE2. Excluding glycines involved in non-canonical β-turns or β-bulges, we found only one solvent-exposed glycine on the surface of the PDB 4REV (G175). These very limited data, together with the high contribution of tight aromatic-to-glycine packing interactions in the core to protein stability (“aromatic rescue” (28)), suggest that water-exposed glycines in β-sheet are energetically unfavorable but can be stabilized by hydrophobic interactions. We therefore hypothesized that surface glycines in the β-sheet might be less unfavorable in the hydrophobic environment of the lipid membrane and that the extended torsional space accessible to the glycine amino acid might be able to compensate for the out-of-plane hydrogen bond geometry of glycine kink residues.

### Prolines in the Β-Sheet

Two proline residues were introduced into the TMB designs for different purposes. Pro83 has a similar role to the prolines that were placed in our previous water-soluble β-barrel designs. It was designed in the middle of the longest edge-strand resulting from the 4-residue register shift at the cis side of the β-barrel and aimed to protect the edge strand from non-desired strand-strand associations and re-enforce the designed shear number and topology.

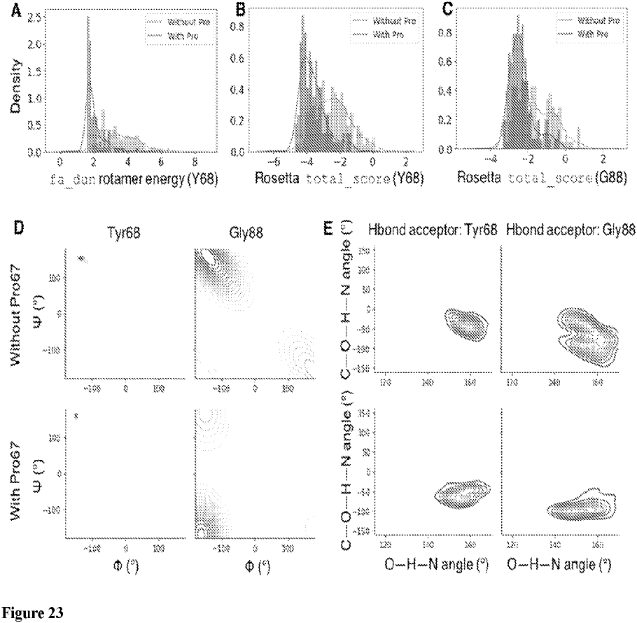

Pro67 was associated to the mortise/tenon motif located in the β-sheet region between the 4-residue cis and trans register shift. We previously observed that, in naturally occurring TMBs, several tyrosines in mortise/tenon motifs are preceded by a proline creating a disruption of the hydrogen bonding pattern in the middle of the β-sheet. We hypothesized that the proline could have a similar role to the surface glycine, relieving the frustration associated with out-of-plane hydrogen bond geometry of the glycine-tyrosine pair and the hydrophobic environment of the lipid membrane. We relaxed TMB design models with and without a proline at position 67 associated with the Tyr68 that forms a mortise/tenon motif with Gly88. We found that in the presence of Pro67, Gly88 adopts a more extended conformation characterized by more negative psi torsion angles and out-of-plane hydrogen bonds (FIGS. 23D,E). To check whether the more extended glycine kink conformation stabilizes the mortise/tenon motif, we analyzed the Rosetta® energy of Tyr68 and Gly88 and found that both residues have in average lower total_score in the models relaxed in the presence of Pro67. Tyr68 had an average lower fa_dun score, indicating that the rotameric state in the motif was stabilized (FIGS. 23A, B).

### Mortise/Renon Folding Motifs

We previously found that the key to the design of water-soluble β-barrels was the strategic placement of specific folding motifs to ensure correct association between β-strands that have ambiguous register definition (such as the interaction between the first and the last β-strands in an up-and-down β-barrel). The tryptophan corner motif was found to tie together the first and last strands of the β-barrel, the longest-range set of interactions and which register is not defined by β-turns. Mutations of the residues belonging to the tryptophan corner into alanine resulted in the failure of the protein to fold into a monomer (5). The tryptophan corner motif is absent from TMBs. The putative folding motifs is the mortise/tenon (29), which was described as a core tyrosine adopting a +60,90 rotamer to closely interact with the grove formed by the glycine kink in an aromatic rescue type of interaction (28) and can be used to predict strand registry (89).

In this work, we used the mortise/tenon in the TMBs designs and made two additional hypotheses regarding the structure and position of the motifs in the protein.

First, we propose to extend the definition of the mortise/tenon motif. The analysis of the generated MSA of homologous sequences to tOmpA showed that the negatively charged residue (aspartate or glutamate) forming a hydrogen bond to the tyrosine is as critical or conserved? as the tyrosine and glycine positions, while the rest of positions involved in the second layer of the polar interaction network are less conserved (FIG. 10C). In naturally occuring TMBs, aromatic residues involved in aromatic rescue interactions appear to be also often stabilized by a cation/pi stacking interaction. However, the cation/pi stacking is an interaction that is poorly captured by Rosetta® energy function and we choose to focus exclusively on the canonical YGD/E motif.

Second, it is unknown which of the ambiguously defined registers in TMBs require a mortise/tenon motif. The topology maps of some naturally occuring TMBs and the positions of the mortise/tenon and comparable motifs are shown in FIGS. 10D,E. We tested two different positions for the mortise/tenon motif in our TMB designs. We propose that the first area of the β-sheet to require a mortise/tenon motif is formed of three β-strands located between the cis and trans four-residue register shifts. In other words, we propose to define ambiguous β-strand registers in TMBs based on uneven distribution of register shifts between hairpins rather than the positions of N- and C-termini as in the water-soluble β-barrels. Indeed, many of the mortise/tenon or comparable motifs were observed in that area in naturally occurring TMBs and the N- and C-terminal interactions do not appear to be critical in TMBs since they can be split and circularly permuted (90, 91), we tested a second mortise/tenon position associated with a glycine kink located closer to the N- and C-termini in our TMB blueprint and on the opposite side of the β-barrel to the first position. The de novo designed TMB sequences have either both or only one of these mortise/tenon motifs.

### Cis and Trans Β-Turns

The design of β-turn sequences is discussed in the main text. Here, we justify the choice of the type of short β-turns (the β-turn backbone conformation and length) used to assemble TMB backbones. These principles are valid for the water-soluble and transmembrane β-barrels, which share similar backbone properties.

We previously showed that β-bulges associated with β-hairpins were necessary to relieve the strain associated with the high curvature of the β-sheet in the β-barrel architecture (n=8, S=10) (5). Since the structural environments of the four β-turns on each side of the β-barrel are similar, the same β-turns and β-bulge positions were used throughout the cis side as well as the trans side. Because of the prefered chirality of ββ connections (21) and the hydrogen bond patterns characteristic to β-bulges (92), the ideal placement of β-bulges is at position -2 from the cis β-turns (preceding the paired β-strand residue at position -1) and position +1 from the trans β-turns (preceding and replacing the β-strand residue at position +1, which now shifts to position +2). We previously found that the type I β-turn (with the ABEGO type sequence AA) is prefered when a β-bulge is located in position -2 (5) and used that type of β-turn to connect cis β-hairpins. The trans β-hairpins were connected with 3:5 type 1 β-turns (with ABEGO type AAG) which feature an intrinsic G1 β-bulge at third position (25), which modifies the hydrogen bonding pattern of the first residue in the second β-strand. This is equivalent to placing a β-bulge at position +1 from the β-turn, and the 3:5 type I β-turn has been both described as a 3-residue turn and a 2-residue turn followed by a β-bulge (92, 93).

### Combinatorial Sequence Design of Set TMB2

The goal of the last set of designs reported here is to increase the hydrophobicity of the core of the TMB designs which will disrupt the alternation of polar and hydrophobic residues along the β-strand and reduce the β-sheet propensity. In short, we started from the mortise/tenon motifs and grew second shell polar interactions to stabilize the tyrosine rotamers. Hydrophobic residues were packed in patches between the resulting polar networks.

To achieve this result, we introduced the tyrosines early in the design process at the first stage of full-atom backbone refinement. Based on our extended definition of the folding motif (YGD/E), we used Rosetta® HBNet (39) to exhaustively search all the positions that can accommodate a negatively charged aspartate or glutamate residue acting as hydrogen bond acceptor to the tyrosines. The YGD/E motifs identified on each backbone were recombined to generate all the possible combinations of one or two motifs per design. We further ran three additional iterations of combinatorial sequence design that aimed to grow second-shell polar networks around the YGD/E motifs. For each iteration, the surface and core of the TMBs were designed independently to limit the time necessary to achieve each step and to be able to quickly re-adjust subsequent design trajectories. All amino acids except cysteine, proline and glycine were allowed for the design of the core with backbone movement enabled (the glycine kinks were introduced at the backbone-building stage). Only hydrophobic amino acids and the aromatic girdle residues were allowed for the surface design stage, with backbone movement and core side-chain repacking enabled. After each core or surface design step, the best designs were selected based on metrics describing the quality of the core networks of polar interactions in terms of their size, energy and robustness.

### Supplementary Information References

66. E. Marcos, B. Basanta, T. M. Chidyausiku, Y. Tang, G. Oberdorfer, G. Liu, G. V. T. Swapna, R. Guan, D.-A. Silva, J. Dou, J. H. Pereira, R. Xiao, B. Sankaran, P. H. Zwart, G. T. Montelione, D. Baker, Principles for designing proteins with cavities formed by curved β sheets. Science. 355, 201-206 (2017).

67. Y.-R. Lin, N. Koga, R. Tatsumi-Koga, G. Liu, A. F. Clouser, G. T. Montelione, D. Baker, Control over overall shape and size in de novo designed proteins. Proc. Natl. Acad. Sci. U. S. A. 112, E5478-85 (2015).

68. H. Park, P. Bradley, P. Greisen, Jr. Y. Liu, V. K. Mulligan, D. E. Kim, D. Baker, F. DiMaio, Simultaneous Optimization of Biomolecular Energy Functions on Features from Small Molecules and Macromolecules. J. Chem. Theory Comput. 12, 6201-6212 (2016).

69. S. Ovchinnikov, H. Kamisetty, D. Baker, Robust and accurate prediction of residue-residue interactions across protein interfaces using evolutionary information. Elife. 3, e02030 (2014).

70. L. Fu, B. Niu, Z. Zhu, S. Wu, W. Li, CD-HIT: accelerated for clustering the next-generation sequencing data. Bioinformatics. 28 (2012), pp. 3150-3152.

71. M. B. Ulmschneider, M. S. P. Sansom, Amino acid distributions in integral membrane protein structures. Biochimica et Biophysica Acta (BBA) - Biomembranes. 1512 (2001), pp. 1-14.

72. D. Gront, D. W. Kulp, R. M. Vernon, C. E. M. Strauss, D. Baker, Generalized fragment picking in Rosetta: design, protocols and applications. PLoS One. 6, e23294 (2011).

73. F. Delaglio, S. Grzesiek, G. W. Vuister, G. Zhu, J. Pfeifer, A. Bax, NMRPipe: a multidimensional spectral processing system based on UNIX pipes. J. Biomol. NMR. 6, 277-293 (1995).

74. Website, (available at Goddard TD, Kneller DG SPARKY 3. University of California, San Francisco. Available at http://www.cgl.ucsf.edu/home/sparky/).

75. S. G. Hyberts, A. G. Milbradt, A. B. Wagner, H. Arthanari, G. Wagner, Application of iterative soft thresholding for fast reconstruction of NMR data non-uniformly sampled with multidimensional Poisson Gap scheduling. J. Biomol. NMR. 52, 315-327 (2012).

76. Y. Shen, A. Bax, Protein backbone and sidechain torsion angles predicted from NMR chemical shifts using artificial neural networks. J. Biomol. NMR 56, 227-241 (2013).

77. C. D. Schwieters, J. J. Kuszewski, G. Marius Clore, Using Xplor-NIH for NMR Molecular Structure Determination. ChemInform. 37 (2006)., doi:10.1002/chin.200644278.

78. M. T. Marty. A. J. Baldwin, E. G. Marklund, G. K. A. Hochberg, J. L. P. Benesch, C. V. Robinson, Bayesian deconvolution of mass and ion mobility spectra: from binary interactions to polydisperse ensembles. Anal. Chem. 87, 4370-4376 (2015).

79. W. Kabsch, XDS. Acta Crystallogr. D Biol. Crystallogr. 66, 125-132 (2010).

80. M. D. Winn, C. C. Ballard, K. D. Cowtan, E. J. Dodson, P. Emsley, P. R. Evans, R. M. Keegan, E. B. Krissinel, A. G. W. Leslie, A. McCoy, S. J. McNicholas, G. N. Murshudov, N. S. Pannu, E. A. Potterton, H. R. Powell, R. J. Read, A. Vagin, K. S. Wilsonc, Overview of the CCP4 suite and current developments. Acta Crystallogr. D Biol. Crystallogr. 67, 235-242 (2011).

81. A. J. McCoy, L. C. Storoni, G. Bunkoczi, R. D. Oeffner, R. J. Read, Phaser crystallographic software. J. Appl. Crystallogr. 40, 658-674 (2007).

82. P. D. Adams, P. V. Afonine, G. Bunkóczi, V. B. Chen, I. W. Davis, N. Echols, J. J. Headd, L-W. Hung, G. J. Kapral, R. W. Grosse-Kunstleve, A. J. McCoy, N. W. Moriarty, R. Oeffner, R. J. Read, D. C. Richardson, J. S. Richardson, T. C. Terwilliger, P. H. Zwart, PHENIX: a comprehensive Python-based system for macromolecular structure solution. Acta Crystallogr. D Biol. Crystallogr. 66, 213-221 (2010).

83. P. Emsley. K. Cowtan, Coot: model-building tools for molecular graphics. Acta Crystallogr. D Biol. Crystallogr. 60, 2126-2132 (2004).

84. C. J. Williams, J. J. Headd, N. W. Moriarty, M. G. Prisant, L. L. Videau, L. N. Deis, V. Verma, D. A. Keedy, B. J. Hintze, V. B. Chen, S. Jain, S. M. Lewis, W. B. Arendall 3rd, J. Snoeyink, P. D. Adams, S. C. Lovell, J. S. Richardson, D. C. Richardson, MolProbity: More and better reference data for improved all-atom structure validation. Protein Sci 27, 293-315, doi:10.1002/pro.3330 (2018).

85. D. R. Flower. The lipocalin protein family: structure and function. Biochemical Journal. 318 (1996), pp. 1-14.

86. L. H. Greene, E. D. Chrysina, L. I. Irons, A. C. Papageorgiou, K. Ravi Acharya, K. Brew, Role of conserved residues in structure and stability: Tryptophans of human serum retinol-binding protein, a model for the lipocalin superfamily. Protein Science. 10 (2009), pp. 2301-2316.

87. J. H. Kleinschmidt, Folding of β-barrel membrane proteins in lipid bilayers - Unassisted and assisted folding and insertion. Biochim. Biophys. Acta. 1848, 1927-1943 (2015).

88. R. Jackups, S. Cheng, J. Liang, Sequence Motifs and Antimotifs in β-Barrel Membrane Proteins from a Genome-Wide Analysis: The Ala-Tyr Dichotomy and Chaperone Binding Motifs. Journal of Molecular Biology. 363 (2006), pp. 611-623.

89. R. Jackups, J. Liang, Interstrand Pairing Patterns in β-Barrel Membrane Proteins: The Positive-outside Rule, Aromatic Rescue, and Strand Registration Prediction. Journal of Molecular Biology. 354 (2005), pp. 979-993.

90. R. Kocbnik, In vivo membrane assembly of split variants of the E.coli outer membrane protein OmpA. The EMBO Journal. 15 (1996), pp. 3529-3537.

91. R. Koebnik, L. Krämer, Membrane Assembly of Circularly Permuted Variants of theE. coliOuter Membrane Protein OmpA. Journal of Molecular Biology. 250 (1995), pp. 617-626.

92. P. Craveur, A. P. Joseph, J. Rebehmed, A. G. de Brevern, β-Bulges: extensive structural analyses of β-sheets irregularities. Protein Sci. 22, 1366-1378 (2013).

93. M. A. Jiménez, Design of monomeric water-soluble β-hairpin and β-sheet peptides. Methods Mol. Biol. 1216, 15-52 (2014).

94. I. Walsh, F. Seno, S. C. E. Tosatto, A. Trovato, PASTA 2.0: an improved server for protein aggregation prediction. Nucleic Acids Res. 42, W301-7 (2014).

95. O. Conchillo-Solé, N. S. de Groot, F. X. Avilés, J. Vendrell, X. Daura, S. Ventura, AGGRESCAN: a server for the prediction and evaluation of “hot spots” of aggregation in polypeptides. BMC Bininformatics. 8, 65 (2007).

96. G. E. Crooks, WebLogo: A Sequence Logo Generator. Genome Research. 14 (2004), pp. 1188-1190.

97. H. Wang, K. K. Andersen, B. S. Vad, D. E. Otzen, OmpA can form folded and unfolded oligomers. Biochim. Biophys. Acta. 1834, 127-136 (2013).

98. R. A. Laskowski, J. Jablonska, L. Pravda, R. S. Vařeková, J. M. Thornton, PDBsum: Structural summaries of PDB entries. Protein Sci. 27, 129-134 (2018).

We claim:

1. A non-naturally occurring beta barrel protein comprising the formula X1-Z1-X2-Z2-X3-Z3-X4-Z4-X5-Z5-X6-Z6-X7-Z7-X8-Z8, wherein:
X1 comprises at least two amino acid residues, wherein the C-terminal residue in X1 is G;
Z1 is a beta strand consisting of 10 amino acid residues, wherein residue 1 is S, T or D, residue 9 is G and residue 10 is W or Y, and wherein residues 2, 4, 6, and 8 are hydrophobic residues or G;
X2 is a loop comprising at least 5 amino acids;
Z2 is a beta strand consisting of 12 amino acid residues, wherein residues 5 and 6 are G, residue 9 is Y, residue 12 is S, T, or D or wherein residue 12 is S or T, and residues 1, 3, 7, and 11 are hydrophobic residues or G;
X3 is a beta turn consisting of two amino acids in length;
Z3 is a beta strand consisting of 9 amino acid residues, wherein residues 6 and 8 are G, residues 7 and 9 are W or Y, and residues 1, 3 and 5 are hydrophobic residues or G;
X4 is a loop comprising at least 5 amino acids;
Z4 is a beta strand consisting of 14 amino acid residues, wherein residue 1 is N or Q, residues 6-8 are G, residue 11 is Y, residue 14 is S, T, or D or wherein residue 14 is S or T, and residues 3, 5, 9, and 13 are hydrophobic residues or G;
X5 is a beta turn consisting of two amino acids in length;
Z5 is a beta strand consisting of 11 amino acid residues, wherein residue 3 is P, residue 8 is G, residue 11 is Y or W, and residues 1, 5, 7, and 9 are hydrophobic residues or G;
X6 is a loop comprising at least 5 amino acids;
Z6 is a beta strand consisting of 14 amino acid residues, wherein residue 3 is P, residues 6 and 8 are G, residue 11 is Y, residue 14 is S, T, or D or wherein residue 14 is S or T, and residues 1, 5, 7, 9, and 13 are hydrophobic residues or G;
X7 is a beta turn consisting of two amino acids in length;
Z7 is a beta strand consisting of 9 amino acid residues, wherein residue 8 is G, residues 7 and 9 is W or Y, and residues 1, 3, and 5 are hydrophobic residues or G;
X8 is a loop comprising at least 5 amino acids;
Z8 is a beta strand consisting of 12 amino acid residues, wherein residue 1 is N or Q, residue 6 is G, residue 9 is Y, and residues 1, 3, 5, 7, and 11 are hydrophobic residues or G.

2. The protein of claim 1, wherein the C-terminal residues in X1 are PG or QG.

3. (canceled)

4. The protein of claim 1, wherein residue 1 in Z1 is S or T.

5. The protein of claim 1, wherein none of X2, X4, X6, or X8 comprise consecutively the amino acid residues across a single row of Table 1.

6. The protein of claim 1, wherein X3, X5, and X7 independently have P, E, or D at residue 1; and N, G, E, D, Q, or Y at position 2.

7. The protein of claim 1, wherein Z1 residue 5 is Y, Z5 residue 4 is Y, or both.

8. The protein of claim 1, wherein X2, X4, X6, or X8 each independently comprise an amino acid sequence selected from the group consisting of the amino acid sequence of SEQ ID NOS:22-26.

9. The protein of claim 1, wherein residue 2 of X2 is Y.

10. The protein of claim 1, wherein one or more of the following is true:
Z1 residue 8 is A;
Z3 residue 5 is A;
Z5 residue 7 is A;
Z6 residue 5 and residue 7 are A or G; and/or
Z8 residue 5 is A or G.

11. The protein of claim 1, wherein one or both of the following is true:
Z3 residue 4 is E or D and Z1 residue 5 is Y; and/or
Z7 residue 6 is E or D and Z5 residue 4 is Y.

12. The protein of claim 1, wherein one or more of X1, X2, X4, X6, and X8 comprise an added functional domain.

13. (canceled)

14. The protein of claim 1, comprising the amino acid sequence at least 50%, 55%, 60%, 65%, 70%, 75%, 80%, 85%, 90%, 95%, or 100% identical to the amino acid sequence selected from SEQ ID NOS:1-19, wherein residues in parentheses are optional and may be present or absent.

15. (canceled)

16. The protein of claim 1, comprising the amino acid sequence at least 50%, 55%, 60%, 65%, 70%, 75%, 80%, 85%, 90%, 95%, or 100% identical to the amino acid sequence selected from SEQ ID NOS:20-21.

17. A protein comprising the amino acid sequence at least 50%, identical to the amino acid sequence selected from SEQ ID NOS:1-21, wherein residues in parentheses are optional and may be present or absent.

18. (canceled)

19. A non-naturally occurring, self-complementing multipartite beta barrel protein, comprising at least a first polypeptide component and a second polypeptide component, wherein the at least first polypeptide component and the second polypeptide component are not covalently linked, wherein in total the at least first polypeptide component and the second polypeptide component comprise domains X1-Z1-X2-Z2-X3-Z3-X4-Z4-X5-Z5-X6-Z6-X7-Z7-X8-Z8, wherein each domain is as defined in claim 1;
wherein (a) each beta strand is fully present within one polypeptide component of the at least first polypeptide component and the second polypeptide component, (b) none of the at least first polypeptide component and the second polypeptide component include each of Z1, Z2, Z3, Z4, Z5, Z6, Z7, and Z8; and (c) one of domains X2, X4, X6, and X8 may be partially or wholly absent in each of the first polypeptide and the second polypeptide.

20. A nucleic acid encoding the beta barrel protein of claim 1.

21. An expression vector comprising the nucleic acid of claim 20 operatively linked to a control sequence.

22. A recombinant host cell comprising the expression vector of claim 21.

23. A pharmaceutical composition, comprising
(a) the beta barrel protein of claim 1; and
(b) a pharmaceutically acceptable carrier.

24. Method for using the beta barrel protein of claim 1 for scaffolding binding epitopes and functional domains on liposomes, cell surface, or detergent micelles, for drug delivery, or as ion, water or small-molecule permeable transmembrane channels.

25. (canceled)


SEQUENCE LISTING (31 sequences)

SEQ ID NO: 1 - protein, 124 aa, Artificial Sequence
  Synthetic
  MISC_FEATURE (1)..(4): optionally absent
  MISC_FEATURE (124)..(124): optionally absent
MQDGPGTLDVFVAAGWNTDNTIEITGGATYQLSPYIMVKAGYGWNNSSLNRFEFGGGLQYKVTPDLEPYAWAGATYNTDNTLVPAAGAGFRYKVSPEVKLVVEYGWNNSSLQFLQAGLSYRIQP

SEQ ID NO: 2 - protein, 124 aa, Artificial Sequence
  Synthetic
  MISC_FEATURE (1)..(4): optionally absent
  MISC_FEATURE (124)..(124): optionally absent
MEQKPGTLMVYVVVGYNTDNTVDVVGGAQYAVSPYLFLDVGYGWNNSSLNFLEVGGGVSYKVSPDLEPYVKAGFEYNTDNTIKPTAGAGALYRVSPNLALMVEYGWNNSSLQKVAIGIAYKVKD

SEQ ID NO: 3 - protein, 124 aa, Artificial Sequence
  Synthetic
  MISC_FEATURE (1)..(3): optionally absent
  MISC_FEATURE (124)..(124): optionally absent
MGQPQGSIAVSVELGYNTDNTISIVGGLSYALSPYLTVRAGYGWNNSSLNELVIGGGIFYQVSPEVEPYIAFGAKFNTDNTLKPFAGAGAAYKVSPELQLVAEYGWNNSSLQEIHVGFEYKLAE

SEQ ID NO: 4 - protein, 124 aa, Artificial Sequence
  Synthetic
  MISC_FEATURE (1)..(4): optionally absent
  MISC_FEATURE (124)..(124): optionally absent
MGTKPGSVWVKVLAGWNTDNTIVFSGGASYALTPYLEIEAGYGWNNSSLNAAFFGGGVMYTVSPDLEPYVWAGAHYNTDNTLKPAAGAGAKYRVTPDFALEARYGWNNSSLQVVEAGVTYKVKD

SEQ ID NO: 5 - protein, 124 aa, Artificial Sequence
  Synthetic
  MISC_FEATURE (1)..(4): optionally absent
  MISC_FEATURE (124)..(124): optionally absent
MPSRPGDLKVYLVAGWNTDNTIKFEGGLRYDVSPYLLLDAGYGWNNSSLNFLKVGGGFAYTLSPDIAPYVLAGATYNTDNTLAPFAGAGFEYRLTPDLAAVIEYGWNNSSLQWLVAGVAYKVKE

SEQ ID NO: 6 - protein, 124 aa, Artificial Sequence
  Synthetic
  MISC_FEATURE (1)..(4): optionally absent
  MISC_FEATURE (124)..(124): optionally absent
MSDKPGSVALTLDIGWNTDNTVDLVGGAVYALSPYLFLEAGYGWNNSSLNVIKFGGGIMYTLSPDLEPYVRVGAKYNTDNTLKPEAGAGFFYKLTPDLKLKIDYGWNNSSLQTAAVGVTYKVQP

SEQ ID NO: 7 - protein, 124 aa, Artificial Sequence
  Synthetic
  MISC_FEATURE (1)..(4): optionally absent
  MISC_FEATURE (124)..(124): optionally absent
MGPKPGSVYLVVEVGYNTDNTFELVGGLMYALSPYLTLSAGYGWNNSSLNTGKVGGGFYYQITPDLEPYVVVGFKFNTDNTVKPSAGAGALYRVSPDVVLRVEYGWNNSSLQVASVGFEYKVKD

SEQ ID NO: 8 - protein, 124 aa, Artificial Sequence
  Synthetic
  MISC_FEATURE (1)..(4): optionally absent
  MISC_FEATURE (124)..(124): optionally absent
MTPKPGSIALLVKVGYNTDNTIRFAGGAMYAVSPYVFVSAGYGWNNSSLNEFEFGGGVSYDLSPELEPYVFAGATYNTDNTIKPFFGAGFFYRVSPEVKGRVEYGWNNSSLQEVAAGLVYKVQG

SEQ ID NO: 9 - protein, 124 aa, Artificial Sequence
  Synthetic
  MISC_FEATURE (1)..(4): optionally absent
  MISC_FEATURE (124)..(124): optionally absent
MGQQPGTVRVFLVAGYNTDNTIVVMGGLQYAVSPYVALEAGYGWNNSSLNFLVIGGGLEYDVSPDIEPYVSLGFMYNTDNTIKPVIGAGAEYRLSPNLAVRIEYGWNNSSLQFVVAGLAYDVQK

SEQ ID NO: 10 - protein, 124 aa, Artificial Sequence
  Synthetic
  MISC_FEATURE (1)..(4): optionally absent
  MISC_FEATURE (124)..(124): optionally absent
MPDKPGSVQLYVKVGYNTDNTLALEGGLDYAVSPYVFLDVGYGWNNSSLNEFVVGGGAKYTLSPELEPYVFAGVKYNTDNTLKPFAGAGAEYRVSPNVKLRIEYGWNNSSLQVLAAGLAYKVRD

SEQ ID NO: 11 - protein, 124 aa, Artificial Sequence
  Synthetic
  MISC_FEATURE (1)..(4): optionally absent
  MISC_FEATURE (124)..(124): optionally absent
MGQKPGSIALFVVAGWNTDNTVELSGGLQYEVSPYVTVDAGYGWNNSSLNFFEAGGGVKYRVTPQLEPYVVAGVRYNTDNTLKPFAGAGAEYKLSPDLALRVEYGWNNSSLQFLRGGLKYQVKD

SEQ ID NO: 12 - protein, 124 aa, Artificial Sequence
  Synthetic
  MISC_FEATURE (1)..(4): optionally absent
  MISC_FEATURE (124)..(124): optionally absent
MPEPPGTVAIVVMVGYNTDNTFDVHGGLSYVLSPYLLVDAGYGWNNSSLNMVHVGGGVQYSGDPDLDPYLTAGVKYNTDNTLKPFAGAGFKYRVTPDLVIRVEYGWNNSSLQEAKVGFEYKLRG

SEQ ID NO: 13 - protein, 124 aa, Artificial Sequence
  Synthetic
  MISC_FEATURE (1)..(4): optionally absent
  MISC_FEATURE (124)..(124): optionally absent
MRPQPGSVSVFLAAGWNTDNTIVIVGGASYKLSPYLELTAGYGWNNSSLNEIEVGGGVEYQLTPEIYPYVFAGAVYNTDNTLRPTAGAGAKYKLSPNLALRADYGWNNSSLQKVKAGVEYTLIP

SEQ ID NO: 14 - protein, 124 aa, Artificial Sequence
  Synthetic
  MISC_FEATURE (1)..(4): optionally absent
  MISC_FEATURE (124)..(124): optionally absent
MGPKPGSLELYVVAGWNTDNTIELKGGLQYAISPYLSLDVGYGWNNSSLNKFEAGGGLEYRLTPEIVPYVKAGLSWNTDNTVKPAAGAGAKYKLSPDLALMIEYGWNNSSLNWLVAGASYKIKD

SEQ ID NO: 15 - protein, 124 aa, Artificial Sequence
  Synthetic
  MISC_FEATURE (1)..(4): optionally absent
  MISC_FEATURE (124)..(124): optionally absent
MQPVPGSVFITVAIGYNTDNTLKIMGGLEYVVSPYGSVVAGYGWNNSSLNEIKVGGGLHYKLSPDIFPYVVAGVVYNTDNTLKPTAGGGVLYKLSPELFARVEYGWNNSSLQEVLVGAAYRVRP

SEQ ID NO: 16 - protein, 124 aa, Artificial Sequence
  Synthetic
  MISC_FEATURE (1)..(4): optionally absent
  MISC_FEATURE (124)..(124): optionally absent
MPKKPGSVEVYVAGGWNTDNTIVIKGGLQYAVSPYFALDVGYGWNNSSLNTGMAGGGFLYVVTPDLEPFVSGGVKFNTDNTAKPMVGAGFTYRLSPNLALRVWYGWNNSSLNEVEAGVSYRVKD

SEQ ID NO: 17 - protein, 124 aa, Artificial Sequence
  Synthetic
  MISC_FEATURE (1)..(4): optionally absent
  MISC_FEATURE (124)..(124): optionally absent
MQDKPGTIRIVVMVGYNTDNTVDVSGGLTYALSPYLKITVGYGWNNSSLNLFEVGGGVEYTISPEVEPYVVAGVKYNTDNTLKPFAGAGFMYRLSPDLAAMVDYGWNNSSLNLARLGFAYKVQD

SEQ ID NO: 18 - protein, 124 aa, Artificial Sequence
  Synthetic
  MISC_FEATURE (1)..(4): optionally absent
  MISC_FEATURE (124)..(124): optionally absent
MQKRPGSVAAFVVAGWNTDNTLHLMGGAEYMLTPYLALRAGYGWNNSSLNTGKAGGGVKYKITPNLEPYIVAGVKWNTDNTVKPFAGAGFDYWLSPNLAITVEYGWNNSSLNEIEAGLSYEVKS

SEQ ID NO: 19 - protein, 124 aa, Artificial Sequence
  Synthetic
  MISC_FEATURE (1)..(4): optionally absent
  MISC_FEATURE (124)..(124): optionally absent
MGTKPGSFALAVAAGWNTDNTIVLVGGIRYSLSPYLFIEAGYGWNNSSLNFLFAGGGVSYQLSPDLEPYAAAGFLYNTDNTIAPWAGAGAKYRLTPDLEADVFYGWNNSSLQFIVAGLEYDVKP

SEQ ID NO: 20 - protein, 173 aa, Artificial Sequence
  Synthetic
  MISC_FEATURE (1)..(1): optionally absent
MQDGPGTLDVFVAAGWNQYHDTGFINNNGPTHENKIEITGGATYQLSPYIMVKAGYGWDGRMPYKGSVENGAYKRNRFEFGGGLQYKVTPDLEPYAWAGATYERADTKSNVYGKNHDNKLVPAAGAGFRYKVSPEVKLVVEYGWKNNIGDAHTIGTRPDKQFLQAGLSYRIQP

SEQ ID NO: 21 - protein, 173 aa, Artificial Sequence
  Synthetic
  MISC_FEATURE (1)..(1): optionally absent
MEQKPGTLMVYVVVGYEQYHDTGFINNNGPTHENKVDVVGGAQYAVSPYLFLDVGYGWTGRMPYKGSVENGAYKKNFLEVGGGVSYKVSPDLEPYVKAGFEYERADTKSNVYGKNHDNRIKPTAGAGALYRVSPNLALMVEYGWKNNIGDAHTIGTRPDKQKVAIGIAYKVKD

SEQ ID NO: 22 - protein, 5 aa, Artificial Sequence
  Synthetic
NTDNT

SEQ ID NO: 23 - protein, 5 aa, Artificial Sequence
  Synthetic
NNSSL

SEQ ID NO: 24 - protein, 5 aa, Artificial Sequence
  Synthetic
TGQSG

SEQ ID NO: 25 - protein, 5 aa, Artificial Sequence
  Synthetic
DSWNK

SEQ ID NO: 26 - protein, 9 aa, Artificial Sequence
  Synthetic
ARDNWNYIP

SEQ ID NO: 27 - protein, 21 aa, Artificial Sequence
  Synthetic
QPRSPYTTVKTDDTTYDSPTQ

SEQ ID NO: 28 - protein, 21 aa, Artificial Sequence
  Synthetic
YQVNPYVYPITDDLYAITPEI

SEQ ID NO: 29 - protein, 12 aa, Artificial Sequence
  Synthetic
YSPYYSPDYSPD

SEQ ID NO: 30 - protein, 144 aa, Artificial Sequence
  Synthetic
MQDGPGTLDVFVAAGWNTDNTIEITGGATYQLSPYIMVKAGYGWNNSSLNRFEFGGGLQYKVTPDLEPYAWAGATYNTDNTLVPAAGAGFRYKVSPEVKLVVEYGWNNSSLQFLQAGLSYRIQPMQDGPGTLDVFVAAGWNTDN

SEQ ID NO: 31 - protein, 102 aa, Artificial Sequence
  Synthetic
APKDNTWYTGAKLGWLGAGAFGGYQVNPYVGFEMGYDWQGVQLTAKLGYPITDDLDIYTRLGGMVVSPVFAGGVEYAITPEIATRLEYQWGMLSLGVSYRFG